(19) United States
(12) Patent Application Publication
(10) Pub. No.: US 2016/0108406 A1
(43) Pub. Date: Apr. 21, 2016

(54) METHOD OF REGULATING CFTR EXPRESSION AND PROCESSING

(71) Applicant: UNIVERSITY OF IOWA RESEARCH FOUNDATION, Iowa City, IA (US)

(72) Inventors: Paul B. McCray, Iowa City, IA (US); Shyam Ramachandran, Iowa City, IA (US)

(73) Assignee: UNIVERSITY OF IOWA RESEARCH FOUNDATION, Iowa City, IA (US)

(21) Appl. No.: 14/878,829

(22) Filed: Oct. 8, 2015

Related U.S. Application Data
(60) Provisional application No. 62/061,500, filed on Oct. 8, 2014.

(51) Int. Cl. C12N 15/113; A61K 31/7088; A61K 45/06; A61M 15/00

(52) U.S. Cl. CPC C12N 15/1137; A61M 15/00; C12N 15/113; A61K 31/7088; A61K 45/06; A61M 2202/064; C12N 2310/14; C12N 2310/321

(57) ABSTRACT

The present invention relates to methods of reducing ΔF508-CFTR ubiquitination or degradation, or increasing ΔF508-CFTR processing or function in a CF cell comprising contacting the cell with a therapeutic agent that inhibits NEDD8, FBXO2, and/or SYVN1 expression in the cell.

### RELATED APPLICATIONS

This application claims the benefit of priority of U.S. Provisional Application Ser. No. 62/061,500 filed on Oct. 8, 2014, which application is herein incorporated by reference.

### STATEMENT REGARDING FEDERALLY SPONSORED RESEARCH

This invention was made with government support under grant R21 HL104337 awarded by the National Institutes of Health. The government has certain rights in the invention.

### BACKGROUND OF THE INVENTION

Cystic fibrosis (also known as CF or mucoviscidosis) is a common recessive genetic disease which affects the entire body, causing progressive disability and often early death. The name cystic fibrosis refers to the characteristic scarring (fibrosis) and cyst formation within the pancreas, first recognized in the 1930s. Difficulty breathing is the most serious symptom and results from frequent lung infections that are treated with, though not cured by, antibiotics and other medications. A multitude of other symptoms, including sinus infections, poor growth, diarrhea, and infertility result from the effects of CF on other parts of the body.

CF is caused by a mutation in the gene that encodes the cystic fibrosis transmembrane conductance regulator (CFTR) protein. This gene is required to regulate the components of sweat, digestive juices, and mucus. The CFTR protein, when positioned properly in the cell membrane, opens channels in the cell membrane. When the channels open, anions, including chloride and bicarbonate are released from the cells. Water follows by means of osmosis. Although most people without CF have two functional copies (alleles) of the CFTR gene, only one is needed to prevent cystic fibrosis (i.e., CF is an autosomal recessive disease). CF develops when neither allele can produce a functional CFTR protein. The most common mutation, ΔF508, is a deletion (Δ) of three nucleotides that results in a loss of the amino acid phenylalanine (F) at the 508th (508) position on the protein. The ΔF508 mutation can prevent the CFTR from moving into its proper position in the cell membrane. This mutation causes an abnormal biogenesis and premature degradation of CFTR protein by the cells quality control system and, as a result, there is a paucity/absence of CFTR in the apical membrane of CF epithelial cells. This results in decreased anion permeability across CF epithelia.

CF is most common among Caucasians; one in 25 people of European descent carry one allele for CF. Approximately 30,000 Americans have CF, making it one of the most common life-shortening inherited diseases in the United States. Individuals with cystic fibrosis can be diagnosed before birth by genetic testing or by a sweat test in early childhood. Ultimately, lung transplantation is often necessary as CF worsens. The ΔF508 mutation accounts for two-thirds (66-70%) of CF cases worldwide and 90 percent of cases in the United States; however, there are over 1,500 other mutations that can produce CF.

Currently, there are no cures for cystic fibrosis, although there are several treatment methods. The management of cystic fibrosis has improved significantly over the years. While infants born with cystic fibrosis 70 years ago would have been unlikely to live beyond their first year, infants today are likely to live well into adulthood. The cornerstones of management are proactive treatment of airway infection and inflammation, and encouragement of good nutrition and an active lifestyle. Management of cystic fibrosis is aimed at maximizing organ function, and therefore quality of life. At best, current treatments delay the decline in organ function. Targets for therapy are the lungs, gastrointestinal tract (including pancreatic enzyme supplements), the reproductive organs (including assisted reproductive technology (ART)) and psychological support.

The most consistent aspect of therapy in cystic fibrosis is limiting and treating the lung damage caused by thick mucus and infection, with the goal of maintaining quality of life. Intravenous, inhaled, and oral antibiotics are used to treat chronic and acute infections. Mechanical devices and inhalation medications are used to alter and clear the thickened mucus. These therapies, while effective, can be extremely time-consuming for the patient. One of the most important battles that CF patients face is finding the time to comply with prescribed treatments while balancing a normal life.

In addition, therapies such as transplantation and gene therapy aim to cure some of the effects of cystic fibrosis. Gene therapy aims to introduce normal CFTR to airway epithelial cells. There are two types of CFTR gene therapies under development, the first uses viral vectors (adenovirus, adeno-associated virus or retrovirus) and the second uses plasmid DNA in formulations such as liposomes. However there are problems associated with both of these methods involving efficiency (liposomes insufficient plasmid DNA) and delivery (virus vectors provoke an immune responses).

Accordingly, a more effective, simple-to-administer, and efficient treatment for CF is needed.

### SUMMARY OF THE INVENTION

The present invention provides in certain embodiments, a method of reducing ΔF508-CFTR ubiquitination or degradation, or increasing ΔF508-CFTR processing or function in a CF cell comprising contacting the cell with a NEDD8 therapeutic agent that inhibits NEDD8 expression in the cell. In certain embodiments, the agent comprises an anti-NEDD8 RNAi molecule, an anti-NEDD8 antisense oligonucleotide (ASO), or other agent that suppresses NEDD8 expression, which methods are well-known to those with skill in the art. In yet another embodiment, the method comprises contacting the cell with a NEDD8 therapeutic agent, wherein the agent comprises a small molecule drug that interferes with NEDD8 activity or whose actions mimics the biological effects of NEDD8 suppression. In certain embodiments, NEDD8 expression is inhibited by at least about 10%, 20%, 30%, 40%, 50%, 60%, 70%, 80%, 90% 95%, or 99%. In certain embodiments, small molecule drugs that inhibit NEDD8 activity are used to inhibit NEDD8, such as by inhibiting translation of NEDD8 or by directly interfering with function of the NEDD8 protein. In certain embodiments, the present invention further provides contacting the cell with a FBXO2 therapeutic agent that inhibits FBXO2 expression in the cell. In certain embodiments, the agent comprises an anti-FBXO2 RNAi molecule, an anti-FBXO2 antisense oligonucleotide (ASO), or other agent that suppresses FBXO2 expression. In yet another embodiment, the method comprises contacting the cell with a FBXO2 therapeutic agent, wherein the agent comprises a small molecule drug that interferes with FBXO2 activity or whose actions mimics the biological effects of FBXO2 suppression. In certain embodiments, FBXO2 expression is inhibited by at least about 10%, 20%, 30%, 40%, 50%, 60%, 70%, 80%, 90% 95%, or 99%. In certain embodiments, small molecule drugs that inhibit FBXO2 activity are used to inhibit FBXO2, such as by inhibiting translation of FBXO2 or by directly interfering with function of the FBXO2 protein. In certain embodiments, the present invention further provides contacting the cell with a therapeutic agent that inhibits SYVN1 expression in the cell. In certain embodiments, the agent comprises an anti-SYVN1 RNAi molecule, an anti-SYVN1 antisense oligonucleotide (ASO), or other agent that suppresses SYVN1 expression. In yet another embodiment, the method comprises contacting the cell with a SYVN1 therapeutic agent, wherein the agent comprises a small molecule drug that interferes with SYVN1 activity or whose actions mimics the biological effects of SYVN1 suppression. In certain embodiments, SYVN1 expression is inhibited by at least about 10%, 20%, 30%, 40%, 50%, 60%, 70%, 80%, 90% 95%, or 99%. In certain embodiments, small molecule drugs that inhibit SYVN1 activity are used to inhibit SYVN1, such as by inhibiting translation of SYVN1 or by directly interfering with function of the SYVN1 protein.

The present invention provides in certain embodiments, a method of reducing ΔF508-CFTR ubiquitination or degradation, or increasing ΔF508-CFTR processing or function in a CF cell comprising contacting the cell with a FBXO2 therapeutic agent that inhibits FBXO2 expression in the cell. In certain embodiments, the agent comprises an anti-FBXO2 RNAi molecule, an anti-FBXO2 antisense oligonucleotide (ASO), or other agent that suppresses FBXO2 expression. In yet another embodiment, the method comprises contacting the cell with a FBXO2 therapeutic agent, wherein the agent comprises a small molecule drug that interferes with FBXO2 activity or whose actions mimics the biological effects of FBXO2 suppression. In certain embodiments, FBXO2 expression is inhibited by at least about 10%, 20%, 30%, 40%, 50%, 60%, 70%, 80%, 90% 95%, or 99%. In certain embodiments, small molecule drugs that inhibit FBXO2 activity are used to inhibit FBXO2, such as by inhibiting translation of FBXO2 or by directly interfering with function of the FBXO2 protein. In certain embodiments, the present invention further provides contacting the cell with a therapeutic agent that inhibits SYVN1 expression in the cell. In certain embodiments, the agent comprises an anti-SYVN1 RNAi molecule, an anti-SYVN1 antisense oligonucleotide (ASO), or other agent that suppresses SYVN1 expression. In yet another embodiment, the method comprises contacting the cell with a SYVN1 therapeutic agent, wherein the agent comprises a small molecule drug that interferes with SYVN1 activity or whose actions mimics the biological effects of SYVN1 suppression. In certain embodiments, SYVN1 expression is inhibited by at least about 10%, 20%, 30%, 40%, 50%, 60%, 70%, 80%, 90% 95%, or 99%. In certain embodiments, small molecule drugs that inhibit SYVN1 activity are used to inhibit SYVN1, such as by inhibiting translation of SYVN1 or by directly interfering with function of the SYVN1 protein.

The present invention provides in certain embodiments, a method of reducing ΔF508-CFTR ubiquitination or increasing ΔF508-CFTR processing and function in a CF cell comprising contacting the cell with a SYVN1 therapeutic agent that inhibits SYVN1 and an AHSA1 therapeutic agent that inhibits AHSA1 expression in the cell. In certain embodiments, the agent comprises an anti-SYVN1 RNAi molecule, an anti-SYVN1 antisense oligonucleotide (ASO), or other agent that suppresses SYVN1 expression. In yet another embodiment, the method comprises contacting the cell with a SYVN1 therapeutic agent, wherein the agent comprises a small molecule drug that interferes with SYVN1 activity or whose actions mimics the biological effects of SYVN1 suppression. In certain embodiments, SYVN1 expression is inhibited by at least about 10%, 20%, 30%, 40%, 50%, 60%, 70%, 80%, 90% 95%, or 99%. In certain embodiments, small molecule drugs that inhibit SYVN1 activity are used to inhibit SYVN1, such as by inhibiting translation of SYVN1 or by directly interfering with function of the SYVN1 protein. In certain embodiments, the agent comprises an anti-AHSA1 RNAi molecule, an anti-AHSA1 antisense oligonucleotide (ASO), or other agent that suppresses AHSA1 expression. In yet another embodiment, the method comprises contacting the cell with a AHSA1 therapeutic agent, wherein the agent comprises a small molecule drug that interferes with AHSA1 activity or whose actions mimics the biological effects of AHSA1 suppression. In certain embodiments, AHSA1 expression is inhibited by at least about 10%, 20%, 30%, 40%, 50%, 60%, 70%, 80%, 90% 95%, or 99%. In certain embodiments, small molecule drugs that inhibit AHSA1 activity are used to inhibit AHSA1, such as by inhibiting translation of AHSA1 or by directly interfering with function of the AHSA1 protein.

The present invention provides in certain embodiments, a method of reducing ΔF508-CFTR ubiquitination or degradation, or increasing membrane stability of ΔF508-CFTR in a Cystic Fibrosis (CF) cell comprising contacting the cell with (a) a therapeutic agent that inhibits SYVN1 expression in the cell and (b) a CFTR corrector and/or CFTR potentiator.

SYVN1 is also called Hrd1 or E3 ubiquitin ligase; FBXO2 is also called Fbs1 or E3 ubiquitin ligase); and the interaction between NEDD8 and other proteins.

In certain embodiments, the cell is a CF epithelial cell, such as an airway epithelial cell (e.g., a lung cell, a nasal cell, a tracheal cell, a bronchial cell, a bronchiolar or alveolar epithelial cell). In certain embodiments, the airway epithelial cells are present in a mammal. In certain embodiments, the cell produces a CFTR protein with a phenylalanine deletion at position 508.

In certain embodiments the present invention provides a method of treating a subject having CF comprising administering to the subject an effective amount of a therapeutic agent to alleviate the symptoms of CF, wherein the agent comprises an anti-NEDD8 RNAi molecule, and/or an anti-NEDD8 antisense oligonucleotide (ASO) or other agent that suppresses NEDD8 expression, a small molecule drug that interferes with NEDD8 activity or whose actions mimic the biological effects of NEDD8 suppression; an anti-FBXO2 RNAi molecule, and/or an anti-FBXO2 antisense oligonucleotide (ASO) or other agent that suppresses FBXO2 expression, a small molecule drug that interferes with FBXO2 activity or whose actions mimic the biological effects of FBXO2 suppression; and/or an anti-SYVN1 RNAi molecule, and/or an anti-SYVN1 antisense oligonucleotide (ASO) or other agent that suppresses SYVN1 expression, a small molecule drug that interferes with SYVN1 activity or whose actions mimic the biological effects of SYVN1 suppression.

In certain embodiments, the present invention provides a method for increasing chloride ion conductance in airway epithelial cells of a subject afflicted with cystic fibrosis, wherein the subject's CFTR protein has a loss of phenylalanine at position 508, the method comprising administering to the subject a therapeutic agent, wherein the agent comprises an anti-NEDD8 RNAi molecule, and/or an anti-NEDD8 antisense oligonucleotide (ASO) or other agent that suppresses NEDD8 expression, a small molecule drug that interferes with NEDD8 activity or whose actions mimic the biological effects of NEDD8 suppression; an anti-FBXO2 RNAi molecule, and/or an anti-FBXO2 antisense oligonucleotide (ASO) or other agent that suppresses FBXO2 expression, a small molecule drug that interferes with FBXO2 activity or whose actions mimic the biological effects of FBXO2 suppression; and/or an anti-SYVN1 RNAi molecule, and/or an anti-SYVN1 antisense oligonucleotide (ASO) or other agent that suppresses SYVN1 expression, a small molecule drug that interferes with SYVN1 activity or whose actions mimic the biological effects of SYVN1 suppression. In certain embodiments, the composition further comprises a standard cystic fibrosis pharmaceutical, such as an antibiotic.

In certain embodiments, the agent is administered orally or by inhalation. In certain embodiments, the administration is via aerosol, dry powder, bronchoscopic instillation, intra-airway (tracheal or bronchial) aerosol or orally. In certain embodiments, the epithelial cells are intestinal cells, and may be present in a mammal. In certain embodiments, the agent is administered orally.

In certain embodiments, the present invention provides a therapeutic agent comprising an anti-NEDD8 RNAi molecule, and/or an anti-NEDD8 antisense oligonucleotide (ASO) or other agent that suppresses NEDD8 expression, a small molecule drug that interferes with NEDD8 activity or whose actions mimic the biological effects of NEDD8 suppression for use in treating CF and restoring function to the ΔF508 protein. As used herein the term “restoring function” means that at least 5%-100% of the protein is active. Restored function indicates that the misfolded mutant ΔF508 protein has been rescued from degradation in the proteosome, and successfully trafficked to the cell membrane where it forms a partially functional anion channel. Here it is able to conduct anions such as chloride and bicarbonate.

In certain embodiments, the present invention provides a therapeutic agent comprising an anti-FBXO2 RNAi molecule, and/or an anti-FBXO2 antisense oligonucleotide (ASO) or other agent that suppresses FBXO2 expression, a small molecule drug that interferes with FBXO2 activity or whose actions mimic the biological effects of FBXO2 suppression for use in treating CF and restoring function to the ΔF508 protein. As used herein the term “restoring function” means that at least 5%400% of the protein is active. Restored function indicates that the misfolded mutant ΔF508 protein has been rescued from degradation in the proteosome, and successfully trafficked to the cell membrane where it forms a partially functional anion channel. Here it is able to conduct anions such as chloride and bicarbonate.

In certain embodiments, the invention provides a pharmaceutical composition for treatment of cystic fibrosis, comprising an anti-NEDD8 RNAi molecule, and/or an anti-NEDD8 antisense oligonucleotide (ASO) or other agent that suppresses NEDD8 expression, a small molecule drug that interferes with NEDD8 activity or whose actions mimic the biological effects of NEDD8 suppression in combination with a pharmaceutically acceptable carrier. In certain embodiments the pharmaceutical composition further comprises (a) an anti-FBXO2 RNAi molecule, and/or an anti-FBXO2 antisense oligonucleotide (ASO) or other agent that suppresses FBXO2 expression, a small molecule drug that interferes with FBXO2 activity or whose actions mimic the biological effects of FBXO2 suppression, and/or (b) an anti-SYVN1 RNAi molecule, and/or an anti-SYVN1 antisense oligonucleotide (ASO) or other agent that suppresses SYVN1 expression, or a small molecule drug that interferes with SYVN1 activity or whose actions mimic the biological effects of SYVN1 suppression.

In certain embodiments, the present invention provides a use of a therapeutic agent comprising an anti-NEDD8 RNAi molecule, and/or an anti-NEDD8 antisense oligonucleotide (ASO) or other agent that suppresses NEDD8 expression, a small molecule drug that interferes with NEDD8 activity or whose actions mimic the biological effects of NEDD8 suppression in combination with a pharmaceutically acceptable carrier to prepare a medicament useful for treating CF in an animal. In certain embodiments the present invention further provides they use of (a) an anti-FBXO2 RNAi molecule, and/or an anti-FBXO2 antisense oligonucleotide (ASO) or other agent that suppresses FBXO2 expression, a small molecule drug that interferes with FBXO2 activity or whose actions mimic the biological effects of FBXO2 suppression, and/or (b) an anti-SYVN1 RNAi molecule, and/or an anti-SYVN1 antisense oligonucleotide (ASO) or other agent that suppresses SYVN1 expression, a small molecule drug that interferes with SYVN1 activity or whose actions mimic the biological effects of SYVN1 suppression to prepare a medicament useful for treating CF in an animal.

The present invention further provides a method of substantially restoring CFTR anion channel function in order to provide a therapeutic effect. As used herein the term “substantially restoring” or “substantially restored” refers to increasing the expression of the target gene or target allele by at least about 5%, 10%, 15%, 20%, 25%, 30%, 35%, 40%, 45%, 50%, 55%, 60%, 65%, 70%, 75%, 80%, 85% to 100%. As used herein “increased expression” means that the amount of mRNA is increased, the amount of protein is increased and/or the activity of the protein is increased as compared to CFTRΔF508. As used herein the term “therapeutic effect” refers to a change in the associated abnormalities of the disease state, including pathological and behavioral deficits; a change in the time to progression of the disease state; a reduction, lessening, or alteration of a symptom of the disease; or an improvement in the quality of life of the person afflicted with the disease. Therapeutic effects can be measured quantitatively by a physician or qualitatively by a patient afflicted with the disease state targeted by the therapeutic agent.

### BRIEF DESCRIPTION OF DRAWINGS

FIGS. 1A-1F. SYVN1 and NEDD8 knockdown restore partial ΔF508-CFTR function in CFBE cells. (A, D) Surface display of ΔF508-CFTR in HeLa cells measured by cell-surface ELISA 24 hr after indicated treatments. Fold increase and significance relative to siScr (scrambled) transfection. C18 (6 μM) administered for 24 hr. n=48 (B, E) Representative immunoblot depicting ΔF508-CFTR expression in CFBE cells. C=band C, B=band B, t=α-tubulin. Protein harvested 72 hr post-treatment. Densitometry represents fold increase of ΔF508-CFTR bands C and B in CFBE cells relative to siScr. n=4. C18 (6 μM) administered for 24 hr. (C, F) Change in transepithelial current (Iₜ) in response to Forskolin & IBMX (F&I) treatment in polarized ALI cultures of CFBE cells. Minimum n=6, or mentioned. C18 (6 μM) administered basolaterally 24 hr prior to electrophysiology study. All panels: error bars indicate standard error; statistical significance determined by the Holm-Bonferroni method; *P<0.05.

FIGS. 2A-2E. SYVN1/NEDD8 knockdown restore ΔF508-CFTR function cooperatively with C18/27° C. (A) Surface display of ΔF508-CFTR in HeLa cells measured by cell-surface ELISA 72 hr after indicated treatments. Fold increase and significance relative to siScr (scrambled) transfection. C18 (6 μM) administered for 24 hr; low temperature (27° C.) administered for 24 hr. n=18 (B) Membrane stability of ΔF508-CFTR in HeLa cells measured by pulse-chase cell-surface ELISA 72 hr after indicated treatments. Chase performed at 37° C. n=18 (C) CFTR-ΔF508 ubiquitination measured 72 hr after indicated treatments. CFTR immunoprecipitated with anti-HA antibody and ubiquitin measured with anti-ubiquitin antibody. C18 (6 μM) and 27° C. administered 24 hr prior to harvesting protein. Densitometry relative to siScr. n=4. (D) Representative immunoblot depicting ΔF508-CFTR expression in CFBE cells. C=band C, B=band B, t=α-tubulin. Protein harvested 72 hr post-treatment. Densitometry representing fold increase of ΔF508-CFTR bands C and B relative to siScr in CFBE cells. n=4. (E) Change in current (Iₜ) in response to F&I treatment in polarized ALI cultures of CFBE cells. N=indicated. C18 (6 μM) and 27° C. treatment for 24 hr prior to electrophysiology study. All panels: error bars indicate standard error; statistical significance determined by the Holm-Bonferroni method; *P<0.05, ^{#}P<0.05 (relative to 27° C.), ^{@}P<0.05 (relative to siSYVN1), ^{$}P<0.05 (relative to siNEDD8), ^{&}P<0.05 (relative to C18).

FIGS. 3A-3F. SYVN1 restores ΔF508-CFTR biosynthesis in part via the RNF5/AMFR pathway. (A, E) Surface display of ΔF508-CFTR in HeLa cells measured by cell-surface ELISA 72 hr after indicated treatments. Fold increase and significance relative to siScr transfection. n=18 (B) Representative immunoblot depicting ΔF508-CFTR expression in CFBE cells. C=band C, B=band B, t=α-tubulin. Protein harvested 72 hr post-treatment. Densitometry representing fold increase of ΔF508-CFTR bands C and B relative to siScr in CFBE cells. n=4. (C) Change in transepithelial current (Iₜ) in response to F&I treatment in polarized ALI cultures of CFBE cells. n=6. (D, F) CFTR-ΔF508 ubiquitination measured 72 hr after indicated treatments. CFTR immunoprecipitated with anti-HA antibody and ubiquitin measured with anti-ubiquitin antibody. Densitometry relative to siScr. n=4. All panels: error bars indicate standard error; statistical significance determined by the Holm-Bonferroni method; *P<0.05.

FIGS. 4A-4D. NEDD8 and FBXO2 exhibit overlapping action in rescuing ΔF508-CFTR biosynthesis. (A) Surface display of ΔF508-CFTR in HeLa cells measured by cell-surface ELISA 72 hr after indicated treatments. Fold increase and significance relative to siScr transfection. n=18 (B) Representative immunoblot depicting ΔF508-CFTR expression in CFBE cells. C=band C, B=band B, t=α-tubulin. Protein harvested 72 hr post-treatment. Densitometry representing fold increase of ΔF508-CFTR bands C and B relative to siScr in CFBE cells. n=4. (C) Change in current (Iₜ) in response to F&I treatment in polarized ALI cultures of CFBE cells. n=6. (D) ΔF508-CFTR ubiquitination measured 72 hr after indicated treatments. CFTR immunoprecipitated with anti-HA antibody and ubiquitin measured with anti-ubiquitin antibody. Densitometry relative to siScr. n=4. All panels: error bars indicate standard error; statistical significance determined by the Holm-Bonferroni method; *P<0.05.

FIGS. 5A-5B. Inhibition of SYVN1, NEDD8 or FBXO2 partially restore ΔF508-CFTR function in primary airway epithelial cell cultures. (A) Change in current (Iₜ) in response to F&I treatment in polarized primary airway epithelial cell cultures. Minimum n=4, donors per treatment indicated. Error bars indicate standard error; statistical significance determined by the Holm-Bonferroni method; *P<0.05. (B) Schematic proposing a mechanism by which SYVN1, NEDD8, or FBXO2 inhibition might restore ΔF508-CFTR biosynthesis.

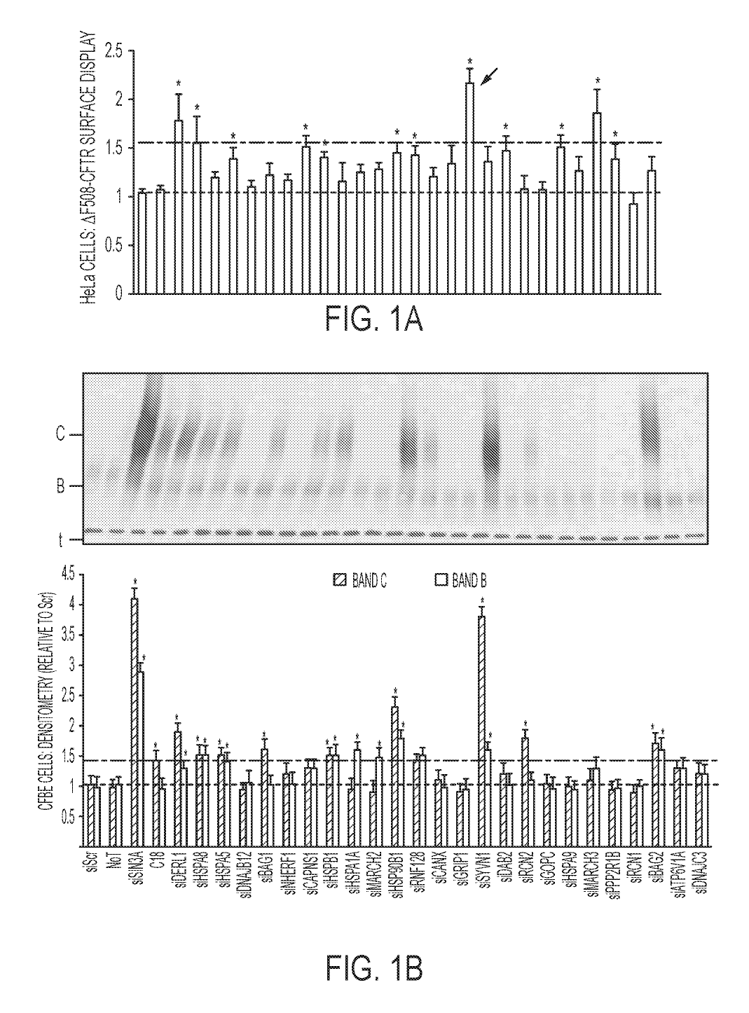

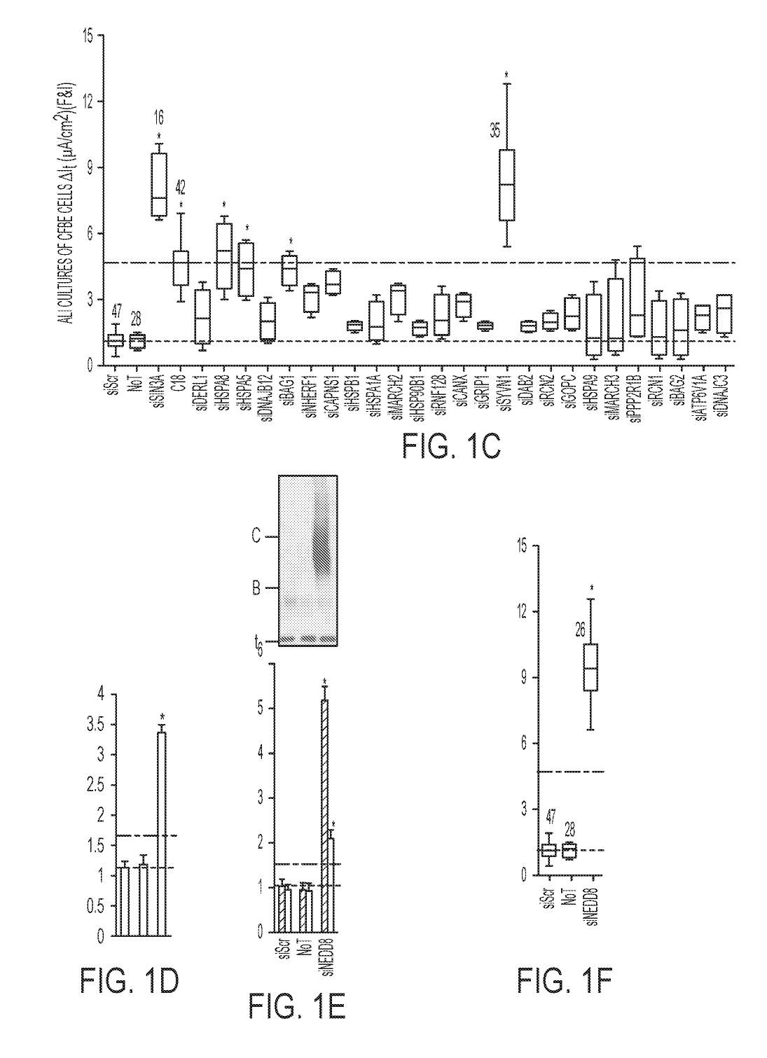

Supplementary FIG. 1. Selection of 25 candidate genes for RNA interference based screening. SIN3A inhibition and miR-138 overexpression in Calu-3 cells resulted in 2809 and 2840 differentially expressed genes respectively. On intersecting with the CFTR associated gene network (a list of 362 hand curated genes) 125 genes exhibited significant enrichment. Of these 25 genes were selected for further RNA interference based studies.

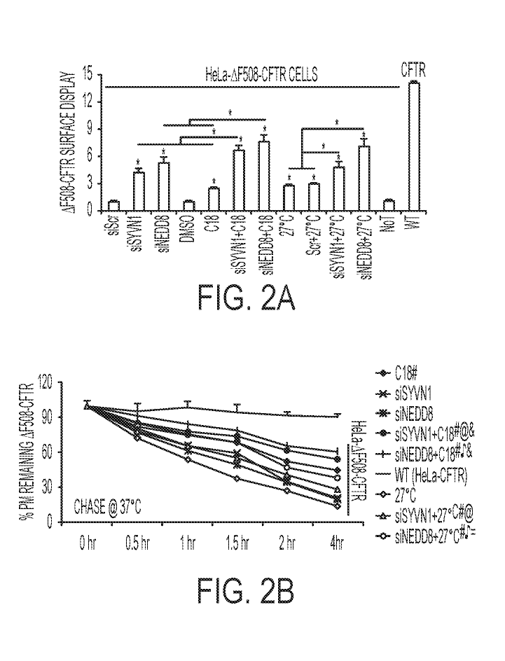

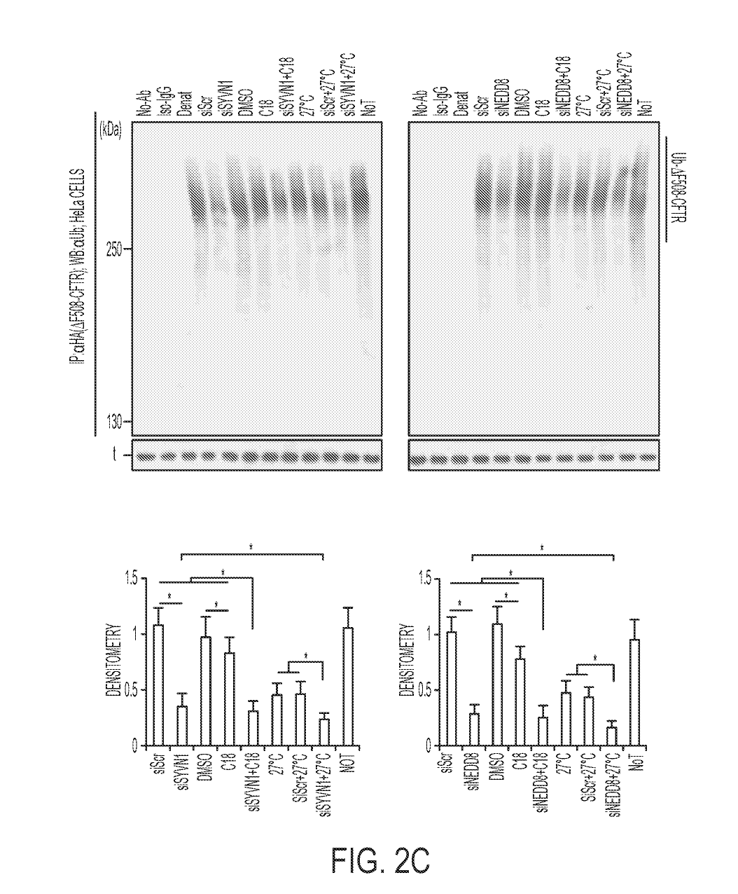

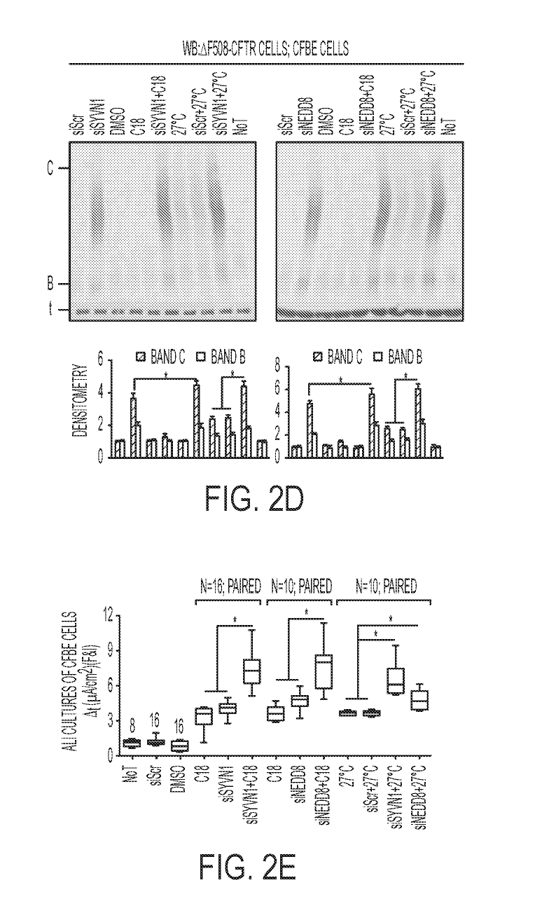

Supplementary FIG. 2. Two DsiRNAs were selected per gene. Remaining mRNA levels of noted genes, relative to the scrambled (siScr) control in CFBE cells, measured by RT-qPCR 24 hr post-transfection. N=4. Error bars indicate standard error; statistical significance determined by the Holm-Bonferroni method; *P<0.05.

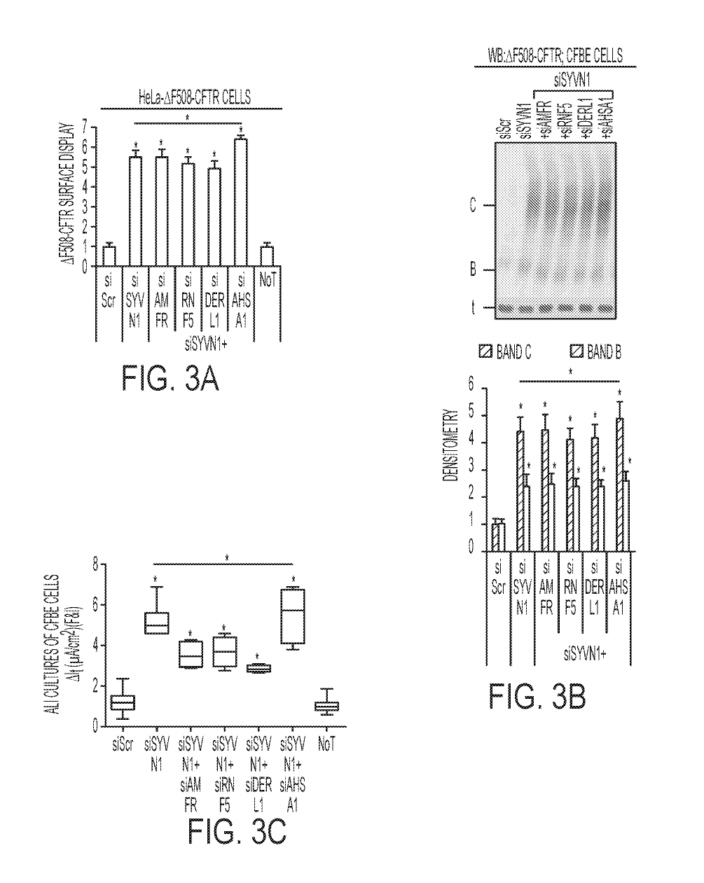

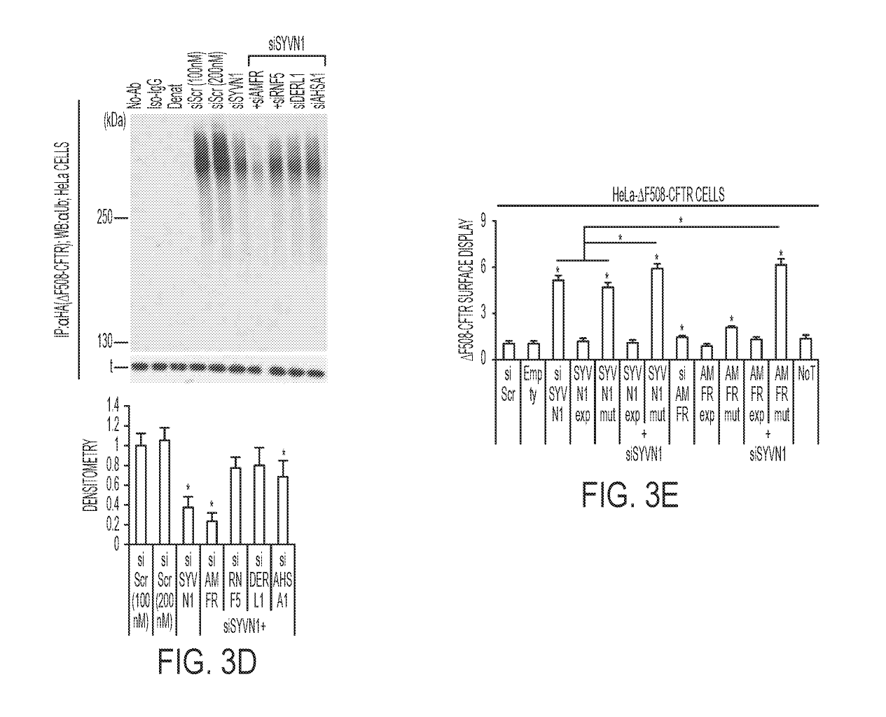

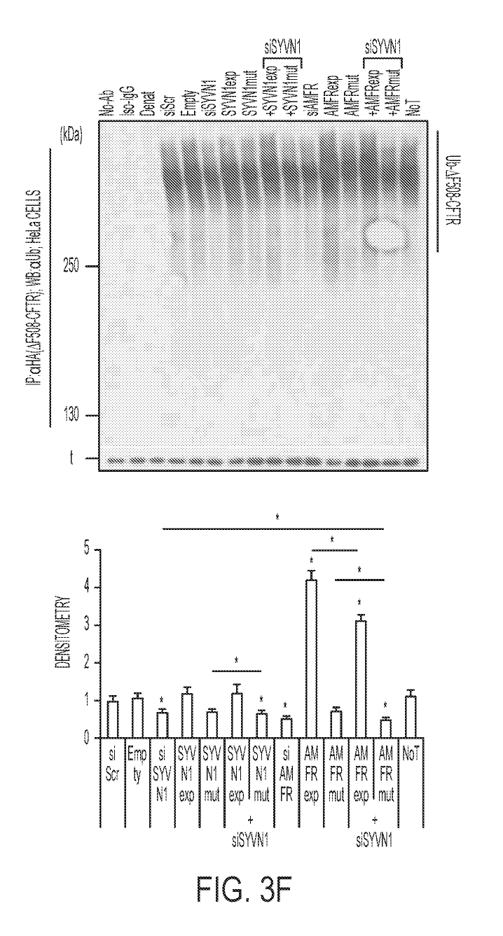

Supplementary FIG. 3. NEDD8 is upregulated in CF airway epithelia. NEDD8 mRNA levels measured by RT-qPCR in well-differentiated primary airway epithelial cultures. Pig CF (ΔF508/ΔF508) and non-CF: n=8 donors; Human CF (ΔF508/ΔF508) and non-CF: n=6 donors. Error bars indicate standard error; statistical significance determined by Student's t-test; **P<0.01, ***P<0.001.

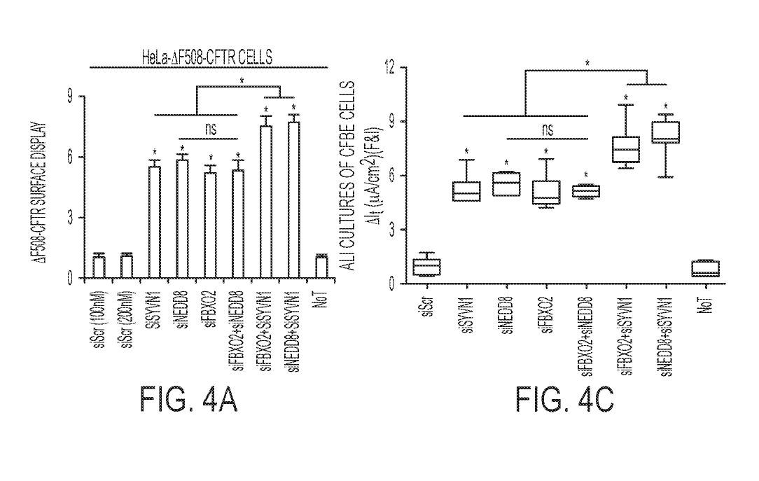

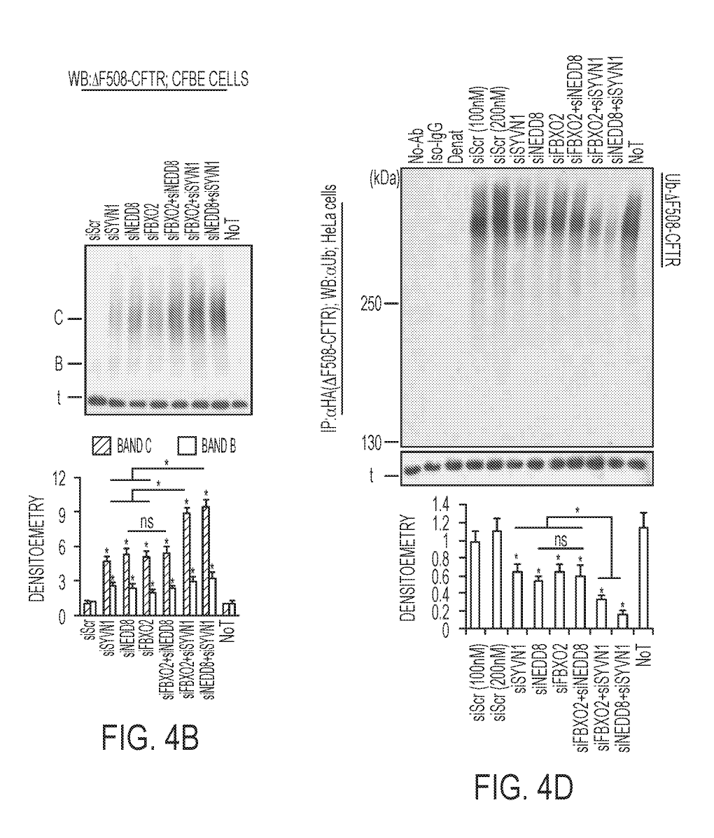

Supplementary FIG. 4. DsiRNA dose-response against genes in the ubiquitin-proteasome system. Remaining mRNA levels of noted genes, relative to the siScr control (at same dose) in CFBE cells, measured by RT-qPCR 24 hr post-transfection. n=4. Representative immunoblot depicting protein levels of noted genes in CFBE cells. Protein harvested 72 hr post-transfection. Error bars indicate standard error; statistical significance determined by the Holm-Bonferroni method; *P<0.05.

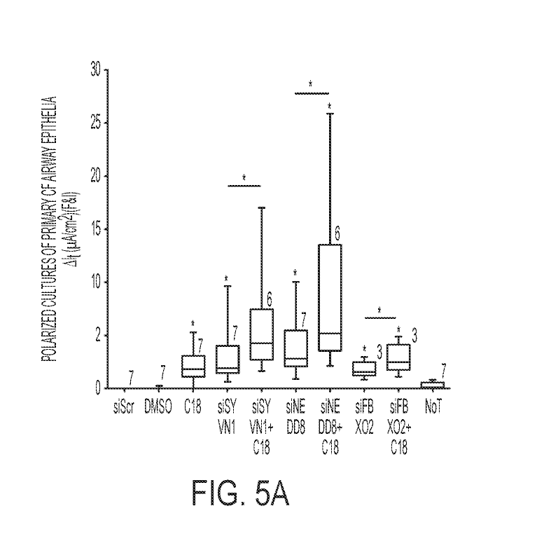

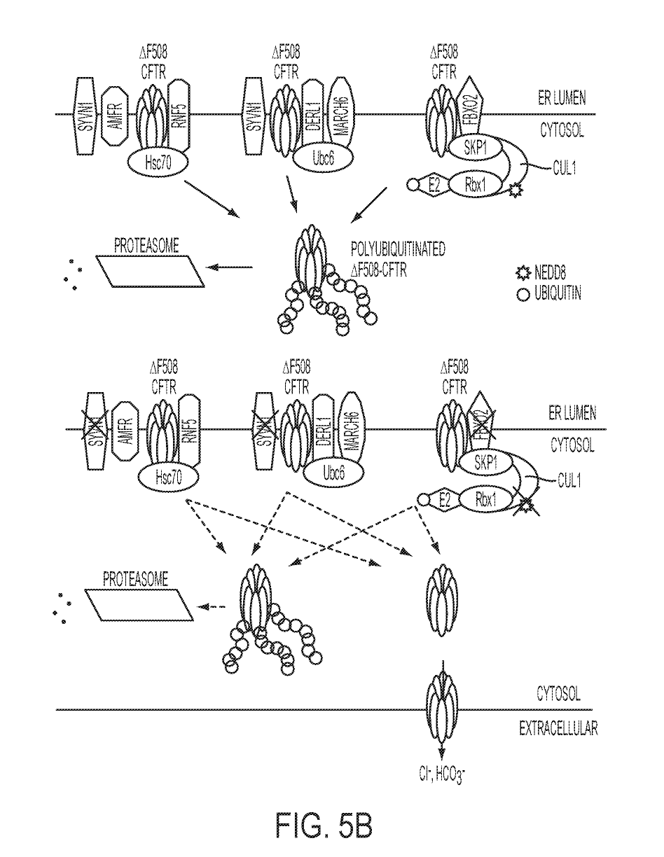

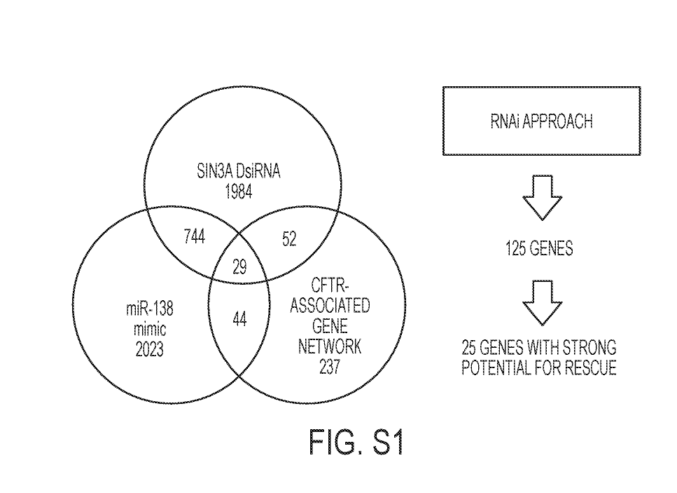

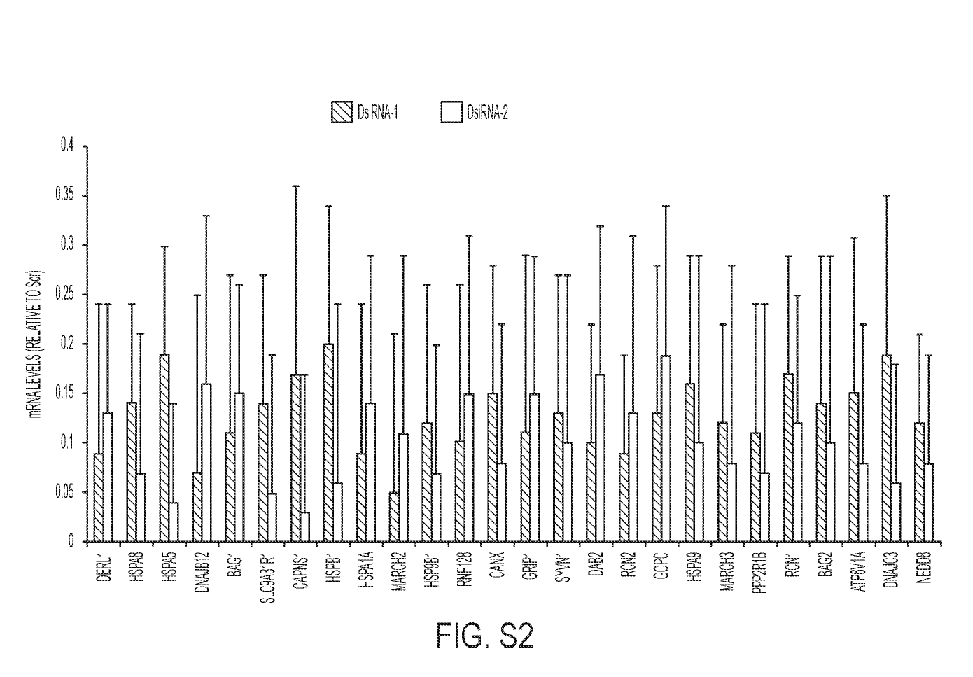

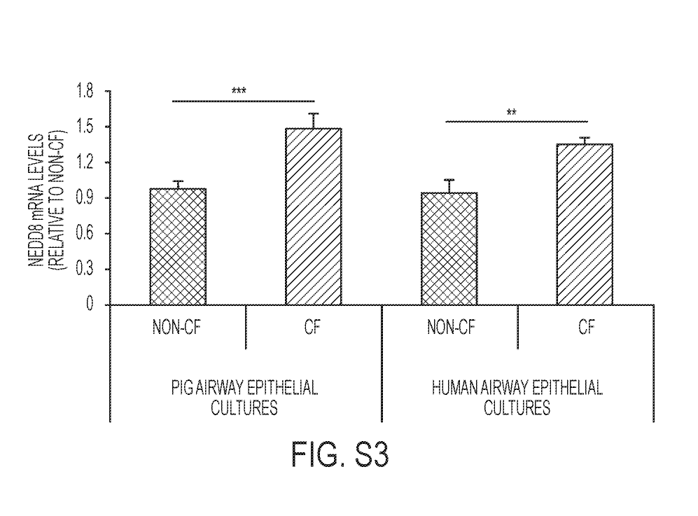

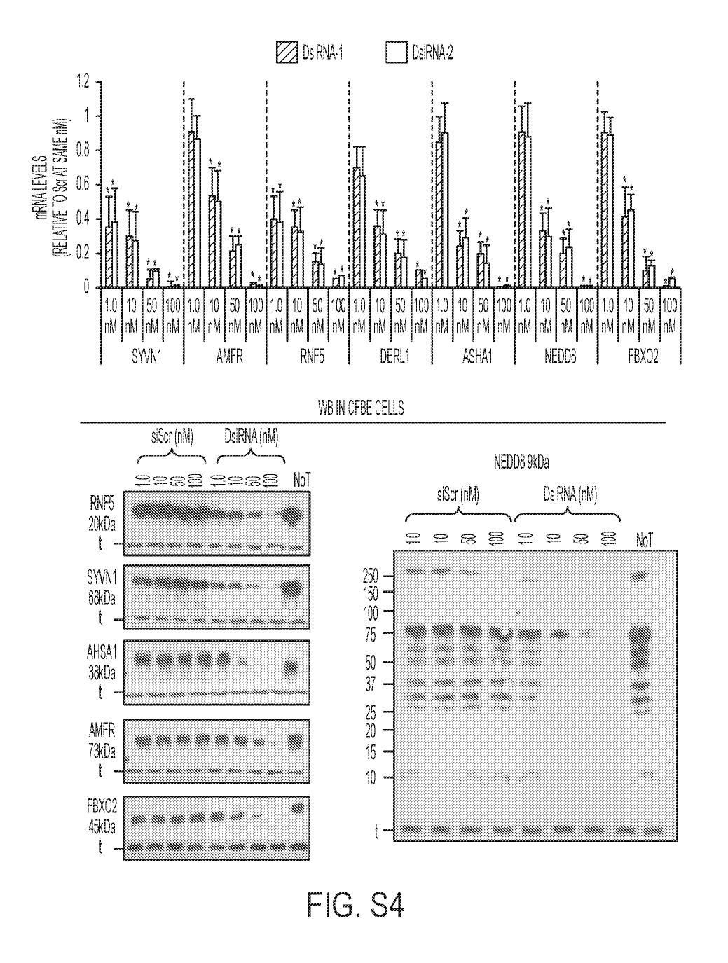

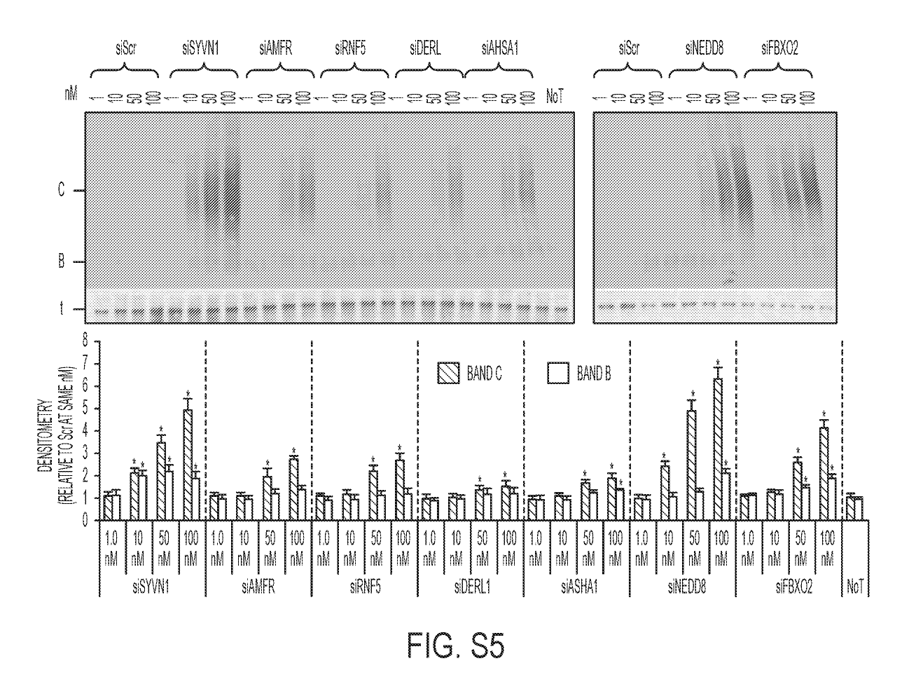

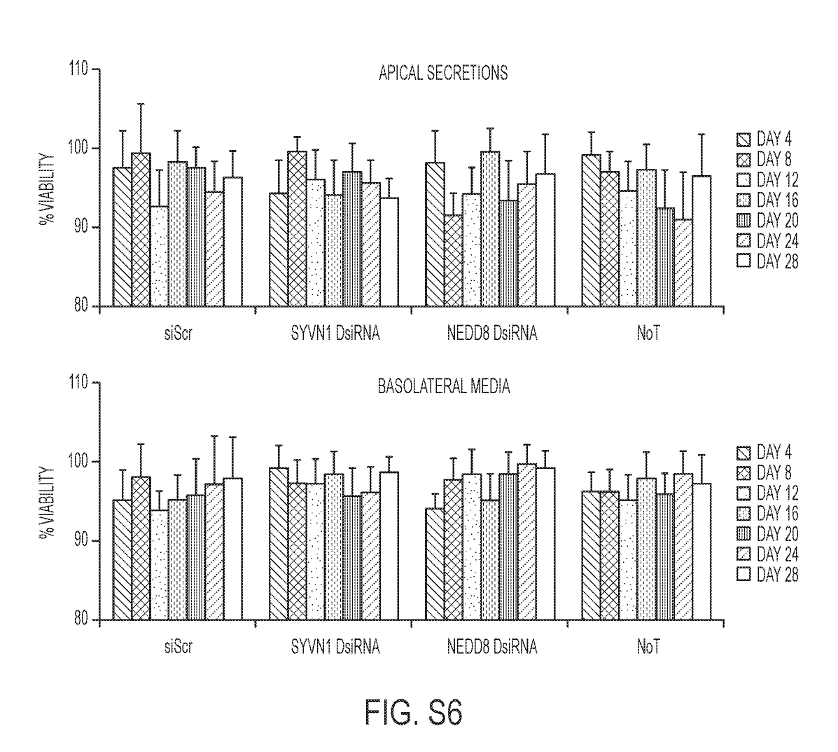

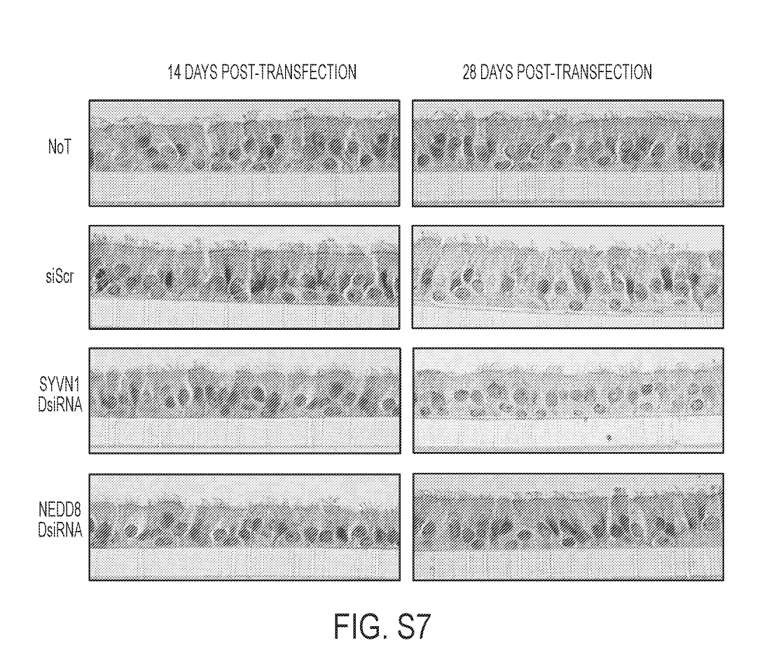

Supplementary FIG. 5. Rescue of ΔF508-CFTR maturation upon inhibition of genes in the ubiquitin-proteasome system. Representative immunoblot depicting ΔF508-CFTR expression in CFBE cells. C=band C, B=band B, t=α-tubulin. Protein harvested 72 hr post-treatment. Densitometry representing fold increase of ΔF508-CFTR bands C and B relative to siScr in CFBE cells. n=3. Error bars indicate standard error; statistical significance determined by the Holm-Bonferroni method; *P<0.05.

Supplementary FIG. 6. LDH release assay upon inhibition of SYVN1 and NEDD8 expression. LDH levels measured in the airway surface liquid and basolateral media of primary air-liquid interface non-CF airway epithelial cultures every 4 days for a period of 28 days post-transfection with noted reagents. n=3 donors (3 cultures per donor).

Supplementary FIG. 7. Cell morphology of primary airway epithelia remains similar after SYVN1 and NEDD8 inhibition. Cell morphology was assessed by hematoxylin and eosin (H&E) staining on primary non-CF airway epithelial cultures at days 14 and 28 post-transfection with noted reagents. n=3 donors (3 cultures per donor).

### DETAILED DESCRIPTION OF THE INVENTION

The most common CFTR mutation, ΔF508, results in protein misfolding and increased proteosomal degradation via Endoplasmic Reticulum-Associated Degradation (ERAD). If ΔF508-CFTR trafficks to the cell membrane, the mutant protein retains partial channel function; motivating therapeutic strategies that can either divert more CFTR away from the ERAD pathway, or enhance stability or activity of ΔF508-CFTR at the cell surface. Delivery of a microRNA (miR)-138 mimic or siRNA against SIN3A to cultured CF airway epithelia increased ΔF508-CFTR mRNA and protein abundance, and partially restored cAMP-stimulated Cl⁻ conductance (WO 2013/119705). The inventors dissected the miR-138/SIN3A regulated gene network to identify individual gene products contributing to the rescue of ΔF508-CFTR function. This network includes 773 genes whose expression is altered in Calu-3 epithelia treated with the miR-138 mimic or the SIN3A siRNA. Within this network, the inventors found that RNA interference (RNAi)-mediated depletion of the ubiquitin ligase SYVN1 or the ubiquitin/proteasome system-regulator, NEDD8 partially restored ΔF508-CFTR-mediated Cl⁻ transport in primary cultures of human CF airway epithelia. Furthermore, in combination with either corrector compound 18 or low temperature, depletion of SYVN1 or NEDD8 dramatically potentiated rescue of ΔF508-CFTR biosynthesis. These results provide new knowledge of the CFTR biosynthetic pathway. Candidates identified using this approach represent new targets for CF therapies.

In certain embodiments, the present invention provides methods of using therapeutic agents to treat cystic fibrosis. In certain embodiments, the present invention provides a method of reducing ΔF508-CFTR ubiquitination or degradation, or increasing ΔF508-CFTR processing or function in a CF cell comprising contacting the cell with a NEDD8 therapeutic agent that inhibits NEDD8 expression in the cell. In certain embodiments, the method further comprises contacting the cell with a FBXO2 therapeutic agent that inhibits FBXO2 expression in the cell. In certain embodiments, the method further comprises contacting the cell with a SYVN1 therapeutic agent that inhibits SYVN1 expression in the cell. In certain embodiments, the method involves inhibiting NEDD8 and FBXO2; in certain embodiments, the method involves inhibiting NEDD8, FBXO2, and SYVN1 expression in the cell.

In certain embodiments, the present invention provides methods of using therapeutic agents to treat cystic fibrosis. In certain embodiments, the present invention provides a method of reducing ΔF508-CFTR ubiquitination or degradation, or increasing ΔF508-CFTR processing or function in a CF cell comprising contacting the cell with a FBXO2 therapeutic agent that inhibits FBXO2 expression in the cell. In certain embodiments, the method further comprises contacting the cell with a SYVN1 therapeutic agent that inhibits SYVN1 expression in the cell.

In certain embodiments, the NEDD8, FBXO2, and/or SYVN1 is inhibited by at least about 10%, 20%, 30%, 40%, 50%, 60%, 70%, 80%, 90% 95%, or 99% as compared to untreated ΔF508-CFTR.

In certain embodiments, the present invention provides methods of reducing ΔF508-CFTR ubiquitination or increasing ΔF508-CFTR processing and function in a CF cell comprising contacting the cell with a SYVN1 therapeutic agent that inhibits SYVN1 and a AHSA1 therapeutic agent that inhibits AHSA1 expression in the cell.

In certain embodiments, the present invention provides methods of reducing ΔF508-CFTR ubiquitination or degradation, or increasing membrane stability of ΔF508-CFTR in a Cystic Fibrosis (CF) cell comprising contacting the cell with (a) a therapeutic agent that inhibits SYVN1 expression in the cell and (b) a CFTR corrector and/or CFTR potentiator.

In certain embodiments, the ΔF508-CFTR function has increased membrane stability. In certain embodiments, the membrane stability is increased by at least about 10%, 20%, 30%, 40%, 50%, 60%, 70%, 80%, 90% 95%, or 99% as compared to untreated ΔF508-CFTR.

In certain embodiments, the ΔF508-CFTR biosynthesis is increased by proteasome inhibition. In certain embodiments, the proteasome is inhibited by at least about 10%, 20%, 30%, 40%, 50%, 60%, 70%, 80%, 90% 95%, or 99% as compared to an untreated CF cell.

In certain embodiments, ΔF508-CFTR ubiquitination is reduced. In certain embodiments, the ΔF508-CFTR ubiquitination is decreased by at least about 10%, 20%, 30%, 40%, 50%, 60%, 70%, 80%, 90% 95%, or 99% as compared to untreated ΔF508-CFTR.

In certain embodiments, the ΔF508-CFTR function in primary airway epithelial cultures is partially restored. In certain embodiments, the ΔF508-CFTR function in primary airway epithelial cultures is restored at least about 10%, 20%, 30%, 40%, 50%, 60%, 70%, 80%, 90% 95%, or 99% as compared to untreated ΔF508-CFTR.

In certain embodiments, the cell is a primary airway epithelial cell. In certain embodiments, the cell is in vivo.

In certain embodiments, the ΔF508-CFTR mediated Cl⁻ transport is improved by at least 10%. In certain embodiments, the ΔF508-CFTR mediated Cl⁻ transport is improved by at least about 10%, 20%, 30%, 40%, 50%, 60%, 70%, 80%, 90% 95%, or 99% as compared to untreated ΔF508-CFTR.

NEDD8 Therapeutic Agents

In certain embodiments, the NEDD8 therapeutic agent is an siRNA oligonucleotide, an ASO oligonucleotide, a small molecule inhibitor, or other chemical inhibitor.

In certain embodiments, the NEDD8 therapeutic agent is a DsiRNA. In certain embodiments, the DsiRNA is one of the following:

| | DsiRNA | Antisense | Sense |
|---|---|---|---|
| Seq Name | # | Strand Sequence | Strand Sequence |
| NEDD8 | 1 | /5Phos/rCrGrUrCrUrUrCrAr | /5Phos/rGrArArGrArUrGrCrUrA |
| | | CrUmUrUmArArUrUrArGr | rArUrUrArArArGrUrGrArArGrA |
| | | CrAmUrCmUrUmCmUmU | CG (SEQ ID NO: 7) |
| | | (SEQ ID NO: 6) | |
| NEDD8 | 2 | /5Phos/rGrUrCrArArUrCrUr | /5Phos/rGrArCrCrGrGrArArArG |
| | | CrAmArUmCrUrCrCrUrUr | rGrArGrArUrUrGrArGrArUrUrG |
| | | UrCmCrGmGrUmCmAmG | AC (SEQ ID NO: 9) |
| | | (SEQ ID NO: 8) | |
| NEDD8 | 3 | /5Phos/rUrCrCrCrUrCrUrUr | /5Phos/rGrGrArGrCrGrUrGrUrG |
| | | UrCmUrCmCrUrCrCrArCrA | rGrArGrGrArGrArArArGrArGrG |
| | | rCmGrCmUrCmCmUmU | GA (SEQ ID NO: 11) |
| | | (SEQ ID NO: 10) | |

r = RNA; m = 2′OMe modification

In certain embodiments, the NEDD8 therapeutic agent is a chemical inhibitor, for example MLN4924 (Soucy et al., “An inhibitor of NEDD8-activating enzyme as a new approach to treat cancer,” Nature (2009) 458(7239):732-736)); 6,6″-biapigenin (Leung et al., “A natural product-like inhibitor of NEDD8-activating enzyme,” Chem Commun (Camb). 2011 Mar. 7; 47(9):2511-3); or piperacillin (Zhong et al., “Structure-based repurposing of FDA-approved drugs as inhibitors of NEDD8-activating enzyme,” Biochimie. 2014 July; 102:211-5).

FBXO2 Therapeutic Agents

In certain embodiments, the FBXO2 therapeutic agent is an siRNA oligonucleotide, an ASO oligonucleotide, a small molecule inhibitor, or other chemical inhibitor.

In certain embodiments, the FBXO2 therapeutic agent is a DsiRNA. In certain embodiments, the DsiRNA is one of the following:

| | DsiRNA | Antisense | Sense |
|---|---|---|---|
| Seq Name | # | Strand Sequence | Strand Sequence |
| FBXO2 | 1 | /5Phos/rGrGrArCrGrCrUrAr | /5Phos/rGrGrCrCrUrUrArArCrUr |
| | | UrGmGrAmCrUrArArGrUr | UrArGrUrCrCrArUrArGrCrGrU |
| | | UrAmArGmGrCmCmUmA | CC (SEQ ID NO: 13) |
| | | (SEQ ID NO: 12) | |
| FBXO2 | 2 | /5Phos/rUrCrArCrGrCrCrCr | /5Phos/ArGrArArUrGrUrArGrAr |
| | | UrCmArCmGrGrArUrCrUr | UrCrCrGrUrGrArGrGrGrCrGrU |
| | | ArCmArUmUrCmUmAmG | GA (SEQ ID NO: 15) |
| | | (SEQ ID NO: 14) | |
| FBXO2 | 3 | /5Phos/rCrArCrGrUrUrCrUr | /5Phos/rGrCrUrArCrUrGrUrCrCr |
| | | CrGmUrGmCrUrCrGrGrAr | GrArGrCrArCrGrArGrArArCrG |
| | | CrAmGrUmArGmCmUmU | TG (SEQ ID NO: 17) |
| | | (SEQ ID NO: 16) | |

r = RNA; m = 2′OMe modification

SYVN1 Therapeutic Agents

In certain embodiments, the SYVN1 therapeutic agent is an siRNA oligonucleotide, an ASO oligonucleotide, a small molecule inhibitor, or other chemical inhibitor.

In certain embodiments, the SYVN1 therapeutic agent is a DsiRNA. In certain embodiments, the DsiRNA is one of the following:

| | DsiRNA | Antisense | Sense |
|---|---|---|---|
| Seq Name | # | Strand Sequence | Strand Sequence |
| SYVN1 | 1 | /5Phos/rGrUrGrGrGrCrCrAr | /5Phos/rGrCrUrArUrGrArArCrU |
| | | GrCmGrAmGrCrArArGrUr | rUrGrCrUrCrGrCrUrGrGrCrCrC |
| | | UrCmArUmArGmCmUmU | AC (SEQ ID NO: 19) |
| | | (SEQ ID NO: 18) | |
| SYVN1 | 2 | /5Phos/rUrCrArUrCrUrGrAr | /5Phos/rArGrUrUrGrUrUrGrGrA |
| | | ArAmCrUmGrUrCrUrCrCr | rGrArCrArGrUrUrUrCrArGrArU |
| | | ArAmCrAmArCmUmCmU | GA (SEQ ID NO: 21) |
| | | (SEQ ID NO: 20) | |
| SYVN1 | 1 | /5Phos/rGrUrGrGrGrCrCrAr | /5Phos/rGrCrUrArUrGrArArCrU |
| 3′UTR | | GrCmGrAmGrCrArArGrUr | rUrGrCrUrCrGrCrUrGrGrCrCrC |
| | | UrCmArUmArGmCmUmU | AC (SEQ ID NO: 23) |
| | | (SEQ ID NO: 22) | |
| SYVN1 | 1 | /5Phos/rGrUrGrArGrGrUrAr | /5Phos/rUrGrCrUrGrCrArGrArU |
| CDS | | CrUmGrGmUrUrGrArUrCr | rCrArArCrCrArGrUrArCrCrUC |
| | | UrGmCrAmGrCmAmUmG | AC (SEQ ID NO: 25) |
| | | (SEQ ID NO: 24) | |

r = RNA; m = 2′OMe modification

In certain embodiments, the SYVN1 therapeutic agent is a chemical inhibitor, such as LS-101 and LS-102 (Yagishita et al., “RING-finger type E3 ubiquitin ligase inhibitors as novel candidates for the treatment of rheumatoid arthritis,” Int J Mol Med. 2012 December; 30(6):1281-6).

AHSA1 Therapeutic Agent

In certain embodiments, the AHSA1 therapeutic agent is an siRNA oligonucleotide, an ASO oligonucleotide, a small molecule inhibitor, or other chemical inhibitor.

In certain embodiments, the AHSA1 therapeutic agent is a DsiRNA. In certain embodiments, the DsiRNA is one of the following:

| Seq | DsiRNA | Antisense | Sense |
|---|---|---|---|
| Name | # | Strand Sequence | Strand Sequence |
| AHSA1 | 1 | /5Phos/rCrCrArCrArUrGrUr | /5Phos/rGrGrArGrUrArCrArArU |
| | | CrCmUrUmUrGrUrArUrUr | rArCrArArArGrGrArCrArUrGrU |
| | | GrUmArCmUrCmCmUmG | GG (SEQ ID NO: 27) |
| | | (SEQ ID NO: 26) | |

r = RNA; m = 2′OMe modification

In certain embodiments, the method further comprises contacting the cell with a CFTR corrector and/or CFTR potentiator. Correctors overcome defective protein processing that normally results in the production of misfolded CFTR. This allows increased trafficking of CFTR to the plasma membrane. In certain embodiments, the CFTR corrector is a “proteostasis inhibitor.” CFTR correctors are compounds that modulate the cellular machineries responsible for folding, degradation and vesicular trafficking. Potentiators increase the activity of defective CFTR at the cell surface. Potentiators can either act on gating defects or conductance defects.

CFTR Correctors

In certain embodiments, the CFTR corrector is a small molecule. Examples of CFTR Correctors include the following small molecule correctors:

| | Other | |
|---|---|---|
| Corrector | Name | Chemical Name |
| C1 | | 6-(1H-Benzoimidazol-2-ylsulfanylmethyl)-2-(6-methoxy-4- |
| | | methyl-quinazolin-2-ylamino)-pyrimidin-4-ol |
| C2 | VRT-640 | 2-{1-[4-(4-Chloro-benzensulfonyl)-piperazin-1-yl]-ethyl}-4- |
| | | piperidin-1-yl-quinazoline |
| C3 | VTR-325 | 4-Cyclohexyloxy-2-{1-[4-(4-methoxy-benzensulfonyl)- |
| | | piperazin-1-yl]-ethyl}-quinazoline |
| C4 | Corr-4a | N-[2-(5-Chloro-2-methoxy-phenylamino)-4′-methyl- |
| | | [4,5′]bithiazolyl-2′-yl]-benzamide |
| C5 | Corr-5a | 4,5,7-trimethyl-N-phenylquinolin-2-amine |
| C6 | Corr5c | N-(4-bromophenyl)-4-methylquinolin-2-amine |
| C7 | Genzyme | 2-(4-isopropoxypicolinoyl)-N-(4-pentylphenyl)-1,2,3,4- |
| | cmpd 48 | tetrahydroisoquinoline-3-carboxamide |
| C8 | | N-(2-fluorophenyl)-2-(1H-indol-3-yl)-2-oxoacetamide |
| C9 | KM111060 | 7-chloro-4-(4-(4-chlorophenylsulfonyl)piperazin-1-yl)quinoline |
| C11 | Dynasore | (Z)-N′-(3,4-dihydroxybenzylidene)-3-hydroxy-2- |
| | | naphthohydrazide |
| C12 | Corr-2i | N-(4-fluorophenyl)-4-p-tolylthiazol-2-amine |
| C13 | Corr-4c | N-(2-(3-acetylphenylamino)-4′-methyl-4,5′-bithiazol-2′- |
| | | yl)benzamide |
| C14 | Corr-4d | N-(2′-(2-methoxyphenylamino)-4-methyl-5,5′-bithiazol-2- |
| | | yl)benzamide |
| C15 | Corr-2b | N-phenyl-4-(4-vinylphenyl)thiazol-2-amine |
| C16 | Corr-3d | 2-(6-methoxy-4-methylquinazolin-2-ylamino)-5,6- |
| | | dimethylpyrimidin-4(1H)-one |
| C17 | 15jf | N-(2-(5-chloro-2-methoxyphenylamino)-4′-methyl-4,5′- |
| | | bithiazol-2′-yl)pivalamide |
| C18 | CF-106951 | 1-(benzo[d][1,3]dioxol-5-yl)-N-(5-((2-chlorophenyl)(3- |
| | | hydroxypyrrolidin-1-yl)methyl)thiazol-2- |
| | | yl)cyclopropanecarboxamide |
| VX-809 | Lumacaftor | 3-{6-{[1-(2,2-difluoro-1,3-benzodioxol-5- |
| | | yl)cyclopropanecarbonyl]amino}-3-methylpyridin-2-yl}benzoic |
| | | acid |
| Core-cor-II | | |
| RDR1 | | |
| RDR2 | | |
| RDR3 | | |
| Co-Po-22 | | |
| Vx-661 | | |
| Vx-325 | | |
| Vx-422 | | |
| Vx-532 | | |

In certain embodiments, the CFTR corrector is a chemical chaperone. In certain embodiments, the chemical chaperone is glycerol, TMAO (Trimethylamine N-oxide), taurine, myo-inositol and/or D-sorbitol.

CFTR Potentiator

In certain embodiments, the CFTR potentiator is VX-770 (Kalydeco).

Auxiliary Compounds

In certain embodiments, the present invention provides additionally contacting the cell with an auxiliary compound. Examples of auxiliary compounds include the following:

| Drug (alternative | | |
|---|---|---|
| name) | Developers | Modes of action |
| Bronchitol | Central Sydney Area | Osmotic agent |
| | Health | |
| | Service/Pharmaxis | |
| Ataluren (Translarna) | PTC Therapeutics | Facilitates read-through of stop- |
| | | codons |
| CFTR gene therapy | CFGTC | Gene therapy |
| N-6022 | N30 Pharmaceuticals | GSNOR inhibitor |
| Lynovex (NM-001) | NovaBiotics | Antibacterial, mucolytic |
| OligoG | AlgiPharma | Antibiotic oligosaccharide |
| Alpha-1 antitrypsin | Grifols | Anti-inflammatory, proteinase |
| | | inhibitor |
| KB001-A | KaloBios | Anti-inflammatory, monoclonal |
| | Pharmaceuticals/CFF | Fab fragment |
| Sildenafil (Revatio) | CFF | Anti-inflammatory, |
| | | phosphodiesterase inhibitor |
| Levofloxacin (Aeroquin | Aptalis Pharma/CFF | Anti-infective |
| or MP-376) | | |
| Arikace (inhaled | Insmed/CFF | Anti-infective |
| amikacin) | | |
| AeroVanc (inhaled | Savara | Anti-infective |
| vancomycin) | Pharmaceuticals/CFF | |
| Liprotamase | Eli Lilly | PERT |

SIN3A Therapeutic Agents

In certain embodiments, the further comprises contacting the cell with a therapeutic agent, wherein the agent comprises miR-138, a miR-138 mimic, an anti-SIN3A RNAi molecule, and/or an anti-SIN3A antisense oligonucleotide (ASO) or other agent that suppresses SIN3A expression, a small molecule drug that interferes with SIN3A activity or whose actions mimic the biological effects of SIN3A suppression.

1. pre-miR-138 and miR-138:

Pre-miR-138:

hsa-mir-138-1 MI0000476
(SEQ ID NO: 1)
CCCUGGCAUGGUGUGGUGGGGCAGCUGGUGUUGUGAAUCAGGCCGUUGCC
AAUCAGAGAACGGCUACUUCACAACACCAGGGCCACACCACACUACAGG
hsa-mir-138-2 MI0000455
(SEQ ID NO: 2)
CGUUGCUGCAGCUGGUGUUGUGAAUCAGGCCGACGAGCAGCGCAUCCUCU
UACCCGGCUAUUUCACGACACCAGGGUUGCAUCA

Mature miRNA:

hsa-mir-138-5p
(SEQ ID NO: 3)
AGCUGGUGUUGUGAAUCAGGCCG
miR-138 mimic-
Sense strand sequence:
(SEQ ID NO: 4)
/5SpC3/rCmG rGmC/iSpC3/ mUrGmA rUmUrC mArCmA rAmCrA
mCrCmA rGmCrU
Antisense strand sequence:
(SEQ ID NO: 5)
/5Phos/rArG rCrUrG rGrUrG rUrUrG rUrGrA rArUrC
rArGrG mCmCmG

As used herein “5SpC3” and “iSpC3” represent propanediol groups (e.g., a “C3 spacer”), rN represent RNA bases, mN represent 2′OMe RNA bases, and 5Phos represents a 5′-phosphate group. For example, as used herein, the designation “ACGU” and “rA rC rG rU” are equivalent. In certain embodiments, a miR-138 mimic is a synthetic nucleic acid which shows miR-138-like activity in a mammalian cell following transfection. In certain embodiments this is a long pri-miRNA, a shorter pre-miRNA (as shown above), the even shorter mature miRNA, or a modified compound which has been optimized to improve performance (as shown above). Many different miR mimics can be designed. The one above was employed in the present studies and is suitable for use as an example but in no way should be restrictive of the wider body of nucleic acid compositions that can be employed as a miR-138 mimic.

CFTR Small Molecule Therapeutic Agents

2. Aminoglutethimide: (RS)-3-(4-aminophenyl)-3-ethyl-piperidine-2,6-dione

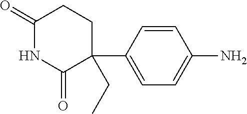 [SMILES: CCC1(c2ccc(N)cc2)CCC(=O)NC1=O]

3. Biperiden: (1RS,2SR,4RS)-1-(bicyclo[2.2.1]hept-5-en-2-yl)-1-phenyl-3-(piperidin-1-yl)propan-1-ol

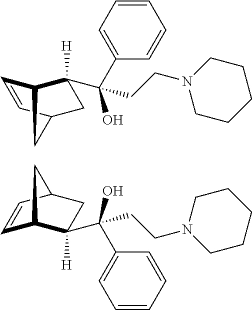

4. Diphenhydramine

5. Rottlerin: 3′-[(8-Cinnamoyl-5,7-dihydroxy-2,2-dimethyl-2H-1-benzopyran-6-yl)methyl]-2′,4′,6′-trihydroxy-5′-methylacetophenone

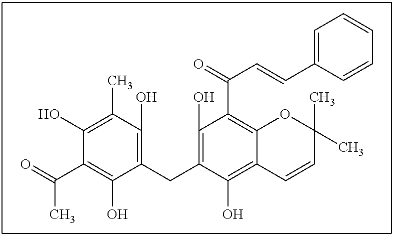 [SMILES: CC(=O)c1c(O)c(C)c(O)c(Cc2c(O)c3c(c(C(=O)/C=C/c4ccccc4)c2O)OC(C)(C)C=C3)c1O]

6. Midodrine: (RS)—N-[2-(2,5-dimethoxyphenyl)-2-hydroxyethyl]glycinamide

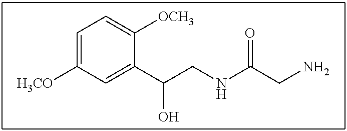 [SMILES: COc1ccc(OC)c(C(O)CNC(=O)CN)c1]

7. Thioridazine: 10-{2-[(RS)-1-Methylpiperidin-2-yl]ethyl}-2-methylsulfanylphenothiazine

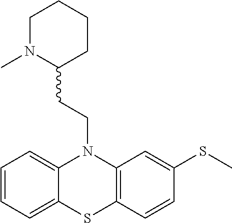 [SMILES: CSc1ccc2c(c1)N(CCC1CCCCN1C)c1ccccc1S2]

8. Sulfadimethoxine: 4-amino-N-(2,6-dimethoxypyrimidin-4-yl)benzenesulfonamide

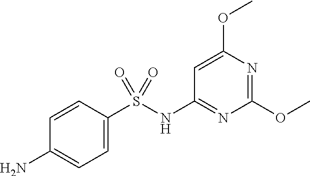 [SMILES: COc1cc(NS(=O)(=O)c2ccc(N)cc2)nc(OC)n1]

9. Neostigmine: 3-{[(dimethylamino)carbonyl]oxy}-N,N,N-trimethylbenzenaminium

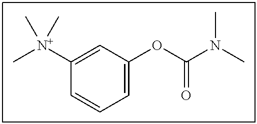

10. Pyridostigmine: 3-[(dimethylcarbamoyl)oxy]-1-methylpyridinium

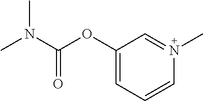

11. Pizotifen: 4-(1-methyl-4-piperidylidine)-9,10-dihydro-4H-benzo-[4,5]cyclohepta[1,2]-thiophene

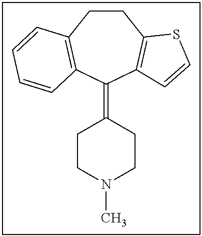 [SMILES: CN1CCC(=C2c3ccccc3CCc3sccc32)CC1]

12. Tyrophostin (AG-1478): N-(3-chlorophenyl)-6,7-dimethoxy-4-quinazolinamine

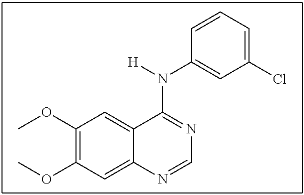 [SMILES: COc1cc2ncnc(Nc3cccc(Cl)c3)c2cc1OC]

13. Valproic Acid: 2-propylpentanoic acid

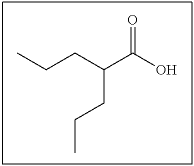

14. Scriptaid: N-Hydroxy-1,3-dioxo-1H-benz[de]isoquinoline-2(3H)-hexanamide

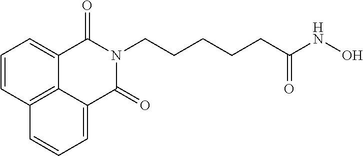 [SMILES: O=C(CCCCCN1C(=O)c2cccc3cccc(c23)C1=O)NO]

15. Neomycin: O-2,6-diamino-2,6-dideoxy-α-D-glucopyranosyl(1→3)-O-β-D-ribofuranosyl-(1→5) O-[2,6-diamino-2,6-dideoxy-α-D-glucopyranosyl-(1→4)]-2-deoxy-D-streptamine

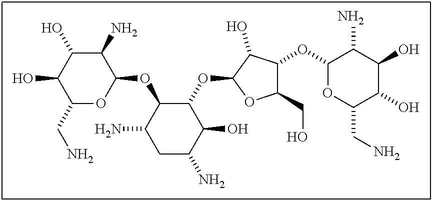

In certain embodiments, pharmaceutically acceptable salts of these compounds are used. For in vivo use, a therapeutic compound as described herein is generally incorporated into a pharmaceutical composition prior to administration. Within such compositions, one or more therapeutic compounds as described herein are present as active ingredient(s) (i.e., are present at levels sufficient to provide a statistically significant effect on the symptoms of cystic fibrosis, as measured using a representative assay). A pharmaceutical composition comprises one or more such compounds in combination with any pharmaceutically acceptable carrier(s) known to those skilled in the art to be suitable for the particular mode of administration. In addition, other pharmaceutically active ingredients (including other therapeutic agents) may, but need not, be present within the composition.

RNA Interference (RNAi) Molecules

“RNA interference (RNAi)” is the process of sequence-specific, post-transcriptional gene silencing initiated by a small interfering RNA (siRNA). During RNAi, siRNA induces degradation of target mRNA with consequent sequence-specific inhibition of gene expression.

An “RNA interference,” “RNAi,” “small interfering RNA” or “short interfering RNA” or “siRNA” or “short hairpin RNA” or “shRNA” molecule, or “miRNA” is a RNA duplex of nucleotides that is targeted to a nucleic acid sequence of interest, for example, SIN3A. As used herein, the term “siRNA” is a generic term that encompasses all possible RNAi triggers. An “RNA duplex” refers to the structure formed by the complementary pairing between two regions of a RNA molecule. siRNA is “targeted” to a gene in that the nucleotide sequence of the duplex portion of the siRNA is complementary to a nucleotide sequence of the targeted gene. In certain embodiments, the siRNAs are targeted to the sequence encoding SIN3A. In some embodiments, the length of the duplex of siRNAs is less than 30 base pairs. In some embodiments, the duplex can be 32, 31, 30, 29, 28, 27, 26, 25, 24, 23, 22, 21, 20, 19, 18, 17, 16, 15, 14, 13, 12, 11 or 10 base pairs in length. In some embodiments, the length of the duplex is 19 to 32 base pairs in length. In certain embodiment, the length of the duplex is 19 or 21 base pairs in length. The RNA duplex portion of the siRNA can be part of a hairpin structure. In addition to the duplex portion, the hairpin structure may contain a loop portion positioned between the two sequences that form the duplex. The loop can vary in length. In some embodiments the loop is 5, 6, 7, 8, 9, 10, 11, 12, 13, 14, 15, 16, 17, 18, 19, 20, 21, 22, 23, 24, 25, 26 or 27 nucleotides in length. In certain embodiments, the loop is 18 nucleotides in length. The hairpin structure can also contain 3′ and/or 5′ overhang portions. In some embodiments, the overhang is a 3′ and/or a 5′ overhang 0, 1, 2, 3, 4 or 5 nucleotides in length.

As used herein, Dicer-substrate RNAs (DsiRNAs) are chemically synthesized asymmetric 25-mer/27-mer duplex RNAs that have increased potency in RNA interference compared to traditional siRNAs. Traditional 21-mer siRNAs are designed to mimic Dicer products and therefore bypass interaction with the enzyme Dicer. Dicer has been recently shown to be a component of RISC and involved with entry of the siRNA duplex into RISC. Dicer-substrate siRNAs are designed to be optimally processed by Dicer and show increased potency by engaging this natural processing pathway. Using this approach, sustained knockdown has been regularly achieved using sub-nanomolar concentrations. (U.S. Pat. No. 8,084,599; Kim et al., Nature Biotechnology 23:222 2005; Rose et al., Nucleic Acids Res., 33:4140 2005).

The transcriptional unit of a “shRNA” is comprised of sense and antisense sequences connected by a loop of unpaired nucleotides. shRNAs are exported from the nucleus by Exportin-5, and once in the cytoplasm, are processed by Dicer to generate functional siRNAs. “miRNAs” stem-loops are comprised of sense and antisense sequences connected by a loop of unpaired nucleotides typically expressed as part of larger primary transcripts (pri-miRNAs), which are excised by the Drosha-DGCR8 complex generating intermediates known as pre-miRNAs, which are subsequently exported from the nucleus by Exportin-5, and once in the cytoplasm, are processed by Dicer to generate functional miRNAs or siRNAs. “Artificial miRNA” or an “artificial miRNA shuttle vector”, as used herein interchangably, refers to a primary miRNA transcript that has had a region of the duplex stem loop (at least about 9-20 nucleotides) which is excised via Drosha and Dicer processing replaced with the siRNA sequences for the target gene while retaining the structural elements within the stem loop necessary for effective Drosha processing. The term “artificial” arises from the fact the flanking sequences (˜35 nucleotides upstream and ˜40 nucleotides downstream) arise from restriction enzyme sites within the multiple cloning site of the siRNA. As used herein the term “miRNA” encompasses both the naturally occurring miRNA sequences as well as artificially generated miRNA shuttle vectors.

The siRNA can be encoded by a nucleic acid sequence, and the nucleic acid sequence can also include a promoter. The nucleic acid sequence can also include a polyadenylation signal. In some embodiments, the polyadenylation signal is a synthetic minimal polyadenylation signal or a sequence of six Ts.

“Off-target toxicity” refers to deleterious, undesirable, or unintended phenotypic changes of a host cell that expresses or contains a siRNA. Off-target toxicity may result in loss of desirable function, gain of non-desirable function, or even death at the cellular or organismal level. Off-target toxicity may occur immediately upon expression of the siRNA or may occur gradually over time. Off-target toxicity may occur as a direct result of the expression siRNA or may occur as a result of induction of host immune response to the cell expressing the siRNA. Without wishing to be bound by theory, off-target toxicity is postulated to arise from high levels or overabundance of RNAi substrates within the cell. These overabundant or overexpressed RNAi substrates, including without limitation pre- or pri RNAi substrates as well as overabundant mature antisense-RNAs, may compete for endogenous RNAi machinery, thus disrupting natural miRNA biogenesis and function. Off-target toxicity may also arise from an increased likelihood of silencing of unintended mRNAs (i.e., off-target) due to partial complementarity of the sequence. Off target toxicity may also occur from improper strand biasing of a non-guide region such that there is preferential loading of the non-guide region over the targeted or guide region of the RNAi. Off-target toxicity may also arise from stimulation of cellular responses to dsRNAs which include dsRNA. “Decreased off target toxicity” refers to a decrease, reduction, abrogation or attenuation in off target toxicity such that the therapeutic effect is more beneficial to the host than the toxicity is limiting or detrimental as measured by an improved duration or quality of life or an improved sign or symptom of a disease or condition being targeted by the siRNA. “Limited off target toxicity” or “low off target toxicity” refer to unintended undesirable phenotypic changes to a cell or organism, whether detectable or not, that does not preclude or outweigh or limit the therapeutic benefit to the host treated with the siRNA and may be considered a “side effect” of the therapy. Decreased or limited off target toxicity may be determined or inferred by comparing the in vitro analysis such as Northern blot or qPCR for the levels of siRNA substrates or the in vivo effects comparing an equivalent shRNA vector to the miRNA shuttle vector of the present invention.

“Knock-down,” “knock-down technology” refers to a technique of gene silencing in which the expression of a target gene is reduced as compared to the gene expression prior to the introduction of the siRNA, which can lead to the inhibition of production of the target gene product. The term “reduced” is used herein to indicate that the target gene expression is lowered by 1-100%. In other words, the amount of RNA available for translation into a polypeptide or protein is minimized. For example, the amount of protein may be reduced by 10, 20, 30, 40, 50, 60, 70, 80, 90, 95, or 99%. In some embodiments, the expression is reduced by about 90% (i.e., only about 10% of the amount of protein is observed a cell as compared to a cell where siRNA molecules have not been administered). Knock-down of gene expression can be directed by the use of RNAi molecules.

According to a method of the present invention, the expression of CF is modified via RNAi. For example, SIN3A expression and/or function is suppressed in a cell. The term “suppressing” refers to the diminution, reduction or elimination in the number or amount of transcripts present in a particular cell. It also relates to reductions in functional protein levels by inhibition of protein translation, which do not necessarily correlate with reductions in mRNA levels. For example, the accumulation of mRNA encoding SIN3A is suppressed in a cell by RNA interference (RNAi), e.g., the gene is silenced by sequence-specific double-stranded RNA (dsRNA), which is also called small interfering RNA (siRNA). These siRNAs can be two separate RNA molecules that have hybridized together, or they may be a single hairpin wherein two portions of a RNA molecule have hybridized together to form a duplex.

A mutant protein refers to the protein encoded by a gene having a mutation, e.g., a missense or nonsense mutation in one or both alleles of a gene, such as CFTR, causing disease. The term “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters. An “allele” is one of several alternative forms of a gene occupying a given locus on a chromosome.

The term “nucleic acid” refers to deoxyribonucleic acid (DNA) or ribonucleic acid

(RNA) and polymers thereof in either single- or double-stranded form, composed of monomers (nucleotides) containing a sugar, phosphate and a base that is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues. A “nucleic acid fragment” is a portion of a given nucleic acid molecule.

A “nucleotide sequence” is a polymer of DNA or RNA that can be single-stranded or double-stranded, optionally containing synthetic, non-natural or altered nucleotide bases capable of incorporation into DNA or RNA polymers.

The terms “nucleic acid,” “nucleic acid molecule,” “nucleic acid fragment,” “nucleic acid sequence or segment,” or “polynucleotide” are used interchangeably and may also be used interchangeably with gene, cDNA, DNA and RNA encoded by a gene.

The invention encompasses isolated or substantially purified nucleic acid nucleic acid molecules and compositions containing those molecules. In the context of the present invention, an “isolated” or “purified” DNA molecule or RNA molecule is a DNA molecule or RNA molecule that exists apart from its native environment and is therefore not a product of nature. An isolated DNA molecule or RNA molecule may exist in a purified form or may exist in a non-native environment such as, for example, a transgenic host cell. For example, an “isolated” or “purified” nucleic acid molecule or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. Fragments and variants of the disclosed nucleotide sequences are also encompassed by the present invention. By “fragment” or “portion” is meant a full length or less than full length of the nucleotide sequence.

“Naturally occurring,” “native,” or “wild-type” is used to describe an object that can be found in nature as distinct from being artificially produced. For example, a protein or nucleotide sequence present in an organism (including a virus), which can be isolated from a source in nature and that has not been intentionally modified by a person in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the invention will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

The terms “protein,” “peptide” and “polypeptide” are used interchangeably herein.

“Functional RNA” refers to sense RNA, antisense RNA, ribozyme RNA, siRNA, or other RNA that may not be translated but yet has an effect on at least one cellular process.

The term “RNA transcript” or “transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell.

“Operably-linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation of an endogenous gene, heterologous gene or nucleic acid segment, or a transgene in cells. For example, in the case of siRNA constructs, expression may refer to the transcription of the siRNA only. In addition, expression refers to the transcription and stable accumulation of sense (mRNA) or functional RNA. Expression may also refer to the production of protein.

The siRNAs of the present invention can be generated by any method known to the art, for example, by in vitro transcription, recombinantly, or by synthetic means. In one example, the siRNAs can be generated in vitro by using a recombinant enzyme, such as T7 RNA polymerase, and DNA oligonucleotide templates.

Modifications of Oligonucleotides

In a preferred aspect, the oligonucleotides of the present invention (e.g., DsiRNAs) are modified to improve stability in serum or growth medium for cell cultures, or otherwise to enhance stability during delivery to subjects and/or cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine, or cytosine by 5′-methylcytosine, can be tolerated without affecting the efficiency of oligonucleotide reagent-induced modulation of splice site selection. For example, the absence of a 2′ hydroxyl may significantly enhance the nuclease resistance of the oligonucleotides in tissue culture medium.

In an embodiment of the present invention the oligonucleotides, e.g., DsiRNAs, may contain at least one modified nucleotide analogue. The nucleotide analogues may be located at positions where the target-specific activity, e.g., the splice site selection modulating activity is not substantially effected, e.g., in a region at the 5′-end and/or the 3′-end of the oligonucleotide molecule. Particularly, the ends may be stabilized by incorporating modified nucleotide analogues.

In certain embodiments, nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In preferred backbone-modified ribonucleotides, the phosphoester group connecting to adjacent ribonucleotides is replaced by a modified group, e.g., of phosphothioate group. In preferred sugar-modified ribonucleotides, the 2′ OH-group is replaced by a group selected from CH₃, H, OR, R, halo, SH, SR, NH₂, NHR, NR₂ or ON, wherein R is C₁-C₆ alkyl, alkenyl or alkynyl and halo is F, Cl, Br or I. In a preferred embodiment, the 2′ OH-group is replaced by CH₃.

Certain embodiments include nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to phosphorothioate derivatives and acridine substituted nucleotides, 2′O-methyl substitutions, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluraci I₅ 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; O- and N-alkylated nucleotides, e.g., N6-methyl adenosine. It should be noted that the above modifications may be combined. Oligonucleotides of the invention also may be modified with chemical moieties (e.g., cholesterol) that improve the in vivo pharmacological properties of the oligonucleotides. Within the oligonucleotides (e.g., oligoribonucleotides) of the invention, as few as one and as many as all nucleotides of the oligonucleotide can be modified. For example, a 20-mer oligonucleotide (e.g., oligoribonucleotide) of the invention may contain 1, 2, 3, 4, 5, 6, 7, 8, 9, 10, 11, 12, 13, 14, 15, 16, 17, 18, 19 or 20 modified nucleotides. In preferred embodiments, the modified oligonucleotides (e.g., oligoribonucleotides) of the invention will contain as few modified nucleotides as are necessary to achieve a desired level of in vivo stability and/or bio-accessibility while maintaining cost effectiveness. A DsiRNA of the invention include oligonucleotides synthesized to include any combination of modified bases disclosed herein in order to optimize function. In one embodiment, a DsiRNA of the invention comprises at least two different modified bases. In another embodiment, a DsiRNA of the invention may comprise alternating 2′O-methyl substitutions and LNA bases.

An oligonucleotide of the invention can be an α-anomeric nucleic acid molecule. An α-anomeric nucleic acid molecule forms specific double-stranded hybrids with complementary RNA in which, contrary to the usual α-units, the strands run parallel to each other (Gaultier et al., 1987, Nucleic Acids Res. 15:6625-6641). The oligonucleotide can also comprise a 2′-O-methylribonucleotide (Inoue et al., 1987, Nucleic Acids Res. 15:6131-6148) or a chimeric RNA-DNA analogue (Inoue et al., 1987, FEBS Lett. 215:327-330).

In various embodiments, the oligonucleotides of the invention can be modified at the base moiety, sugar moiety or phosphate backbone to improve, e.g., the stability, hybridization, or solubility of the molecule. For example, the deoxyribose phosphate backbone of the nucleic acid molecules can be modified to generate peptide nucleic acid molecules (see Hyrup et al., 1996, Bioorganic & Medicinal Chemistry 4(1): 5-23). As used herein, the terms “peptide nucleic acids” or “PNAs” refer to nucleic acid mimics, e.g., DNA mimics, in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al. (1996), supra; Perry-O'Keefe et al. (1996) Proc. Natl. Acad. Sci. USA 93:14670-675. In another embodiment, PNAs can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras can be generated which can combine the advantageous properties of PNA and DNA. Such chimeras allow DNA recognition enzymes, e.g., RNase H and DNA polymerases, to interact with the DNA portion while the PNA portion would provide high binding affinity and specificity. PNA-DNA chimeras can be linked using linkers of appropriate lengths selected in terms of base stacking, number of bonds between the nucleobases, and orientation (Hyrup, 1996, supra). The synthesis of PNA-DNA chimeras can be performed as described in Hyrup (1996), supra, and Finn et al. (1996) Nucleic Acids Res. 24(17):3357-63. For example, a DNA chain can be synthesized on a solid support using standard phosphoramidite coupling chemistry and modified nucleoside analogs. Compounds such as 5′-(4-methoxytrityl)amino-5′-deoxy-thymidine phosphoramidite can be used as a link between the PNA and the 5′ end of DNA (Mag et al, 1989, Nucleic Acids Res. 17:5973-88). PNA monomers are then coupled in a step-wise manner to produce a chimeric molecule with a 5′ PNA segment and a 3′ DNA segment (Finn et al., 1996, Nucleic Acids Res. 24(17): 3357-63). Alternatively, chimeric molecules can be synthesized with a 5′ DNA segment and a 3′ PNA segment (Peterser et al., 1975, Bioorganic Med. Chem. Lett. 5: 1119-11124).

The oligonucleotides of the invention can also be formulated as morpholino oligonucleotides. In such embodiments, the riboside moiety of each subunit of an oligonucleotide of the oligonucleotide is converted to a morpholine moiety (morpholine=C₄H9NO; refer to Heasman, J. 2002 Developmental Biology 243, 209-214, the entire contents of which are incorporated herein by reference).

A further preferred oligonucleotide modification includes Locked Nucleic Acids (LNAs) in which the 2′-hydroxyl group is linked to the 3′ or 4′ carbon atom of the sugar ring thereby forming a bicyclic sugar moiety. The linkage is preferably a methelyne (˜CH₂˜)ₙ group bridging the 2′ oxygen atom and the 4′ carbon atom wherein n is 1 or 2. LNAs and preparation thereof are described in WO 98/39352 and WO 99/14226, the entire contents of which are incorporated by reference herein. In other embodiments, the oligonucleotide can include other appended groups such as peptides (e.g., for targeting host cell receptors in vivo), or agents facilitating transport across the cell membrane (see, e.g., Letsinger et al., 1989, Proc. Natl. Acad. Sci. USA 86:6553-6556; Lemaitre et al., 1987, Proc. Natl. Acad. Sci. USA 84:648-652; PCT Publication No. WO 88/09810) or the blood-brain barrier (see, e.g., PCT Publication No. WO 89/10134). In addition, oligonucleotides can be modified with hybridization-triggered cleavage agents (see, e.g., Krol et al., 1988, Bio/Techniques 6:958-976) or intercalating agents (see, e.g., Zon, 1988, Pharm. Res. 5:539-549). To this end, the oligonucleotide can be conjugated to another molecule, e.g., a peptide, hybridization triggered cross-linking agent, transport agent, hybridization-triggered cleavage agent, etc.

In certain embodiments, the DsiRNA comprises at least one nucleotide that contains a non-naturally occurring modification comprising at least one of a chemical composition of phosphorothioate 2′-O-methyl, phosphorothioate 2′-MOE, locked nucleic acid (LNA) peptide nucleic acid (PNA), phosphorodiamidate morpholino, or any combination thereof.

In certain embodiments, the DsiRNA comprises at least one 2′-O-methyl nucleotide. In certain embodiments, the DsiRNA comprises at least two 2′-O-methyl nucleotides. In certain embodiments, the DsiRNA comprises at least three 2′-O-methyl nucleotides. In certain embodiments, at least about 10, 20, 30, 40, 50, 60, 70, 80, 90 or 100% of the DsiRNA nucleotides are 2′-O-methyl modified.

In certain embodiments, the DsiRNA comprises at least one nucleotide with a phosphorothioate linkage. In certain embodiments, the DsiRNA comprises at least two nucleotides with phosphorothioate linkages. In certain embodiments, the DsiRNA comprises at least three nucleotides with phosphorothioate linkages. In certain embodiments, at least about 10, 20, 30, 40, 50, 60, 70, 80, 90 or 100% of the DsiRNA nucleotides comprise phosphorothioate linkages.

In certain embodiments, the DsiRNA comprises at least one phosphorothioate 2′-O-methyl modified nucleotide. In certain embodiments, the DsiRNA comprises at least two phosphorothioate 2′-O-methyl modified nucleotides. In certain embodiments, the DsiRNA comprises at least three phosphorothioate 2′-O-methyl modified nucleotides. In certain embodiments, at least about 10, 20, 30, 40, 50, 60, 70, 80, 90 or 100% of the DsiRNA nucleotides are phosphorothioate 2′-O-methyl modified.

In certain embodiments, modifications include a bicyclic sugar moiety similar to the LNA has also been described (see U.S. Pat. No. 6,043,060) where the bridge is a single methylene group which connect the 3′-hydroxyl group to the 4′ carbon atom of the sugar ring thereby forming a 3′-C,4′-C-oxymethylene linkage. In certain embodiments oligonucleotide modifications include cyclohexene nucleic acids (CeNA), in which the furanose ring of a DNA or RNA molecule is replaced with a cyclohexenyl ring to increase stability of the resulting complexes with RNA and DNA complements (Wang et al., J. Am. Chem. Soc., 2000, 122, 8595-8602). In certain embodiments other bicyclic and tricyclic nucleoside analogs are included in the DsiRNA.

The target RNA of the invention is highly sequence specific. In general, oligonucleotides containing nucleotide sequences perfectly complementary to a portion of the target RNA are preferred for blocking of the target RNA. However, 100% sequence complementarity between the oligonucleotide and the target RNA is not required to practice the present invention. Thus, the invention may tolerate sequence variations that might be expected due to genetic mutation, strain polymorphism, or evolutionary divergence. For example, oligonucleotide sequences with insertions, deletions, and single point mutations relative to the target sequence may also be effective for inhibition. Alternatively, oligonucleotide sequences with nucleotide analog substitutions or insertions can be effective for blocking. Greater than 70% sequence identity (or complementarity), e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or even 100% sequence identity, and any and all whole or partial increments there between the oligonucleotide and the target RNA.

In certain embodiments, “sequence identity” or “identity” in the context of two nucleic acid sequences makes reference to a specified percentage of residues in the two sequences that are the same when aligned by sequence comparison algorithms or by visual inspection. For example, sequence identity may be used to reference a specified percentage of residues that are the same across the entirety of the two sequences when aligned.

In certain embodiments, the term “substantial identity” of polynucleotide sequences means that a polynucleotide comprises a sequence that has at least 70%, 71%, 72%, 73%, 74%, 75%, 76%, 77%, 78%, or 79%; at least 80%, 81%, 82%, 83%, 84%, 85%, 86%, 87%, 88%, or 89%; at least 90%, 91%, 92%, 93%, or 94%; or even at least 95%, 96%, 97%, 98%, or 99% sequence identity, compared to a reference sequence using one of the alignment programs described using standard parameters.

Sequence identity, including determination of sequence complementarity for nucleic acid sequences, may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., % homology=number of identical positions/total number of positions×100), optionally penalizing the score for the number of gaps introduced and/or length of gaps introduced. The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A preferred, non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10.

In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A preferred, non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM 120 weight residue table, a gap length penalty of 12, and a gap penalty of 4 can be used.

In another embodiment, the sequence identity for two sequences is based on the number of consecutive identical nucleotides between the two sequences (without inserting gaps). For example, the percent sequence identity between Sequence A and B below would be 87.5% (Sequence B is 14/16 identical to Sequence A), whereas the percent sequence identity between Sequence A and C would be 37.5% (Sequence C is 6/16 identical to Sequence A).

Sequence A:
GCATGCATGCATGCAT
Sequence B:
GCATGCATGCATGC
Sequence C:
GCATTTGCAGCAGC

Alternatively, the oligonucleotide may be defined functionally as a nucleotide sequence (or oligonucleotide sequence) a portion of which is capable of hybridizing with the target RNA (e.g., 400 mM NaCl, 40 mM PIPES pH 6.4, 1 mM EDTA, 50° C. or 70° C. hybridization for 12-16 hours; followed by washing). Additional preferred hybridization conditions include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in IX SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(number of A+T bases)+4(number of G+C bases). For hybrids between 18 and 49 base pairs in length, Tm(° C.)=81.5+16.6(log 10[Na⁺])+0.41(% G+C)−(600/N), where N is the number of bases in the hybrid, and [Na⁺] is the concentration of sodium ions in the hybridization buffer ([Na⁺] for 1×SSC=0.165 M). Additional examples of stringency conditions for polynucleotide hybridization are provided in Sambrook, et al., 2001, Molecular Cloning: A Laboratory Manual, Cold Spring Harbor Laboratory Press, Cold Spring Harbor, N. Y., chapters 9 and 11, and Current Protocols in Molecular Biology, 1995, F. M. Ausubel et al., eds., John Wiley & Sons, Inc., sections 2.10 and 6.3-6.4, incorporated herein by reference. The length of the identical nucleotide sequences may be at least about 10, 12, 15, 17, 20, 22, 25, 27, 30, 32, 35, 37, 40, 42, 45, 47 or 50 bases.

Administration of Therapeutic Agent

The therapeutic agent is administered to the patient so that the therapeutic agent contacts cells of the patient's respiratory or digestive system. For example, the therapeutic agent may be administered directly via an airway to cells of the patient's respiratory system. The therapeutic agent can be administered intranasally (e.g., nose drops) or by inhalation via the respiratory system, such as by propellant based metered dose inhalers or dry powders inhalation devices.

Formulations suitable for administration include liquid solutions. Liquid formulations may include diluents, such as water and alcohols, for example, ethanol, benzyl alcohol, propylene glycol, glycerin, and the polyethylene alcohols, either with or without the addition of a pharmaceutically acceptable surfactant, suspending agent, or emulsifying agent. The therapeutic agent can be administered in a physiologically acceptable diluent in a pharmaceutically acceptable carrier, such as a sterile liquid or mixture of liquids, including water, saline, aqueous dextrose and related sugar solutions, an alcohol, such as ethanol, isopropanol, or hexadecyl alcohol, glycols, such as propylene glycol or polyethylene glycol such as poly(ethyleneglycol) 400, glycerol ketals, such as 2,2-dimethyl-1,3-dioxolane-4-methanol, ethers, an oil, a fatty acid, a fatty acid ester or glyceride, or an acetylated fatty acid glyceride with or without the addition of a pharmaceutically acceptable surfactant, such as a soap or a detergent, suspending agent, such as pectin, carbomers, methylcellulose, hydroxypropylmethylcellulose, or carboxymethylcellulose, or emulsifying agents and other pharmaceutical adjuvants.

The therapeutic agent, alone or in combination with other suitable components, can be made into aerosol formulations to be administered via inhalation. These aerosol formulations can be placed into pressurized acceptable propellants, such as dichlorodifluoromethane, propane, and nitrogen. Such aerosol formulations may be administered by metered dose inhalers. They also may be formulated as pharmaceuticals for non-pressured preparations, such as in a nebulizer or an atomizer. In certain embodiments, administration may be, e.g., aerosol, instillation, intratracheal, intrabronchial or bronchoscopic deposition.

In certain embodiments, the therapeutic agent may be administered in a pharmaceutical composition. Such pharmaceutical compositions may also comprise a pharmaceutically acceptable carrier and other ingredients known in the art. The pharmaceutically acceptable carriers described herein, including, but not limited to, vehicles, adjuvants, excipients, or diluents, are well-known to those who are skilled in the art. Typically, the pharmaceutically acceptable carrier is chemically inert to the active compounds and has no detrimental side effects or toxicity under the conditions of use. The pharmaceutically acceptable carriers can include polymers and polymer matrices. Viscoelastic gel formulations with, e.g., methylcellulose and/or carboxymethylcellulose may be beneficial (see Sinn et al., Am J Respir Cell Mol Biol, 32(5), 404-410 (2005)).

The therapeutic agent can be administered by any conventional method available for use in conjunction with pharmaceuticals, either as individual therapeutic agents or in combination with at least one additional therapeutic agent.

In certain embodiments, the therapeutic agent are administered with an agent that disrupts, e.g., transiently disrupts, tight junctions, such as EGTA (see U.S. Pat. No. 6,855,549).

The total amount of the therapeutic agent administered will also be determined by the route, timing and frequency of administration as well as the existence, nature, and extent of any adverse side effects that might accompany the administration of the compound and the desired physiological effect. It will be appreciated by one skilled in the art that various conditions or disease states, in particular chronic conditions or disease states, may require prolonged treatment involving multiple administrations.

The therapeutic agent can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The therapeutic agent may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compounds of formula I to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the therapeutic agent can be determined by comparing their in vitro activity, and in vivo activity in animal models. Methods for the extrapolation of effective dosages in mice, and other animals, to humans are known to the art; for example, see U.S. Pat. No. 4,938,949.

The amount of the therapeutic agent, or an active salt or derivative thereof, required for use in treatment will vary not only with the particular salt selected but also with the route of administration, the nature of the condition being treated and the age and condition of the patient and will be ultimately at the discretion of the attendant physician or clinician.

Pharmaceutical compositions are administered in an amount, and with a frequency, that is effective to inhibit or alleviate the symptoms of cystic fibrosis and/or to delay the progression of the disease. The effect of a treatment may be clinically determined by nasal potential difference measurements as described herein. The precise dosage and duration of treatment may be determined empirically using known testing protocols or by testing the compositions in model systems known in the art and extrapolating therefrom. Dosages may also vary with the severity of the disease. A pharmaceutical composition is generally formulated and administered to exert a therapeutically useful effect while minimizing undesirable side effects. In general, an oral dose ranges from about 200 mg to about 1000 mg, which may be administered 1 to 3 times per day. Compositions administered as an aerosol are generally designed to provide a final concentration of about 10 to 50 μM at the airway surface, and may be administered 1 to 3 times per day. It will be apparent that, for any particular subject, specific dosage regimens may be adjusted over time according to the individual need. In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Compounds of the invention can also be administered in combination with other therapeutic agents, for example, other agents that are useful to treat cystic fibrosis. Examples of such agents include antibiotics. Accordingly, in one embodiment the invention also provides a composition comprising a therapeutic agent, or a pharmaceutically acceptable salt thereof, at least one other therapeutic agent, and a pharmaceutically acceptable diluent or carrier. The invention also provides a kit comprising a therapeutic agent, or a pharmaceutically acceptable salt thereof, at least one other therapeutic agent, packaging material, and instructions for administering the therapeutic agent or the pharmaceutically acceptable salt thereof and the other therapeutic agent or agents to an animal to treat cystic fibrosis.

A pharmaceutical composition may be prepared with carriers that protect active ingredients against rapid elimination from the body, such as time release formulations or coatings. Such carriers include controlled release formulations, such as, but not limited to, microencapsulated delivery systems, and biodegradable, biocompatible polymers, such as ethylene vinyl acetate, polyanhydrides, polyglycolic acid, polyorthoesters, polylactic acid and others known to those of ordinary skill in the art.

In certain embodiments, the therapeutic agent is directly administered as a pressurized aerosol or nebulized formulation to the patient's lungs via inhalation. Such formulations may contain any of a variety of known aerosol propellants useful for endopulmonary and/or intranasal inhalation administration. In addition, water may be present, with or without any of a variety of cosolvents, surfactants, stabilizers (e.g., antioxidants, chelating agents, inert gases and buffers). For compositions to be administered from multiple dose containers, antimicrobial agents are typically added. Such compositions are also generally filtered and sterilized, and may be lyophilized to provide enhanced stability and to improve solubility.

As noted above, a therapeutic agent may be administered to a mammal to stimulate chloride transport, and to treat cystic fibrosis. Patients that may benefit from administration of a therapeutic compound as described herein are those afflicted with cystic fibrosis. Such patients may be identified based on standard criteria that are well known in the art, including the presence of abnormally high salt concentrations in the sweat test, the presence of high nasal potentials, or the presence of a cystic fibrosis-associated mutation. Activation of chloride transport may also be beneficial in other diseases that show abnormally high mucus accumulation in the airways, such as asthma and chronic bronchitis. Similarly, intestinal constipation may benefit from activation of chloride transport by the therapeutic agents provided herein.

The term “therapeutically effective amount,” in reference to treating a disease state/condition, refers to an amount of a compound either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat,” “treating” and “treatment” as used herein include administering a compound prior to the onset of clinical symptoms of a disease state/condition so as to prevent any symptom, as well as administering a compound after the onset of clinical symptoms of a disease state/condition so as to reduce or eliminate any symptom, aspect or characteristic of the disease state/condition. Such treating need not be absolute to be useful.

### Example 1

### Mining a MicroRNA-Regulated Gene Network Identifies Candidate Genes Involved in ΔF508-CFTR Rescue

Cystic Fibrosis (CF) is the most common, lethal genetic disease among populations of Caucasian and northern European descent. CF is caused by mutations in the gene CF transmembrane conductance regulator (CFTR)¹, a phosphorylation and nucleotide gated anion channel expressed in epithelial cells lining the airways, sweat duct, intestine, pancreatic duct, bile canaliculi, and the reproductive tract ^{2,3}. The majority of CF-associated morbidity and mortality arises from progressive pulmonary infection and inflammation. Approximately 90% of people with CF have at least one mutant ΔF508-CFTR allele, making it the most common CFTR mutation ^{4,5}. The ΔF508 mutation results in CFTR protein misfolding, retention in the ER, and degradation via the ERAD pathway ⁶⁻⁸.

There is consensus that both wild type and ΔF508-CFTR assume similar conformations early on, but aberrant folding caused by the deletion marks the mutant protein for degradation ^{7,9}. Of note, both wild type and ΔF508-CFTR proteins fold inefficiently ¹⁰. Only a fraction of wild type CFTR protein is released from chaperone complexes to mature in the Golgi and traffic to the plasma membrane ^{10,11}. The remainder is rapidly degraded by the proteasome in an ubiquitin-dependent manner ^{10,11}. By contrast, chaperone complexes release less than 1% of ΔF508-CFTR primary polyproteins ^{12, 13}. The remainder is rapidly and efficiently degraded by the proteasome, also in an ubiquitin-dependent manner ^{10,11}. ΔF508-CFTR is a conditional, temperature-sensitive mutation. When mutant protein trafficks to the plasma membrane, as occurs with low temperature ⁸ or chemical chaperone treatment ¹⁴, it retains channel function although its residency time and open-state probability are reduced ⁸; this finding has motivated the search for interventions that can shift more ΔF508-CFTR towards the plasma membrane. ¹⁵⁻¹⁷.

In this study, we searched for ERAD and ubiquitin/proteasome pathway components that could be altered to rescue ΔF508-CFTR maturation and function. This work was motivated by our discovery that miR-138 and SIN3A gene network influenced ΔF508-CFTR abundance, maturation, and anion channel function ¹⁸. Of note, transfection with a miR-138 mimic or a Dicer-substrate siRNA (DsiRNA) against SIN3A, concomitantly increased ΔF508-CFTR expression and Cl⁻ transport in primary CF airway epithelia, suggesting that these interventions act through other genes to re-direct ΔF508-CFTR from the ERAD pathway to the cell surface ¹⁸. Here we identify SYVN1 (Hrd1, E3 ubiquitin ligase), NEDD8 (neddylation), and FBXO2 (Fbs1, E3 ubiquitin ligase) as components of this gene network that controls ΔF508-CFTR trafficking to the cell surface. RNAi-mediated depletion of each of these factors increased ΔF508-CFTR protein maturation and significantly improved ΔF508-CFTR mediated anion transport. We propose a role for SYVN1 and FBXO2 as components of ER quality control (ERQC) complexes that degrade ΔF508-CFTR, and a new role for NEDD8 in regulating ΔF508-CFTR ubiquitination.

Materials and Methods

Primary Human Airway Epithelia:

Airway epithelia from human trachea and primary bronchus removed from organs donated for research were cultured at the air-liquid interface (ALI) (Karp, P. H. et al. An in vitro model of differentiated human airway epithelia. Methods for establishing primary cultures. Methods in molecular biology 188, 115-137 (2002)). These studies were approved by the Institutional Review Board of the University of Iowa. Briefly, airway epithelial cells were dissociated from native tissue by pronase enzyme digestion. Permeable membrane inserts (0.6 cm² Millipore-PCF, 0.33 cm² Costar-Polyester) pre-coated with human placental collagen (IV, Sigma) were seeded with freshly dissociated epithelia. Seeding culture media used was DMEM/F-12 medium supplemented with 5% FBS, 50 units/mL penicillin, 50 μg/mL streptomycin, 50 μg/mL gentamicin, 2 μg/mL fluconazole, and 1.25 μg/mL amphotericin B. For epithelia from cystic fibrosis (CF) patients, the following additional antibiotics were used for the first 5 days: 77 μg/mL ceftazidime, 12.5 μg/mL imipenem and cilastatin, 80 μg/mL tobramycin, 25 μg/mL piperacillin and tazobactam. After seeding, the cultures were maintained in DMEM/F-12 medium supplemented with 2% Ultroser G (USG, Pall Biosepra) and the above listed antibiotics.

RNA Isolation:

Total RNA from primary airway epithelial cells (human and pig), HeLa cells, CFBE cells was isolated using the mirVana™ miRNA isolation kit, TRIzol® Reagent (Life Technologies, Carlsbad, Calif.) (Ramachandran, S., Clarke, L. A., Scheetz, T. E., Amaral, M. D. & McCray, P. B., Jr. Microarray mRNA expression profiling to study cystic fibrosis. Methods Mol Biol 742, 193-212 (2011)), or the SV96 Total RNA Isolation System (Promega, Madison, Wis.), according to the manufacturer's protocol. Total RNA was tested for quality on an Agilent Model 2100 Bioanalyzer (Agilent Technologies, Santa Clara, Calif.). Only samples with an RNA integrity number (RIN) over 7.0 were selected for downstream processing.

Oligonucleotide Transfections:

These protocols were described in detail in Ramachandran, S. et al. Efficient delivery of RNA interference oligonucleotides to polarized airway epithelia in vitro. Am J Physiol Lung Cell Mol Physiol 305, L23-32 (2013). Briefly, freshly dissociated human airway epithelial cells, CFBE cells or HeLa cells were transfected in pre-coated 96 well plates (Costar) or Transwell™ Permeable Supports (0.33 cm² 0.4 μm polyester membrane, Costar 3470). Lipofectamine™ RNAiMAX (Invitrogen) was used as a reverse transfection reagent. Pre-coated (with human placental collagen Type IV, Sigma) substrates were incubated with the transfection mix comprising of Opti-MEM (Invitrogen), oligonucleotide (Integrated DNA Technologies) and Lipofectamine™ RNAiMAX (Invitrogen). 15-20 minutes later, 150,000 freshly dissociated cells suspended in DMEM/F-12 were added to each well/insert. Between 4-6 hrs later, all media from the apical surface was aspirated and complete media added to the basolateral surface. Media on the basolateral surface were changed every 3-4 days. For human primary epithelial cultures, USG media described above was used. For cultures from immortalized cell lines: HeLa, CFBE41o-(termed CFBE throughout) (Kunzelmann et al. Am. J. Respir. Cell Mol. Biol. 8, 522 (May, 1993)), complete media specific to each cell line was used (HeLa: MEM (Gibco)+10% FBS (Atlanta Biologicals)+1% Pen Strep (Gibco); CFBE: Advanced DMEM (Gibco)+1% L-Glutamine (Gibco)+10% FBS (Atlanta Biologicals)+1% Pen Strep (Gibco)).

Oligonucleotide Reagents:

Ten DsiRNAs were designed and screened against each gene (data not shown), and the two best performing DsiRNAs were taken forward for additional studies (Supplementary FIG. 2). The DsiRNAs were designed (Kim, D. H. et al. Synthetic dsRNA Dicer substrates enhance RNAi potency and efficacy. Nat Biotechnol 23, 222-226 (2005); Rose, S. D. et al. Functional polarity is introduced by Dicer processing of short substrate RNAs. Nucleic acids research 33, 4140-4156 (2005)), synthesized and validated (Behlke, M. A. Chemical modification of siRNAs for in vivo use. Oligonucleotides 18, 305-319 (2008); Collingwood, M. A. et al. Chemical modification patterns compatible with high potency dicer-substrate small interfering RNAs. Oligonucleotides 18, 187-200 (2008)) by Integrated DNA Technologies. All accompanying control sequences (Scr) were also generated by Integrated DNA Technologies.

| Seq Name | DsiRNA # | Antisense Strand Sequence | Sense Strand Sequence |
|---|---|---|---|
| DNAJB12 | 1 | /5Phos/rUrArCrArUrUrCrUrArCmUrUmUrCrGr | /5Phos/rArCrCrArUrGrGrCrArArCrGrAr |
| | | UrUrGrCrCmArUmGrGmUmUmU | ArArGrUrArGrArArUrGTA |
| DNAJB12 | 2 | /5Phos/rUrUrUrGrArCrArUrUrUmCrCmUrUrAr | /5Phos/rGrGrArArGrUrUrCrGrGrUrArAr |
| | | CrCrGrArAmCrUmUrCmCmUmU | GrGrArArArUrGrUrCrAAA |
| DERL1 | 1 | /5Phos/rUrGrCrArCrArGrUrUrGmGrUmArUrUr | /5Phos/rGrUrGrArArGrArArCrArArArUr |
| | | UrGrUrUrCmUrUmCrAmCmAmG | ArCrCrArArCrUrGrUrGCA |
| DERL1 | 2 | /5Phos/rUrUrCrArArCrCrUrUrAmArAmUrCrAr | /5Phos/rGrGrGrArArUrArArCrArUrGrAr |
| | | UrGrUrUrAmUrUmCrCmCmUmU | UrUrUrArArGrGrUrUrGAA |
| HSPA5 | 1 | /5Phos/rCrArArUrUrArCrArUrUmCrGmArGrAr | /5Phos/rArGrArArCrUrUrArArGrUrCrUr |
| | | CrUrUrArAmGrUmUrCmUmUmU | CrGrArArUrGrUrArArUTG |
| HSPA5 | 2 | /5Phos/rArGrArArGrCrUrUrCrUmCrAmCrArAr | /5Phos/rGrGrUrCrUrArArUrGrUrUrUrGr |
| | | ArCrArUrUmArGmArCmCmAmG | UrGrArGrArArGrCrUrUCT |
| HSPA8 | 1 | /5Phos/rUrGrUrCrArGrArArCrCmArUmArGrAr | /5Phos/rGrGrArGrGrUrGrUrCrUrUrCrUr |
| | | ArGrArCrAmCrCmUrCmCmUmC | ArUrGrGrUrUrCrUrGrACA |
| HSPA8 | 2 | /5Phos/rUrUrCrArGrUrUrCrUrUmCrAmArArUr | /5Phos/rCrCrCrGrUrGrCrCrCrGrArUrUr |
| | | CrGrGrGrCmArCmGrGmGmUmA | UrGrArArGrArArCrUrGAA |
| CANX | 1 | /5Phos/rArArUrCrArUrCrArArCmUrAmUrUrCr | /5Phos/rGrCrUrGrArUrCrGrArArGrArAr |
| | | UrUrCrGrAmUrCmArGmCmAmC | UrArGrUrUrGrArUrGrATT |
| CANX | 2 | /5Phos/rUrArUrCrArCrArArCrUmGrCmArArGr | /5Phos/rGrCrArGrUrArArArUrArCrUrUr |
| | | UrArUrUrUmArCmUrGmCmUmA | GrCrArGrUrUrGrUrGrATA |
| DAB2 | 1 | /5Phos/rGrUrUrGrCrArCrUrUrGmUrUmUrCrUr | /5Phos/rCrUrArArCrGrArArGrUrArGrAr |
| | | ArCrUrUrCmGrUmUrAmGmAmC | ArArCrArArGrUrGrCrAAC |
| DAB2 | 2 | /5Phos/rArCrCrCrGrArUrUrUrCmArGmUrUrUr | /5Phos/rCrCrArArArCrUrArArCrArArAr |
| | | GrUrUrArGmUrUmUrGmGmUmC | CrUrGrArArArUrCrGrGGT |
| SYVN1 | I | /5Phos/rGrUrGrGrGrCrCrArGrCmGrAmGrCrAr | /5Phos/rGrCrUrArUrGrArArCrUrUrGrCr |
| | | ArGrUrUrCmArUmArGmCmUmU | UrCrGrCrUrGrGrCrCrCAC |
| SYVN1 | 2 | /5Phos/rUrCrArUrCrUrGrArArAmCrUmGrUrCr | /5Phos/rArGrUrUrGrUrUrGrGrArGrArCr |
| | | UrCrCrArAmCrAmArCmUmCmU | ArGrUrUrUrCrArGrArUGA |
| HSPA1A | 1 | /5Phos/rUrGrArCrArArArCrArGmArAmArUrAr | /5Phos/rCrArUrUrUrCrCrUrArGrUrArUr |
| | | CrUrArGrGmArAmArUmGmCmA | UrUrCrUrGrUrUrUrGrUCA |
| HSPA1A | 2 | /5Phos/rCrArGrUrArUrArArArUmUrCmArUrCr | /5Phos/rUrArCrArUrGrCrArGrArGrArUr |
| | | UrCrUrGrCmArUmGrUmAmGmA | GrArArUrUrUrArUrArCTG |
| GRIP1 | 1 | /5Phos/rArGrCrArCrUrGrUrUrCmUrGmUrUrCr | /5Phos/rArGrArUrUrGrGrArGrUrGrArAr |
| | | ArCrUrCrCmArAmUrCmUmCmC | CrArGrArArCrArGrUrGCT |
| GRIP1 | 2 | /5Phos/rGrCrCrArCrGrUrUrGrAmUrUmGrArUr | /5Phos/rArGrCrArCrArGrArUrArArUrCr |
| | | UrArUrCrUmGrUmGrCmUmUmC | ArArUrCrArArCrGrUrGGC |
| MARCH2 | 1 | /5Phos/rCrArCrArGrGrArUrCrAmCrAmGrArCr | /5Phos/rGrCrArGrArGrCrCrUrArGrUrCr |
| | | UrArGrGrCmUrCmUrGmCmAmU | UrGrUrGrArUrCrCrUrGTG |
| MARCH2 | 2 | /5Phos/rUrCrUrCrCrArGrArCrAmGrCmUrCrUr | /5Phos/rGrCrCrGrUrGrCrArUrArArGrAr |
| | | UrArUrGrCmArCmGrGmCmAmC | GrCrUrGrUrCrUrGrGrAGA |
| HSPB1 | 1 | /5Phos/rUrUrGrGrUrCrUrUrGrAmCrCmGrUrCr | /5Phos/rCrGrGrArCrGrArGrCrUrGrArCr |
| | | ArGrCrUrCmGrUmCrCmGmGmG | GrGrUrCrArArGrArCrCAA |
| HSPB1 | 2 | /5Phos/rArGrCrGrUrGrUrArUrUmUrCmCrGrCr | /5Phos/rGrGrUrGrCrUrUrCrArCrGrCrGr |
| | | GrUrGrArAmGrCmArCmCmGmG | GrArArArUrArCrArCrGCT |
| CAPNS1 | 1 | /5Phos/rCrArGrUrGrUrCrGrArAmCrUmGrUrUr | /5Phos/rGrCrCrArUrArUrArCrArArArCr |
| | | UrGrUrArUmArUmGrGmCmCmU | ArGrUrUrCrGrArCrArCTG |
| CAPNS1 | 2 | /5Phos/rArUrGrUrUrArUrArGrAmGrAmUrGrCr | /5Phos/rArCrCrUrGrArArUrGrArGrCrAr |
| | | UrCrArUrUmCrAmGrGmUmGmG | UrCrUrCrUrArUrArArCAT |
| HSPA9 | 1 | /5Phos/rUrUrUrCrArArUrArUrCmArUmCrUrUr | /5Phos/rGrGrArUrUrArArGrCrArArArGr |
| | | UrGrCrUrUmArAmUrCmCmAmC | ArUrGrArUrArUrUrGrAAA |
| HSPA9 | 2 | /5Phos/rArGrGrUrArArUrUrGrGmUrCmCrUrUr | /5Phos/rGrGrArArGrArArUrUrCrArArGr |
| | | GrArArUrUmCrUmUrCmCmAmU | GrArCrCrArArUrUrArCCT |
| DNAJC3 | 1 | /5Phos/rUrCrArUrArCrArUrUrUmCrCmUrCrUr | /5Phos/rCrCrUrArUrUrUrGrArUrArGrAr |
| | | ArUrCrArAmArUmArGmGmCmC | GrGrArArArUrGrUrArUGA |
| DNAJC3 | 2 | /5Phos/rArUrArArUrCrUrUrUrGmUrGmCrUrUr | /5Phos/rGrGrUrCrUrArGrArGrArArArGr |
| | | UrCrUrCrUmArGmArCmCmUmU | CrArCrArArArGrArUrUAT |
| ATP6V1A | 1 | /5Phos/rArArArUrCrCrArUrArAmUrGmUrUrAr | /5Phos/rGrCrArGrGrUrArArArCrUrArAr |
| | | GrUrUrUrAmCrCmUrGmCmUmG | CrArUrUrArUrGrGrArUTT |
| ATP6V1A | 2 | /5Phos/rArArArUrGrCrUrUrArCmGrUmUrGrAr | /5Phos/rArGrArArArCrUrArGrCrUrCrAr |
| | | GrCrUrArGmUrUmUrCmUmUmA | ArCrGrUrArArGrCrArUTT |
| PPP2R1B | 1 | /5Phos/rArArUrGrGrGrCrArArGmUrUmCrUrCr | /5Phos/rArGrArArCrUrUrGrGrUrGrArGr |
| | | ArCrCrArAmGrUmUrCmUmUmU | ArArCrUrUrGrCrCrCrATT |
| PPP2R1B | 2 | /5Phos/rUrCrCrCrUrGrUrArArAmGrCmArUrUr | /5Phos/rUrCrUrArGrArUrArCrCrArArUr |
| | | GrGrUrArUmCrUmArGmAmAmU | GrCrUrUrUrArCrArGrGGA |
| RCN1 | 1 | /5Phos/rUrUrUrCrCrCrUrArCrCmUrCmUrArAr | /5Phos/rArGrArArArGrGrArArUrUrUrAr |
| | | ArUrUrCrCmUrUmUrCmUmUmU | GrArGrGrUrArGrGrGrAAA |
| RCN1 | 2 | /5Phos/rUrArUrUrCrArCrUrArUmUrUmCrArAr | /5Phos/rGrGrCrCrUrGrArUrCrUrUrUrGr |
| | | ArGrArUrCmArGmGrCmCmUmA | ArArArUrArGrUrGrArATA |
| MARCH3 | 1 | /5Phos/rUrArCrArArArCrArUrCmArAmArCrAr | /5Phos/rArGrGrArGrArCrArGrUrUrGrUr |
| | | ArCrUrGrUmCrUmCrCmUmUmU | UrUrGrArUrGrUrUrUrGTA |
| MARCH3 | 2 | /5Phos/rUrUrArGrCrArArArUrAmUrCmArUrAr | /5Phos/rGrCrUrGrCrArArUrCrArUrArUr |
| | | UrGrArUrUmGrCmArGmCmAmU | GrArUrArUrUrUrGrCrUAA |
| BAG2 | 1 | /5Phos/rGrGrUrUrUrCrUrArArUmUrGmUrUrUr | /5Phos/rGrUrGrUrCrArGrUrArGrArArAr |
| | | CrUrArCrUmGrAmCrAmCmUmU | CrArArUrUrArGrArArACC |
| BAG2 | 2 | /5Phos/rUrUrCrArCrArGrUrGrGmUrAmArArUr | /5Phos/rArCrCrArCrCrUrArUrArArUrUr |
| | | UrArUrArGmGrUmGrGmUmUmU | UrArCrCrArCrUrGrUrGAA |
| BAG1 | 1 | /5Phos/rCrUrUrCrArUrArArArCmUrGmCrUrCr | /5Phos/rArGrCrCrArCrArArUrArGrArGr |
| | | UrArUrUrGmUrGmGrCmUmUmU | CrArGrUrUrUrArUrGrAAG |
| BAG1 | 2 | /5Phos/rArUrUrArArGrCrArUrAmArAmUrUrAr | /5Phos/rGrCrUrCrUrArGrUrCrArUrArAr |
| | | UrUrUrArUrGrCrUrUrAAT | UrGrArCrUmArGmArGmCmCmA |
| GOPC | 1 | /5Phos/rArUrGrCrArArUrArGrCmUrAmArUrUr | /5Phos/rGrGrUrArGrArCrCrArUrArArUr |
| | | ArUrGrGrUmCrUmArCmCmAmC | UrArGrCrUrArUrUrGrCAT |
| GOPC | 2 | /5Phos/rCrArCrUrUrArArUrArUmUrCmCrUrUr | /5Phos/rCrGrUrArCrArGrUrUrArArArGr |
| | | UrArArCrUmGrUmArCmGmUmC | GrArArUrArUrUrArArGTG |
| SLC9A3R1 | 1 | /5Phos/rArCrUrCrUrGrCrArUrUmUrCmUrUrGr | /5Phos/rArCrGrArGrUrUrCrUrUrCrArAr |
| | | ArArGrArAmCrUmCrGmUmCmA | GrArArArUrGrCrArGrAGT |
| SLC9A3R1 | 2 | /5Phos/rUrArArArGrUrCrArGrGmGrAmArGrAr | /5Phos/rArGrArArCrUrArUrGrUrUrCrUr |
| | | ArCrArUrAmGrUmUrCmUmCmU | UrCrCrCrUrGrArCrUrUTA |
| RCN2 | 1 | /5Phos/rArGrGrArArArGrArCrUmUrUmGrUrUr | /5Phos/rGrCrCrArUrArUrGrArCrArArCr |
| | | GrUrCrArUmArUmGrGmCmAmC | ArArArGrUrCrUrUrUrCCT |
| RCN2 | 2 | /5Phos/rUrGrUrCrArArArUrUrCmCrAmCrUrCr | /5Phos/rGrCrArUrUrArUrGrGrUrGrArGr |
| | | ArCrCrArUmArAmUrGmCmUmA | UrGrGrArArUrUrUrGrACA |
| HSP09B1 | 1 | /5Phos/rUrGrArArGrUrGrArCrAmArUmArArCr | /5Phos/rArGrCrArGrArUrArArGrGrUrUrAr |
| | | CrUrUrArUmCrUmGrCmUmAmC | UrUrGrUrCrArCrUrUCA |
| HSP09B1 | 2 | /5Phos/rArCrCrCrGrArUrUrUrCmArGmUrUrUr | /5Phos/rCrCrArArArCrUrArArCrArArArCr |
| | | GrUrUrArGmUrUmUrGmGmUmC | UrGrArArArUrCrGrGGT |
| RNF128 | 1 | /5Phos/rArCrUrArArUrArUrArCmCrAmArUrCr | /5Phos/rGrGrArCrUrUrArArUrUrGrArUrUr |
| | | ArArUrUrArnArGmUrCmCmAmU | GrGrUrArUrArUrUrAGT |
| RNF128 | 2 | /5Phos/rUrUrUrArArArUrArGrCmUrCmUrArUr | /5Phos/rCrCrArArArGrUrUrArArArUrArGr |
| | | UrUrArArCmUrUmUrGmGmUmG | ArGrCrUrArUrUrUrAAA |
| SIN3A | 1 | /5Phos/rGrGrUrArGrUrArUrCrUmGrAmArUrUr | /5Phos/rGrCrGrArUrArCrArUrGrArArUrUr |
| | | CrArUrGrUtnArUmCrGmCmUmC | CrArGrArUrArCrUrACC |
| SIN3A | 2 | /5Phos/rUrArGrGrArArUrUrCrAmGrCmUrUrGr | /5Phos/rArGrUrGrUrArGrArUrUrCrArArGr |
| | | ArArUrCrUmArCmArCmUmCmC | CrUrGrArArUrUrCrCTA |
| SYVN1 3′UTR | 1 | /5Phos/rGrUrGrGrGrCrCrArGrCmGrAmGrCrAr | /5Phos/rGrCrUrArUrGrArArCrUrUrGrCrUr |
| | | ArGrUrUrCmArUmArGmCmUmU | CrGrCrUrGrGrCrCrCAC |
| SYVN1 CDS | 1 | /5Phos/rGrUrGrArGrGrUrArCrUmGrGmUrUrGr | /5Phos/rUrGrCrUrGrCrArGrArUrCrArArCr |
| | | ArUrCrUrGmCrAmGrCmAmUmG | CrArGrUrArCrCrUCAC |
| NEDD8 | 1 | /5Phos/rCrGrUrCrUrUrCrArCrUmUrUmArArUr | /5Phos/rGrArArGrArUrGrCrUrArArUrUrAr |
| | | UrArGrCrAmUrCmUrUmCmUmU | ArArGrUrGrArArGrACG |
| NEDD8 | 2 | /5Phos/rGrUrCrArArUrCrUrCrAmArUmCrUrCr | /5Phos/rGrArCrCrGrGrArArArGrGrArGrAr |
| | | CrUrUrUrCmCrGmGrUmCmAmG | UrUrGrArGrArUrUrGAC |
| NEDD8 | 3 | /5Phos/rUrCrCrCrUrCrUrUrUrCmUrCmCrUrCr | /5Phos/rGrGrArGrCrGrUrGrUrGrGrArGrGr |
| | | CrArCrArCmGrCmUrCmCmUmU | ArGrArArArGrArGrGGA |
| FBXO2 | 1 | /5Phos/rGrGrArCrGrCrUrArUrGmGrAmCrUrAr | /5Phos/rGrGrCrCrUrUrArArCrUrUrArGrUr |
| | | ArGrUrUrAmArGmGrCmCmUmA | CrCrArUrArGrCrGrUCC |
| FBXO2 | 2 | /5Phos/rUrCrArCrGrCrCrCrUrCmArCmGrGrAr | /5Phos/ArGrArArUrGrUrArGrArUrCrCrGrUr |
| | | UrCrUrArCmArUmUrCmUmAmG | GrArGrGrGrCrGrUGA |
| FBXO2 | 3 | /5Phos/rCrArCrGrUrUrCrUrCrGmUrGmCrUrCr | /5Phos/rGrCrUrArCrUrGrUrCrCrGrArGrCrAr |
| | | GrGrArCrAmGrUmArGmCmUmU | CrGrArGrArArCrGTG |
| AHSA1 | 1 | /5Phos/rCrCrArCrArUrGrUrCrCmUrUmUrGrUr | /5Phos/rGrGrArGrUrArCrArArUrArCrArArAr |
| | | ArUrUrGrUmArCmUrCmCmUmG | GrGrArCrArUrGrUGG |
| AMFR | 1 | /5Phos/rGrArArGrGrArUrUrArAmArUmUrUrAr | /5Phos/rGrGrArCrArGrCrUrGrArUrArArArUr |
| | | UrCrArGrCmUrGmUrCmCmAmA | UrUrArArUrCrCrUTC |
| RNF5 | 1 | /5Phos/rCrCrCrUrCrArArUrArCmUrGmArUrUr | /5Phos/rGrCrCrArGrArGrArArGrArArUrCrAr |
| | | CrUrUrCrUmCrUmGrGmCmUmG | GrUrArUrUrGrArGGG |

r = RNA
m = 2′OMe modification

Quantitative RT-PCR (RT-qPCR):

First-strand cDNA was synthesized using SuperScript® II (Invitrogen), and oligo-dT and random-hexamer primers. Sequence specific PrimeTime® qPCR Assays were optimized for the following human genes using protocols developed at Integrated DNA Technologies: SIN3A, DERL1, HSPA8, HSPA5, DNAJB12, BAG1, NHERF1, CAPNS1, HSPB1, HSPA1A, MARCH2, HSP90B1, RNF128, CANX, GRIP1, SYVN1, DAB2, RCN2, GOPC, HSPA9, MARCH3, PPP2RIB, RCN1, BAG2, STP6V1A, DNAJC3, CFTR, SIN3A, AMFR, RNF5, AHSA1, NEDD8, FBXO2, GAPDH, HPRT, and SFRS9. Quantitative RT PCR assays for porcine NEDD8 and GAPDH were also developed using Integrated DNA Technologies protocols. All reactions were setup using TaqMan® Fast Universal PCR Master Mix (Applied Biosystems) and run on the Applied Biosystems 7900 HT Real-Time PCR system. All experiments were performed in quadruplicate.

Electrophysiology Studies:

Transepithelial Cl⁻ current measurements were made in Ussing chambers at 2 weeks post-seeding (Itani, O. A. et al. Human cystic fibrosis airway epithelia have reduced Cl− conductance but not increased Na+ conductance. Proceedings of the National Academy of Sciences of the United States of America 108, 10260-10265 (2011)). Briefly, primary cultures or polarized air liquid interphase cultures were mounted in Ussing chambers (EasyMount P2300 chamber system, Physiologic Instruments, San Diego, Calif.) and voltage clamped (model VCCMC8-4S, Physiologic Instruments), and connected to a computerized data acquisition system (Acquire & Analyze 2.3.181, Physiologic Instruments) to record short-circuit currents and transepithelial resistance. Transepithelial Cl⁻ current was measured under short-circuit current conditions. After measuring baseline current, the transepithelial current (Iₜ) response to sequential apical addition of 100 μM amiloride (Amil), 100 μM 4,4′-diisothiocyanoto-stilbene-2,2′-disulfonic acid (DIDS), 4.8 mM [Cl⁻], 10 μM forskolin and 100 μM 3-isobutyl-1-methylxanthine (IBMX), and 100 μM GlyH-101 was measured. Studies were conducted with a Cl⁻ concentration gradient containing 135 mM NaCl, 1.2 mM MgCl₂, 1.2 mM CaCl₂, 2.4 mM K₂PO₄, 0.6 mM KH₂PO₄, 5 mM dextrose, and 5 mM Hepes (pH 7.4) on the basolateral surface, and gluconate substituted for Cl⁻ on the apical side.

SDS-PAGE and Immunoblotting:

Cell lines were washed with PBS and lysed in freshly prepared lysis buffer (1% Triton, 25 mM Tris pH 7.4, 150 mM NaCl, protease inhibitors (cOmplete™, mini, EDTA-free, Roche)) for 30 min at 4° C. The lysates were centrifuged at 14,000 rpm for 20 min at 4° C., and the supernatant quantified by BCA Protein Assay kit (Pierce). CFTR was denatured in 6×-Sample SDS buffer (375 mM Tris-HCl pH 6.8, 6% SDS, 48% glycerol, 9% 2-Mercaptoethanol, and 0.03% bromophenol blue). 20 μg (HeLa, CFBE) of protein per lane was separated on a 7% SDS-PAGE gel for western blot analysis. Protein abundance was quantified by densitometry using an AlphaInnotech Fluorochem Imager (AlphaInnotech). For CFTR, band B and C were quantified separately. Western blots were probed, stripped and re-probed as follows. PVDF membranes were first probed with the antibody against the gene of interest. After imaging, the PVDF membrane was stripped with Restore Western Blot Stripping Buffer (Thermo Scientific) for 15 minutes, washed in Tris Buffered Saline-Tween (TBS-T) and blocked in 5% Bovine Serum Albumin (BSA, Pierce) for 1 hr. The membrane was washed in TBS-T and incubated with the goat anti-mouse secondary antibody (1:10000, Sigma) for 1 hr and imaged. If signal was detected, the stripping procedure was repeated till no signal was observed. The membrane was washed in TBS-T, blocked for 1 hr in 5% BSA and re-probed with the antibody against tubulin.

| Protein | Antibody | Source |
|---|---|---|
| CFTR | R-769 | CFFT |
| Hemagglutinin | HA.11 Clone 16B12 Monoclonal Antibody | Covance |
| α-tubulin | clone DM1A | Sigma |
| SYVN1 | ab38456 | Abcam |
| NEDD8 | ab38634 | Abcam |
| FBXO2 | ab96391 | Abcam |
| AMFR | ab101284 | Abcam |
| RNF5 | ab128200 | Abcam |
| AHSA1 | ab56721 | Abcam |
| Ubiquitin | ab140601 | Abcam |

CFTR Ubiquitination Measurements:

Cells were treated with 10 μM MG-132 in the last 1-hour of incubation, and then lysed in lysis buffer (1% Triton, 25 mM Tris pH 7.4, 150 mM NaCl, protease inhibitors (cOmplete™, mini, EDTA-free, Roche), 5 mM N-ethylmaleimide (NEM) and 20 μM MG-132) for 30 min at 4° C. The lysates were centrifuged at 14,000 rpm for 20 min at 4° C., and the supernatant quantified by BCA Protein Assay kit (Pierce). CFTR was precipitated with the anti-HA antibody. The immunoprecipitates were analyzed by immunoblotting with anti-Ub and anti-HA antibodies. CFTR ubiquitination level with molecular masses >180 kDa was measured by densitometry and normalized for the CFTR level in the precipitate.

Immunoprecipitation:

Immunoprecipitation (IP) experiments were performed in HeLa cells stably expressing ΔF508-CFTR-HA. To IP ΔF508-CFTR, cells were lysed (as described above), and supernatant (20-50 μg of protein) was incubated with either anti-HA (to IP CFTR) or anti-AMFR for 1 h at 4° C., followed by incubation with protein G-agarose (Invitrogen) for 1 h at 4° C. Immunoprecipitates were washed 4 times with lysis buffer and eluted in 6× sample-SDS buffer. Samples were analyzed by immunoblotting as described above.

Measuring Cell Surface Display of CFTR:

Hela cells stably expressing wild-type CFTR or CFTR-ΔF508 were kindly provided by Dr. G. Lukacs (Sharma, M., Benharouga, M., Hu, W. & Lukacs, G. L. Conformational and temperature-sensitive stability defects of the delta F508 cystic fibrosis transmembrane conductance regulator in post-endoplasmic reticulum compartments. The Journal of biological chemistry 276, 8942-8950 (2001); Sharma, M. et al. Misfolding diverts CFTR from recycling to degradation: quality control at early endosomes. J Cell Biol 164, 923-933 (2004)). Cell surface ELISA was performed on these cells (Okiyoneda, T. et al. Peripheral protein quality control removes unfolded CFTR from the plasma membrane. Science 329, 805-810 (2010)) after noted treatments. HeLa cells were transfected/treated in 96 well plates (Costar). Briefly, the plate containing the cells was moved to a cold room (4° C.), and all media used was ice cold. Cells were washed with PBS, and blocked for 30 min with PBS containing 5% BSA. Anti-HA primary antibody (Covance) was added in 5% BSA-PBS at a 1:1000 concentration for 1 hr. Cells were washed with PBS, and anti-mouse secondary antibody HRP conjugated (Amersham) was added to cells at 1:1000 concentration in 5% BSA-PBS for 1 hr. Cells were washed thoroughly, and signal developed using SureBlue Reserve™ TMB Microwell Substrate (KPL). The reaction was stopped and read on a VersaMax™ Microplate Reader (Molecular Devices) at 540 nm using the SoftMax® Prof Software (Molecular Devices). For normalization, cells were lysed and total protein quantitated using the BCA Protein Assay kit (Pierce). The experiment was performed in quadruplicate, and the data presented as a mean±standard deviation of individual data points.

Pulse-Chase Live-Cells Surface ELISA:

The protocol is similar to that described for measuring surface display, except that each experiment was performed on 6 different 96 wells plates in identical fashion. All 6 plates containing the treated cells were moved to a cold room (4° C.), and all media used was ice cold. Cells were washed with PBS, and blocked for 30 min with PBS containing 5% BSA. Anti-HA primary antibody (Covance) was added in 5% BSA-PBS at a 1:1000 concentration for 1 hr. Cells were washed with PBS, and the plates representing time points 0.5, 1, 1.5, 2 and 4 hr were moved to a 37° C. incubator for the chase. Plate representing time point 0 was processed immediately. The rest of the protocol is identical to that described above.

LDH Cytotoxicity Assay:

Primary airway epithelial cultures from three non-CF human donors were transfected with the following reagents-siSCR, SYVN1 DsiRNA, NEDD8 DsiRNA, or untreated. The apical surface was washed, and the basolateral media collected on days 4, 8, 12, 16, 20, 28 and 28 post-transfection. LDH cytotoxicity assay kit (Cayman chemical) was used to measure the levels of lactate dehydrogenase in the washes and basolateral media. Percentage toxicity and viability were computed based on LDH levels. Data were normalized to untransfected cells and are presented in SI Fig. S6.

Histochemistry.

Epithelial sheets on filters were fixed with Zn formalin, embedded in paraffin, sectioned at 5 micron thickness, and stained with hematoxylin (Leica Biosystems) and eosin (Sigma) stain. Sections were visualized by light microscopy.

Statistical Analysis:

In all panels, error bars indicate standard error; statistical significance determined by the Holm-Bonferroni method; *P<0.05.

Results

RNAi Screen for Probing the miR-138/SIN3A Gene Network

The transcriptional changes in Calu-3 cells associated with the miR-138 mimic and SIN3A DsiRNA treatments were previously reported ¹⁸. Global mRNA transcript profiling identified a common set of 773 genes whose expression changed in response to these interventions. We identified a subset of 125 genes (Supplementary FIG. 1) that were co-regulated by the miR-138 mimic/SIN3A DsiRNA treatments and shared relational interactions with CFTR ¹⁸. We selected 25 candidate genes whose decreased expression correlated with elevated ΔF508 activity, suggesting possible involvement at early steps in CFTR biogenesis and transport based on known and predicted protein-protein interactions, protein cellular localization, and function with respect to CFTR biosynthesis.

The screening process used three metrics: (1) surface display of ΔF508-CFTR in HeLa cells stably expressing HA-tagged ΔF508-CFTR (HeLa-ΔF508-CFTR-HA), (2) improvement of ΔF508-CFTR maturation in CFBE cells demonstrated by the formation of fully glycosylated CFTR (band C), and (3) functional rescue of ΔF508-CFTR in CFBE cells measured as cAMP agonist induced transport. All assays were performed in parallel with two DsiRNAs individually (Supplementary FIG. 2, from this point onwards referred to as DsiRNAs) to reduce the possibility that observed rescue phenotypes were due to off-target effects.

RNAi Screen Reveals Role for SYVN1 in ΔF508-CFTR Biosynthesis

DsiRNAs targeting each gene were transfected into HeLa-ΔF508-CFTR-HA cells. 24 hrs post-transfection, ΔF508-CFTR surface display was measured using an anti-HA antibody. As negative controls, we transfected cells with a scrambled (siScr) oligonucleotide, or untreated cells (No Treatment, NoT). As positive controls, we reduced SIN3A expression with a DsiRNA or treated cells with the corrector compound C18 for 24 hrs (6 μM). We sought to identify genes whose knockdown restored ΔF508-CFTR trafficking to similar or higher levels of that achieved by either positive control. The DsiRNA-mediated inhibition of 12 genes restored trafficking significantly greater than the siScr transfected cells (FIG. 1A). Knockdown of SYVN1 (indicated with arrow) improved trafficking significantly greater than SIN3A inhibition or C18 treatment (FIG. 1A). Using DsiRNA in CFBE cells we also observed significant improvement in ΔF508-CFTR maturation by immunoblot (visualized as appearance of band C) with the knockdown of DERL1, HSPA8, HSPA5, BAG1, CAPNS1, HSPB1, HSP90B1, SYVN1, RNF128, RCN2, and BAG2 (FIG. 1B). CFBE cells were transfected with the same DsiRNAs, grown at the air-liquid interface (ALI)¹⁹, and mounted in Ussing chambers to measure CFTR Cl⁻ channel activity 4 days post-seeding. Consistent with surface display and band C appearance, knockdown of SYVN1 gave the greatest restoration of ΔF508-CFTR mediated transport in response to cAMP agonists (F & I), significantly more than C18 treatment alone (FIG. 1C).

NEDD8 Expression is Increased in CF Airways

In parallel to the screening of the 25 gene candidates, we identified an additional gene candidate while profiling for changes in mRNA expression between newborn CF and non-CF pig airways ²⁰. We observed significantly increased expression of NEDD8 (neural precursor cell expressed, developmentally downregulated 8) in CF airway epithelia. This observation was confirmed in additional human and pig well-differentiated primary airway epithelial cell cultures by RT-qPCR (Supplementary FIG. 3). Since NEDD8 is involved in regulating ubiquitination ²¹, we hypothesized that NEDD8 expression influences ΔF508-CFTR ubiquitination, and that NEDD8 inhibition will restore ΔF508-CFTR maturation in CF cells. Two separate DsiRNAs against NEDD8 (Supplementary FIG. 2) were used to inhibit its expression in HeLa-ΔF508-CFTR-HA cells and in CFBE cells. Loss of NEDD8 expression significantly improved ΔF508-CFTR surface display in HeLa cells (FIG. 1D), and improved ΔF508-CFTR maturation (FIG. 1E) and transport (FIG. 1F) in CFBE cells. The rescue phenotype observed with NEDD8 inhibition was significantly greater than that seen with SYVN1 knockdown, SIN3A knockdown, or C18 treatment (note differences in Y-axis scales). Based on these results, we focused additional studies on NEDD8 and SYVN1.

Loss of SYVN1 and NEDD8 Expression Reduces ΔF508-CFTR Ubiquitination

To elucidate the impact of SYVN1 and NEDD8 knockdown on CFTR, we measured its membrane stability by pulse-chase live-cell surface ELISA in HeLa-ΔF508-CFTR-HA cells. 24 hrs after transfecting cells with the reagents noted, we determined the ΔF508-CFTR membrane residence time at 5 time points after beginning the chase (chase performed at, 37° C.). While SYVN1 or NEDD8 knockdown increased ΔF508-CFTR trafficking to the membrane (FIG. 2A), pulse-chase experiments revealed that depletion of SYVN1 or NEDD8 did not extend the overall half-life of ΔF508-CFTR compared to the negative control (27° C. treatment) (FIG. 2B).

Since SYVN1 is an E3 ubiquitin ligase and NEDD8 plays a role in regulating ubiquitination ²¹, we next determined the impact of depleting SYVN1 and NEDD8 on ΔF508-CFTR ubiquitination. We transfected/treated HeLa-ΔF508-CFTR-HA cells with the reagents noted; 72 hrs later, we inhibited the proteasome with MG-132 (10 μM) for an hour, harvested protein, immunoprecipitated CFTR with an anti-HA antibody, and blotted for ubiquitin using an anti-ubiquitin antibody. SYVN1 and NEDD8 knockdown significantly reduced ΔF508-CFTR ubiquitination compared to the siScr control (FIG. 2C).

SYVN1/NEDD8 Knockdown Enhances ΔF508-CFTR Biosynthesis by Proteasome Inhibition

Reduced ΔF508-CFTR ubiquitination in response to inhibiting SYVN1 or NEDD8 expression suggests inactivation of the ΔF508-CFTR ubiquitination machinery or the chaperone complexes that target the misfolded protein for ubiquitination. Either of these scenarios might explain the observed partial restoration of ΔF508-CFTR trafficking, maturation, and function. We hypothesized that inhibiting SYVN1 or NEDD8 expression in concert with C18 (6 μM for 24 hrs) or low temperature (27° C. for 24 hrs) would enhance functional rescue of ΔF508-CFTR. We selected C18 and low temperature because, first, C18 is a class I corrector that interacts specifically with CFTR and has little impact on the ERQC/ubiquitination pathway ²², and second, ΔF508-CFTR processing is temperature sensitive ^{8, 22-24} and the effect of low temperature on expression levels of chaperones/co-chaperones in the ERQC/ubiquitination pathway is well characterized ²⁵⁻²⁷.

Combining SYVN1 or NEDD8 knockdown with C18 significantly increased ΔF508-CFTR trafficking to the membrane (FIG. 2A), increased ΔF508-CFTR membrane stability as measured by residence time (FIG. 2B), and increased maturation as measured by band C formation (FIG. 2D), compared to either treatment alone. The increased expression and stability at the plasma membrane suggests enhanced export of a more stable ΔF508-CFTR from the ER. However, the SYVN1 or NEDD8 knockdown induced reduction in ΔF508-CFTR ubiquitination was unaffected by combining the treatments with C18 (FIG. 2C), possibly because C18 treatment had little impact on ΔF508-CFTR ubiquitination levels.

On combining SYVN1 or NEDD8 knockdown with low temperature we again observed significantly increased trafficking of ΔF508-CFTR to the membrane (FIG. 2A), increased ΔF508-CFTR membrane stability as measured by residence time (FIG. 2B), and increased band C formation (FIG. 2D), compared to either treatment alone. We also observed a greater reduction in ΔF508-CFTR ubiquitination in comparison to low temperature or the SYVN1/NEDD8 knockdown treatments alone (FIG. 2C). Finally, significantly more ΔF508-CFTR Cl⁻ channel activity was observed in CFBE cells grown at air-liquid interface upon combining C18 or low temperature with SYVN1 or NEDD8 knockdown, compared to either treatments alone (FIG. 2E). Of note, combining SYVN1 or NEDD8 knockdown with low temperature restored ΔF508-CFTR trafficking, stability, and Cl⁻ transport to a lesser degree than that observed in combination with C18 (FIG. 2A, B, E).

SYVN1 Regulates ΔF508-CFTR Ubiquitination by the RNF5/AMFR Pathway

To understand how SYVN1 influences ΔF508-CFTR ubiquitination, we performed combinatorial RNAi knockdown of transcripts encoding proteins known to interact with ΔF508-CFTR in the ER. We hypothesized that a combinatorial gene silencing approach would help identify the pathways via which SYVN1 interacts and targets ΔF508-CFTR to the proteasome. We validated 2 different DsiRNA against the genes RNF5 (RMA1, ring finger protein 5, E3 ubiquitin protein ligase), AMFR (Gp78, autocrine motility factor receptor, E3 ubiquitin protein ligase), and AHSA1 (AHA1, activator of heat shock 90 kDa protein ATPase homolog 1 (yeast)) (Supplementary FIG. 4). RNF5 and AMFR are integral to the ΔF508-CFTR ubiquitination machinery in the ER ^{28, 29}. Owing to the role RNF5 plays as a quality control checkpoint in the ER ²⁸, we hypothesized that SYVN1 might regulate CFTR ubiquitination via the same checkpoint. We included AHSA1 as inhibition of this gene was reported to rescue ΔF508-CFTR maturation and trafficking ¹⁷. Of note, AHSA1 is proposed to stimulate Hsp90 ATPase activity, thereby regulating chaperone-mediated degradation of ΔF508-CFTR ¹⁷, a mechanism independent of the ER-based ubiquitination machinery. We also included DERL1 since it interacts with the ERAD machinery associated with ΔF508-CFTR degradation (FIGS. 1A-C). Knockdown of AMFR, RNF5, DERL1, or AHSA1 improved ΔF508-CFTR trafficking (data not shown) and maturation in CFBE cells (Supplementary FIG. 5) to varying degrees.

Using co-transfected DsiRNAs, we simultaneously reduced expression of SYVN1 together with either AMFR, RNF5, DERL1, or AHSA1. While SYVN1 knockdown increased ΔF508-CFTR trafficking in HeLa cells (FIG. 3A), maturation in CFBE cells (FIG. 3B), and function in ALI cultures of CFBE cells (FIG. 3C), combining SYVN1 knockdown with inhibition of AMFR, RNF5, or DERL1 failed to yield greater levels of rescue. Significantly greater rescue was observed only with the combined knockdown of SYVN1 and AHSA1 (FIGS. 3A-C). SYVN1 knockdown reduced ΔF508-CFTR ubiquitination (FIG. 3D), and only the dual inhibition of SYVN1 and AMFR yielded greater reduction in ΔF508-CFTR ubiquitination (FIG. 3D), perhaps owing to the role AMFR has in extending ΔF508-CFTR ubiquitin chains as an E4 ligase ³⁰.

These results suggest that SYVN1 is either part of, or regulates, the RNF5/AMFR ubiquitination machinery. To confirm this, we first transfected HeLa cells with either wild type SYVN1 (SYVN1exp) or a catalytically inactive SYVN1 (SYVN1mut) cDNA. Transfection of SYVN1exp cDNA did not alter surface display (FIG. 3E) or ΔF508-CFTR ubiquitination (FIG. 3F). However, expression of catalytically inactive SYVN1 improved ΔF508-CFTR trafficking (FIG. 3E), and reduced ΔF508-CFTR ubiquitination (FIG. 3F) to an extent similar to that seen with SYVN1 knockdown. Next, we performed complementation experiments, in which we inhibited SYVN1 expression with a DsiRNA, and also transfected either SYVN1exp or SYVN1mut using cDNA expression vectors. Addition of the SYVN1exp cDNA abrogated the rescue phenotype seen with SYVN1 knockdown (FIG. 3E, F). In contrast, expression of the catalytically inactive SYVN1 increased ΔF508-CFTR trafficking, and reduced ΔF508-CFTR ubiquitination significantly more than SYVN1 knockdown alone (FIG. 3E, F). These results indicate that the rescue phenotype observed with SYVN1 knockdown is due to the loss of its catalytic activity. We also found that overexpression of AMFR suppressed the ability of SYVN1 depletion to restore ΔF508-CFTR activity, while overexpression of catalytically inactive AMFR potentiated rescue (FIGS. 3E-F). These results suggest that AMFR acts downstream or in parallel with SYVN1 to ubiquitinate ΔF508-CFTR.

NEDD8 Regulates ΔF508-CFTR Ubiquitination Via the SCF^{FBXO2} Complex

FBXO2 (F-box protein-2, Fbs1/FBX2), an E3 ubiquitin ligase, was previously implicated in the ubiquitin-mediated degradation of ΔF508-CFTR via the SCF^{FBXO2} complex ³¹. As neddylation (NEDD8 attachment) is essential to activate the SCF complex, this directly links our results with NEDD8 knockdown and its effect on ΔF508-CFTR degradation. This suggests that the SCF^{FBXO2} complex is involved in the ubiquitination and degradation of ΔF508-CFTR in airway epithelia.

We validated 2 different DsiRNA against FBXO2 (Supplementary FIG. 4). Inhibition of FBXO2 expression improved ΔF508-CFTR trafficking in HeLa cells (FIG. 4A), maturation in CFBE cells (FIG. 4B, Supplementary FIG. 5), and function in ALI cultures of CFBE cells (FIG. 4C). FBXO2 knockdown also significantly reduced ΔF508-CFTR ubiquitination (FIG. 4D). These results indicate that FBXO2 is involved in the ΔF508-CFTR ubiquitination pathway.

We next used combinatorial gene knockdown experiments to assess the interactions between NEDD8 and FBXO2 with respect to ΔF508-CFTR ubiquitination and trafficking. We co-transfected DsiRNAs to reduce NEDD8 and FBXO2 expression either alone or in combination. Remarkably, NEDD8 knockdown, FBXO2 knockdown, or the combined knockdown of NEDD8+FBXO2 all yielded similar improvements in ΔF508-CFTR trafficking in HeLa cells (FIG. 4A), maturation in CFBE cells (FIG. 4B), function in ALI cultures of CFBE cells (FIG. 4C), and reduction in ΔF508-CFTR ubiquitination (FIG. 4D). However, combining SYVN1 knockdown with either NEDD8 or FBXO2 inhibition, further improved ΔF508-CFTR trafficking, maturation, function, and reduction in ubiquitination (FIGS. 4A-D). These results suggest that FBXO2 and NEDD8 may act via the same pathway.

Of note, the combined knockdown of NEDD8 and SYVN1 conferred the greatest improvement in ΔF508-CFTR biosynthesis. Trafficking, maturation, and functional rescue of the mutant protein was significantly higher, and greater than SYVN1 or NEDD8 knockdown alone (FIGS. 4A-C). ΔF508-CFTR ubiquitination was also greatly reduced by the combined knockdown of both gene products (FIG. 4D). These results suggest that SYVN1 and NEDD8, while acting via different pathways, are complementary in targeting mutant CFTR to the proteasome.

Inhibiting SYVN1 and NEDD8 Expression Rescues cAMP-Dependent Anion Transport in Primary CF Airway Epithelia

Encouraged by these results we next reduced SYVN1, NEDD8, and FBXO2 expression in primary CF airway epithelial cells. 14 days post-transfection, we observed significantly improved cAMP-activated Cl⁻ channel activity in primary cells obtained from a total of 7 human donors (FIG. 5A). Combining these individual treatments with C18 further improved CFTR-dependent anion transport (FIG. 5A).

To evaluate the possibility that the knockdown of either SYVN1 or NEDD8 is associated with cytotoxicity, we transfected primary airway epithelial cells from 3 non-CF donors and grew them at the ALI. We measured LDH release from the apical and basolateral compartments at 4 day intervals for 28 days (Supplementary FIG. 6), and performed hematoxylin and eosin (H&E) staining on similar cultures at days 14 and 28 to assess changes in cell morphology (Supplementary FIG. 7). No differences were observed between untreated (NoT), scrambled oligo transfected (siScr), SYVN1 DsiRNA, or the NEDD8 DsiRNA transfected cultures. These data suggest that prolonged inhibition of SYVN1 or NEDD8 expression is well tolerated by airway epithelial cells.

### DISCUSSION

Here we show that inhibiting SYVN1 expression partially restored processing and function of ΔF508-CFTR in primary CF airway epithelial cells (FIG. 5A). This rescue phenotype was in part due to the repression of the ERQC/ubiquitination machinery that targets ΔF508-CFTR to proteasomal degradation. We selected 125 candidate genes for a loss of function screen, focusing on protein products that might influence ΔF508-CFTR biosynthesis. This strategy allowed us to test the hypothesis that a subset of genes co-regulated by miR-138 and SIN3A recapitulated the previously reported rescue phenotype ¹⁸. SYVN1 emerged as the most promising candidate from the RNAi screen. Notably, inhibition of SYVN1 expression decreased ΔF508-CFTR ubiquitination. This result suggested that SYVN1, an E3 ubiquitin ligase, was involved in regulating ΔF508-CFTR polyubiquitination.

While SYVN1 inhibition increased ΔF508-CFTR membrane trafficking, it had little impact on its membrane stability. Following inhibition of proteosomal targeting, the folding defect persists, resulting in a protein with reduced membrane residence time and partial function. This finding was also observed upon combining SYVN1 knockdown with low temperature. While we observed increased surface display, membrane stability was unchanged, further indicating that the stability of the mutant protein is unchanged. We observed significantly less ubiquitinated ΔF508-CFTR on combining SYVN1 knockdown with low temperature. This result was not observed when combining SYVN1 knockdown with C18. Of note, combining SYVN1 knockdown with C18 significantly increased membrane stability, as C18 is a chemical chaperone that interacts directly with CFTR and partially rescues the folding defect. Furthermore, the effect of combining SYVN1 knockdown with C18 on ΔF508-CFTR function was higher than that seen with low temperature, a suggesting that low temperature and SYVN1 knockdown may share a group or groups of differentially regulated genes. If such an overlap exists it might provide insights into to how, in the presence of low temperature or SYVN1 knockdown, ΔF508-CFTR escapes the Hsc70/CHIP E3 complex that monitors the conformation of different regions of nascent CFTR ¹⁵.

Multiple pathways contribute to ΔF508-CFTR ubiquitination and delivery to the proteasome, a feature we exploited in determining if SYVN1 was part of the RNF5-AMFR network. We noticed that combining SYVN1 knockdown with inhibition of RNF5 or AMFR failed to further enhance the rescue phenotype observed with the SYVN1 DsiRNA treatment alone. However, increased rescue was observed on combining SYVN1 and AHSA1 knockdown, suggesting that SYVN1 either regulates RNF5-AMFR mediated ubiquitination of ΔF508-CFTR or is involved in the pathway. This conclusion is further supported by the observations that expression of a catalytically inactive SYVN1 recapitulated the rescue phenotype observed with SYVN1 knockdown, while the over-expression of wild-type AMFR abrogated it. Interestingly, the precise role of SYVN1 in ΔF508-CFTR degradation is controversial. Ballar et al reported that silencing or overexpressing SYVN1 decreased and increased ΔF508-CFTR levels, respectively ³². Our studies provide contrasting results. Here we studied native CFTR, SYVN1 and AMFR gene products in a relevant CF airway epithelial cell line, while Ballar and colleagues studied fusion proteins in heterologous cell systems. We also use modified DsiRNAs to inhibit gene expression, a system with high potency, high reproducibility, and a low off-target profile ³³. Moreover, AMFR is a highly unstable protein in contrast to the more stable SYVN1 ^{34, 35}, suggesting our was approach captured the dynamic interaction between AMFR and SYVN1, and their influence on CFTR. Our findings also corroborate those of Okiyoneda and coworkers who showed that SYVN1 inhibition improved ΔF508-CFTR trafficking to the plasma membrane ³⁶. Additionally, Gnann et al. demonstrated that SYVN1 knockdown in yeast stabilized ΔF508-CFTR ³², while Morito and coworkers showed that overexpression of native or RING finger mutant SYVN1 had no impact on ΔF508-CFTR ubiquitination ³⁰. These differences may reflect the model systems investigated.

NEDD8 knockdown resulted in partial rescue of ΔF508-CFTR processing and function and decreased ΔF508-CFTR ubiquitination (FIGS. 1, 2, 4, 5A). We selected NEDD8 because its transcript abundance was significantly increased in CF airway epithelia. NEDD8 stimulates ubiquitination via the cullin-RING ubiquitin ligase (CRL) complexes upon covalent attachment to cullin. CRLs constitute the largest group of E3 ubiquitin ligases, comprising >40% of all ubiquitin ligases ³⁸. Our data suggest a model wherein the positive effect of NEDD8 on ΔF508-CFTR rescue relates to its influence on the activity of the Cull-based E3 ligase complex, SCF^{FBXO2} (shown schematically in FIG. 5B). Importantly, the SCF^{FBXO2} complex binds specifically to proteins attached to N-linked high-mannose oligosaccharides and contributes to ubiquitination of N-glycosylated proteins ³¹. FBXO2 is an E3 ligase that directly interacts with ΔF508-CFTR; others include CHIP, RMA1, NEDD4-2, and AMFR (also an E4 ligase)²⁹. Yoshida and colleagues reported that ΔF508-CFTR is ubiquitinated by the SCF^{FBXO2} complex, and that loss of the F-box domain in FBXO2 significantly suppressed ΔF508-CFTR degradation ³¹. Their results suggest a possible mechanism for how inhibition of NEDD8 might reduce ΔF508-CFTR degradation. While it has been suggested that FBXO2 is expressed mainly in neuronal cells ³¹, we observed significant expression in respiratory epithelial cell lines including Calu-3, HBE, and CFBE cells, as well as in well-differentiated primary cultures of CF and non-CF airway epithelia (data not shown). Therefore, the SCF^{FBXO2} complex may contribute significantly to the ubiquitination and degradation of ΔF508-CFTR in airway epithelia.

Directly inhibiting FBXO2 expression also partially restored ΔF508-CFTR trafficking, maturation, and function; while simultaneously reducing ΔF508-CFTR ubiquitination. The failure of the combined knockdown of NEDD8 and FBXO2 to exhibit additive effects on ΔF508-CFTR rescue supports the notion that FBXO2 acts downstream of NEDD8. Further studies are needed to understand whether the SCF^{FBXO2} complex is the only NEDD8 regulated complex ubiquitinating ΔF508-CFTR.

In summary, inhibition of SYVN1 (Hrd1, E3 ubiquitin ligase), FBXO2 (Fbs1, E3 ubiquitin ligase), or NEDD8 (neddylation) partially rescued ΔF508-CFTR protein maturation and significantly improved ΔF508-CFTR mediated transport. These results suggest that SYVN1 and FBXO2 are components of ERQC complexes that degrade ΔF508-CFTR, and identify a new role for NEDD8 in regulating ΔF508-CFTR ubiquitination. Our findings provide new knowledge of the CFTR biosynthetic pathway and represent an important proof of principle for this discovery strategy. The gene products identified using this strategy may represent new targets for CF therapies.

### REFERENCES FOR EXAMPLE 1

1. Rowe, S. M., Miller, S. & Sorscher, E. J. Cystic fibrosis. N Engl J Med 352, 1992-2001 (2005).
2. Anderson, M. P. et al. Demonstration that CFTR is a chloride channel by alteration of its anion selectivity. Science 253, 202-205 (1991).
3. Anderson, M. P., Rich, D. P., Gregory, R. J., Smith, A. E. & Welsh, M. J. Generation of cAMP-activated chloride currents by expression of CFTR. Science 251, 679-682 (1991).
4. Kerem, B. et al. Identification of the cystic fibrosis gene: genetic analysis. Science 245, 1073-1080 (1989).
5. Tsui, L. C. Mutations and sequence variations detected in the cystic fibrosis transmembrane conductance regulator (CFTR) gene: a report from the Cystic Fibrosis Genetic Analysis Consortium. Hum Mutat 1, 197-203 (1992).
6. Cheng, S. H. et al. Defective intracellular transport and processing of CFTR is the molecular basis of most cystic fibrosis. Cell 63, 827-834 (1990).
7. Ward, C. L., Omura, S. & Kopito, R. R. Degradation of CFTR by the ubiquitin-proteasome pathway. Cell 83, 121-127 (1995).
8. Denning, G. M. et al. Processing of mutant cystic fibrosis transmembrane conductance regulator is temperature-sensitive. Nature 358, 761-764 (1992).
9. Zhang, F., Kartner, N. & Lukacs, G. L. Limited proteolysis as a probe for arrested conformational maturation of delta F508 CFTR. Nature Structural Biology 5, 180-183 (1998).
10. Ward, C. L. & Kopito, R. R. Intracellular turnover of cystic fibrosis transmembrane conductance regulator. Inefficient processing and rapid degradation of wild-type and mutant proteins. The Journal of Biological Chemistry 269, 25710-25718 (1994).
11. Lukacs, G. L. et al. Conformational maturation of CFTR but not its mutant counterpart (delta F508) occurs in the endoplasmic reticulum and requires ATP. The EMBO journal 13, 6076-6086 (1994).
12. Yang, Y. et al. Molecular basis of defective anion transport in L cells expressing recombinant forms of CFTR. Human Molecular Genetics 2, 1253-1261 (1993).
13. Pind, S., Riordan, J. R. & Williams, D. B. Participation of the endoplasmic reticulum chaperone calnexin (p88, IP90) in the biogenesis of the cystic fibrosis transmembrane conductance regulator. The Journal of Biological Chemistry 269, 12784-12788 (1994).
14. Brown, C. R., Hong-Brown, L. Q., Biwersi, J., Verkman, A. S. & Welch, W. J. Chemical chaperones correct the mutant phenotype of the delta F508 cystic fibrosis transmembrane conductance regulator protein. Cell Stress Chaperones 1, 117-125. (1996).
15. Meacham, G. C., Patterson, C., Zhang, W., Younger, J. M. & Cyr, D. M. The Hsc70 co-chaperone CHIP targets immature CFTR for proteasomal degradation. Nature Cell Biology 3, 100-105 (2001).
16. Okiyoneda, T., Apaja, P. M. & Lukacs, G. L. Protein quality control at the plasma membrane. Curr Opin Cell Biol 23, 483-491 (2011).
17. Wang, X. et al. Hsp90 cochaperone Aha1 downregulation rescues misfolding of CFTR in cystic fibrosis. Cell 127, 803-815 (2006).
18. Ramachandran, S. et al. A microRNA network regulates expression and biosynthesis of wild-type and DeltaF508 mutant cystic fibrosis transmembrane conductance regulator. Proceedings of the National Academy of Sciences of the United States of America 109, 13362-13367 (2012).
19. Ramachandran, S. et al. Efficient delivery of RNA interference oligonucleotides to polarized airway epithelia in vitro. Am J Physiol Lung Cell Mol Physiol 305, L23-32 (2013).
20. Stoltz, D. A. et al. Cystic fibrosis pigs develop lung disease and exhibit defective bacterial eradication at birth. Sci Transl Med 2, 29ra31 (2010).
21. Bosu, D. R. & Kipreos, E. T. Cullin-RING ubiquitin ligases: global regulation and activation cycles. Cell Division 3, 7 (2008).
22. Okiyoneda, T. et al. Mechanism-based corrector combination restores DeltaF508-CFTR folding and function. Nat Chem Biol 9, 444-454 (2013).
23. Wang, X., Koulov, A. V., Kellner, W. A., Riordan, J. R. & Balch, W. E. Chemical and biological folding contribute to temperature-sensitive DeltaF508 CFTR trafficking. Traffic 9, 1878-1893 (2008).
24. Sharma, M., Benharouga, M., Hu, W. & Lukacs, G. L. Conformational and temperature-sensitive stability defects of the delta F508 cystic fibrosis transmembrane conductance regulator in post-endoplasmic reticulum compartments. The Journal of Biological Chemistry 276, 8942-8950 (2001).
25. Zhang, D. et al. Ouabain Mimics Low Temperature Rescue of F508del-CFTR in Cystic Fibrosis Epithelial Cells. Frontiers in Pharmacology 3, 176 (2012).
26. Gomes-Alves, P., Neves, S., Coelho, A. V. & Penque, D. Low temperature restoring effect on F508del-CFTR misprocessing: A proteomic approach. J Proteomics 73, 218-230 (2009).
27. Sondo, E. et al. Rescue of the mutant CFTR chloride channel by pharmacological correctors and low temperature analyzed by gene expression profiling. Am J Physiol Cell Physiol 301, C872-885 (2011).
28. Younger, J. M. et al. Sequential quality-control checkpoints triage misfolded cystic fibrosis transmembrane conductance regulator. Cell 126, 571-582 (2006).
29. Lukacs, G. L. & Verkman, A. S. CFTR: folding, misfolding and correcting the DeltaF508 conformational defect. Trends Mol Med 18, 81-91 (2012).
30. Morito, D. et al. Gp78 cooperates with RMA1 in endoplasmic reticulum-associated degradation of CFTRDeltaF508. Mol Biol Cell 19, 1328-1336 (2008).
31. Yoshida, Y. et al. E3 ubiquitin ligase that recognizes sugar chains. Nature 418, 438-442 (2002).
32. Ballar, P., Ors, A. U., Yang, H. & Fang, S. Differential regulation of CFTRDeltaF508 degradation by ubiquitin ligases gp78 and Hrd1. Int J Biochem Cell Biol 42, 167-173 (2010).
33. Collingwood, M. A. et al. Chemical modification patterns compatible with high potency dicer-substrate small interfering RNAs. Oligonucleotides 18, 187-200 (2008).
34. Fang, S. et al. The tumor autocrine motility factor receptor, gp78, is a ubiquitin protein ligase implicated in degradation from the endoplasmic reticulum. Proc Natl Acad Sci USA 98, 14422-14427 (2001).
35. Kikkert, M. et al. Human HRD1 is an E3 ubiquitin ligase involved in degradation of proteins from the endoplasmic reticulum. J Biol Chem 279, 3525-3534 (2004).
36. Okiyoneda, T. et al. Peripheral protein quality control removes unfolded CFTR from the plasma membrane. Science 329, 805-810 (2010).
37. Gnann, A., Riordan, J. R. & Wolf, D. H. Cystic fibrosis transmembrane conductance regulator degradation depends on the lectins Htm1p/EDEM and the Cdc48 protein complex in yeast. Mol Biol Cell 15, 4125-4135 (2004).

38. Soucy, T. A. et al. An inhibitor of NEDD8-activating enzyme as a new approach to treat cancer. Nature 458, 732-736 (2009).

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of reducing ΔF508-CFTR ubiquitination or degradation, or increasing ΔF508-CFTR processing or function in a cystic fibrosis (CF) cell comprising contacting the cell with a therapeutic agent, wherein the therapeutic agent comprises one or more of
(a) a NEDD8 therapeutic agent that inhibits NEDD8 expression in the cell,
(b) a FBXO2 therapeutic agent that inhibits FBXO2 expression in the cell,
(c) a SYVN1 therapeutic agent that inhibits SYVN1 expression and a AHSA1 therapeutic agent that inhibits AHSA1 expression in the cell, or
(d) a therapeutic agent that inhibits SYVN1 expression in the cell.

2. The method of claim 1, wherein the therapeutic agent comprises
(a) the NEDD8 therapeutic agent and NEDD8 expression is inhibited by at least about 10%;
(b) the FBXO2 therapeutic agent that inhibits the F-box domain in FBXO2 and FBXO2 expression is inhibited by at least about 10%;
(c) the SYVN1 therapeutic agent that inhibits SYVN1 expression by at least about 10% and a AHSA1 therapeutic agent that inhibits AHSA1 expression by at least about 10%, or
(d) a therapeutic agent that inhibits SYVN1 expression by at least about 10%.

3. The method of claim 1, wherein the therapeutic agent is an siRNA oligonucleotide, an ASO oligonucleotide, a small molecule inhibitor, and/or other chemical inhibitor.

4. The method of claim 1, wherein the therapeutic agent comprises a NEDD8 therapeutic agent, and the NEDD8 therapeutic agent is
(a) an siRNA oligonucleotide having at least 90% identity to SEQ ID NO:6, SEQ ID NO:7, SEQ ID NO:8, SEQ ID NO:9, SEQ ID NO:10, or SEQ ID NO:11; and/or
(b) a small molecule inhibitor, and the small molecule inhibitor is MLN4924;
6,6″-biapigenin; and/or piperacillin.

5. The method of claim 1, wherein the therapeutic agent comprises a combination of a NEDD8 therapeutic agent and a FBXO2 therapeutic agent that inhibits FBXO2 expression in the cell.

6. The method of claim 3, wherein the therapeutic agent comprises a FBXO2 therapeutic agent that is an siRNA oligonucleotide having at least 90% identity to SEQ ID NO:12, SEQ ID NO:13, SEQ ID NO:14, SEQ ID NO:15, SEQ ID NO:16, or SEQ ID NO:17.

7. The method of claim 1, further comprising contacting the cell with a CFTR corrector and/or CFTR potentiator.

8. The method of claim 7, wherein the CFTR corrector is a small molecule CFTR corrector, a chemical chaperone and/or a proteostasis inhibitor.

9. The method of claim 7, wherein the CFTR corrector comprises one or more of the following:

| | Other | |
|---|---|---|
| Corrector | Name | Chemical Name |
| C1 | | 6-(1H-Benzoimidazol-2-ylsulfanylmethyl)-2-(6-methoxy- |
| | | 4-methyl-quinazolin-2-ylamino)-pyrimidin-4-ol |
| C2 | VRT-640 | 2-{1-[4-(4-Chloro-benzensulfonyl)-piperazin-1-yl]-ethyl}- |
| | | 4-piperidin-1-yl-quinazoline |
| C3 | VTR-325 | 4-Cyclohexyloxy-2-{1-[4-(4-methoxy-benzensulfonyl)- |
| | | piperazin-1-yl]-ethyl}-quinazoline |
| C4 | Corr-4a | N-[2-(5-Chloro-2-methoxy-phenylamino)-4′-methyl- |
| | | [4,5′]bithiazolyl-2′-yl]-benzamide |
| C5 | Corr-5a | 4,5,7-trimethyl-N-phenylquinolin-2-amine |
| C6 | Corr5c | N-(4-bromophenyl)-4-methylquinolin-2-amine |
| C7 | Genzyme | 2-(4-isopropoxypicolinoyl)-N-(4-pentylphenyl)-1,2,3,4- |
| | cmpd 48 | tetrahydroisoquinoline-3-carboxamide |
| C8 | | N-(2-fluorophenyl)-2-(1H-indol-3-yl)-2-oxoacetamide |
| C9 | KM111060 | 7-chloro-4-(4-(4-chlorophenylsulfonyl)piperazin-1- |
| | | yl)quinoline |
| C11 | Dynasore | (Z)-N′-(3,4-dihydroxybenzylidene)-3-hydroxy-2- |
| | | naphthohydrazide |
| C12 | Corr-2i | N-(4-fluorophenyl)-4-p-tolylthiazol-2-amine |
| C13 | Corr-4c | N-(2-(3-acetylphenylamino)-4′-methyl-4,5′-bithiazol-2′- |
| | | yl)benzamide |
| C14 | Corr-4d | N-(2′-(2-methoxyphenylamino)-4-methyl-5,5′-bithiazol-2- |
| | | yl)benzamide |
| C15 | Corr-2b | N-phenyl-4-(4-vinylphenyl)thiazol-2-amine |
| C16 | Corr-3d | 2-(6-methoxy-4-methylquinazolin-2-ylamino)-5,6- |
| | | dimethylpyrimidin-4(1H)-one |
| C17 | 15jf | N-(2-(5-chloro-2-methoxyphenylamino)-4′-methyl-4,5′- |
| | | bithiazol-2′-yl)pivalamide |
| C18 | CF-106951 | 1-(benzo[d][1,3]dioxol-5-yl)-N-(5-((2-chlorophenyl)(3- |
| | | hydroxypyrrolidin-1-yl)methyl)thiazol-2- |
| | | yl)cyclopropanecarboxamide |
| VX-809 | Lumacaftor | 3-{6-{[1-(2,2-difluoro-1,3-benzodioxol-5- |
| | | yl)cyclopropanecarbonyl]amino}-3-methylpyridin-2- |
| | | yl}benzoic acid |
| Core-cor-II | | |
| RDR1 | | |
| RDR2 | | |
| RDR3 | | |
| Co-Po-22 | | |
| Vx-661 | | |
| Vx-325 | | |
| Vx-422 | | |
| Vx-532 | | |
| glycerol | | |
| TMAO | | |
| (Trimethylamine | | |
| N-oxide) | | |
| taurine | | |
| myo-inositol | | |
| D-sorbitol | | |

10. The method of claim 7, wherein the CFTR potentiator is VX-770 (Kalydeco).

11. The method of claim 3, wherein the therapeutic agent comprises a SYVN1 therapeutic agent that is
(a) an siRNA oligonucleotide having at least 90% identity to SEQ ID NO:18, SEQ ID NO:19, SEQ ID NO:20, SEQ ID NO:21, SEQ ID NO:22, SEQ ID NO:23, SEQ ID NO:24, or SEQ ID NO:25; and/or
(b) a small molecule inhibitor that is LS-101 and/or LS-102.

12. The method of claim 1, wherein the ΔF508-CFTR function has increased membrane stability, the ΔF508-CFTR biosynthesis is increased by proteasome inhibition, wherein ΔF508-CFTR ubiquitination is reduced, ΔF508-CFTR function in primary airway epithelial cultures is partially restored, and/or ΔF508-CFTR mediated transport is improved by at least 10%.

13. The method of claim 1, wherein the cell is a primary airway epithelial cell.

14. The method of claim 1, wherein the therapeutic agent is a DsiRNA.

15. The method of claim 1, further comprising contacting the cell with an auxiliary compound listed in Table 1:
TABLE 1

| Drug (alternative | | |
|---|---|---|
| name) | Developers | Modes of action |
| Bronchitol | Central Sydney Area | Osmotic agent |
| | Health | |
| | Service/Pharmaxis | |
| Ataluren (Translarna) | PTC Therapeutics | Facilitates read-through |
| | | of stop-codons |
| CFTR gene therapy | CFGTC | Gene therapy |
| N-6022 | N30 Pharmaceuticals | GSNOR inhibitor |
| Lynovex (NM-001) | NovaBiotics | Antibacterial, mucolytic |
| OligoG | AlgiPharma | Antibiotic |
| | | oligosaccharide |
| Alpha-1 antitrypsin | Grifols | Anti-inflammatory, |
| | | proteinase inhibitor |
| KB001-A | KaloBios | Anti-inflammatory, |
| | Pharmaceuticals/CFF | monoclonal Fab |
| | | fragment |
| Sildenafil (Revatio) | CFF | Anti-inflammatory, |
| | | phosphodiesterase |
| | | inhibitor |
| Levofloxacin | Aptalis Pharma/CFF | Anti-infective |
| (Aeroquin or MP- | | |
| 376) | | |
| Arikace (inhaled | Insmed/CFF | Anti-infective |
| amikacin) | | |
| AeroVanc (inhaled | Savara | Anti-infective |
| vancomycin) | Pharmaceuticals/CFF | |
| Liprotamase | Eli Lilly | PERT |

16. The method of claim 1, wherein the therapeutic agent is an siRNA oligonucleotide having at least 90% identity to SEQ ID NO:18, SEQ ID NO:19, SEQ ID NO:20, SEQ ID NO:21, SEQ ID NO:22, SEQ ID NO:23, SEQ ID NO:24, or SEQ ID NO:25.

17. The method of claim 1, further comprising a standard cystic fibrosis pharmaceutical, such as an antibiotic.

18. A method of treating a subject having CF comprising administering to the subject an effective amount of a therapeutic agent to alleviate the symptoms of CF, wherein the agent comprises
(a) an anti-NEDD8 RNAi molecule, and/or an anti-NEDD8 antisense oligonucleotide (ASO) or other agent that suppresses NEDD8 expression, or a small molecule drug that interferes with NEDD8 activity or whose actions mimic the biological effects of NEDD8 suppression, and/or
(b) an anti-FBXO2 RNAi molecule, and/or an anti-FBXO2 antisense oligonucleotide (ASO) or other agent that suppresses FBXO2 expression, or a small molecule drug that interferes with FBXO2 activity or whose actions mimic the biological effects of FBXO2 suppression; and/or
(c) an anti-SYVN1 RNAi molecule, and/or an anti-SYVN1 antisense oligonucleotide (ASO) or other agent that suppresses SYVN1 expression, or a small molecule drug that interferes with SYVN1 activity or whose actions mimic the biological effects of SYVN1 suppression.

19. The method of claim 18, further comprising contacting the cell with a CFTR corrector and/or CFTR potentiator.

20. The method of claim 18, wherein the administration is via aerosol, dry powder, bronchoscopic instillation, intra-airway (tracheal or bronchial) aerosol or orally.

21. A pharmaceutical composition for treatment of cystic fibrosis, comprising
(a) an anti-NEDD8 RNAi molecule, and/or an anti-NEDD8 antisense oligonucleotide (ASO) or other agent that suppresses NEDD8 expression, or a small molecule drug that interferes with NEDD8 activity or whose actions mimic the biological effects of NEDD8 suppression, and/or
(b) an anti-FBXO2 RNAi molecule, and/or an anti-FBXO2 antisense oligonucleotide (ASO) or other agent that suppresses FBXO2 expression, or a small molecule drug that interferes with FBXO2 activity or whose actions mimic the biological effects of FBXO2 suppression; and/or
(c) an anti-SYVN1 RNAi molecule, and/or an anti-SYVN1 antisense oligonucleotide (ASO) or other agent that suppresses SYVN1 expression, or a small molecule drug that interferes with SYVN1 activity or whose actions mimic the biological effects of SYVN1 suppression, for use in treating CF.


SEQUENCE LISTING (156 sequences)

SEQ ID NO: 1 - RNA, 99 nt, Homo sapiens
cccuggcauggugugguggggcagcugguguugugaaucaggccguugccaaucagagaacggcuacuucacaacaccagggccacaccacacuacagg

SEQ ID NO: 2 - RNA, 84 nt, Homo sapiens
cguugcugcagcugguguugugaaucaggccgacgagcagcgcauccucuuacccggcuauuucacgacaccaggguugcauca

SEQ ID NO: 3 - RNA, 23 nt, Homo sapiens
agcugguguugugaaucaggccg

SEQ ID NO: 4 - RNA, 18 nt, Artificial Sequence
  Description of Artificial Sequence Synthetic oligonucleotide
ugauucacaacaccagcu

SEQ ID NO: 5 - RNA, 23 nt, Artificial Sequence
  Description of Artificial Sequence Synthetic oligonucleotide
agcugguguugugaaucaggccg

SEQ ID NO: 6 - RNA, 27 nt, Artificial Sequence
  Description of Artificial Sequence Synthetic oligonucleotide
cgucuucacuuuaauuagcaucuucuu

SEQ ID NO: 7 - DNA, 25 bp, Artificial Sequence
  Description of Artificial Sequence Synthetic oligonucleotide
gaagaugcuaauuaaagugaagacg

SEQ ID NO: 8 - RNA, 27 nt, Artificial Sequence
  Description of Artificial Sequence Synthetic oligonucleotide
gucaaucucaaucuccuuuccggucag

SEQ ID NO: 9 - DNA, 25 bp, Artificial Sequence
  Description of Artificial Sequence Synthetic oligonucleotide
gaccggaaaggagauugagauugac

SEQ ID NO: 10 - RNA, 27 nt, Artificial Sequence
  Description of Artificial Sequence Synthetic oligonucleotide
ucccucuuucuccuccacacgcuccuu

SEQ ID NO: 11 - DNA, 25 bp, Artificial Sequence
  Description of Artificial Sequence Synthetic oligonucleotide
ggagcguguggaggagaaagaggga

SEQ ID NO: 12 - RNA, 27 nt, Artificial Sequence
  Description of Artificial Sequence Synthetic oligonucleotide
ggacgcuauggacuaaguuaaggccua

SEQ ID NO: 13 - DNA, 25 bp, Artificial Sequence
  Description of Artificial Sequence Synthetic oligonucleotide
ggccuuaacuuaguccauagcgucc

SEQ ID NO: 14 - RNA, 27 nt, Artificial Sequence
  Description of Artificial Sequence Synthetic oligonucleotide
ucacgcccucacggaucuacauucuag

SEQ ID NO: 15 - DNA, 25 bp, Artificial Sequence
  Description of Artificial Sequence Synthetic oligonucleotide
agaauguagauccgugagggcguga

SEQ ID NO: 16 - RNA, 27 nt, Artificial Sequence
  Description of Artificial Sequence Synthetic oligonucleotide
cacguucucgugcucggacaguagcuu

SEQ ID NO: 17 - DNA, 25 bp, Artificial Sequence
  Description of Artificial Sequence Synthetic oligonucleotide
gcuacuguccgagcacgagaacgtg

SEQ ID NO: 18 - RNA, 27 nt, Artificial Sequence
  Description of Artificial Sequence Synthetic oligonucleotide
gugggccagcgagcaaguucauagcuu

SEQ ID NO: 19 - DNA, 25 bp, Artificial Sequence
  Description of Artificial Sequence Synthetic oligonucleotide
gcuaugaacuugcucgcuggcccac

SEQ ID NO: 20 - RNA, 27 nt, Artificial Sequence
  Description of Artificial Sequence Synthetic oligonucleotide
ucaucugaaacugucuccaacaacucu

SEQ ID NO: 21 - DNA, 25 bp, Artificial Sequence
  Description of Artificial Sequence Synthetic oligonucleotide
aguuguuggagacaguuucagauga

SEQ ID NO: 22 - RNA, 27 nt, Artificial Sequence
  Description of Artificial Sequence Synthetic oligonucleotide
gugggccagcgagcaaguucauagcuu

SEQ ID NO: 23 - DNA, 25 bp, Artificial Sequence
  Description of Artificial Sequence Synthetic oligonucleotide
gcuaugaacuugcucgcuggcccac

SEQ ID NO: 24 - RNA, 27 nt, Artificial Sequence
  Description of Artificial Sequence Synthetic oligonucleotide
gugagguacugguugaucugcagcaug

SEQ ID NO: 25 - DNA, 25 bp, Artificial Sequence
  Description of Artificial Sequence Synthetic oligonucleotide
ugcugcagaucaaccaguaccucac

SEQ ID NO: 26 - RNA, 27 nt, Artificial Sequence
  Description of Artificial Sequence Synthetic oligonucleotide
ccacauguccuuuguauuguacuccug

SEQ ID NO: 27 - DNA, 25 bp, Artificial Sequence
  Description of Artificial Sequence Synthetic oligonucleotide
ggaguacaauacaaaggacaugugg

SEQ ID NO: 28 - DNA, 16 bp, Artificial Sequence
  Description of Artificial Sequence Synthetic oligonucleotide
gcatgcatgcatgcat

SEQ ID NO: 29 - DNA, 14 bp, Artificial Sequence
  Description of Artificial Sequence Synthetic oligonucleotide
gcatgcatgcatgc

SEQ ID NO: 30 - DNA, 14 bp, Artificial Sequence
  Description of Artificial Sequence Synthetic oligonucleotide
gcatttgcagcagc

SEQ ID NO: 31 - RNA, 27 nt, Artificial Sequence
  Description of Artificial Sequence Synthetic oligonucleotide
uacauucuacuuucguugccaugguuu

SEQ ID NO: 32 - RNA, 27 nt, Artificial Sequence
  Description of Artificial Sequence Synthetic oligonucleotide
uuugacauuuccuuaccgaacuuccuu

SEQ ID NO: 33 - RNA, 27 nt, Artificial Sequence
  Description of Artificial Sequence Synthetic oligonucleotide
ugcacaguugguauuuguucuucacag

SEQ ID NO: 34 - RNA, 27 nt, Artificial Sequence
  Description of Artificial Sequence Synthetic oligonucleotide
uucaaccuuaaaucauguuauucccuu

SEQ ID NO: 35 - RNA, 27 nt, Artificial Sequence
  Description of Artificial Sequence Synthetic oligonucleotide
caauuacauucgagacuuaaguucuuu

SEQ ID NO: 36 - RNA, 27 nt, Artificial Sequence
  Description of Artificial Sequence Synthetic oligonucleotide
agaagcuucucacaaacauuagaccag

SEQ ID NO: 37 - RNA, 27 nt, Artificial Sequence
  Description of Artificial Sequence Synthetic oligonucleotide
ugucagaaccauagaagacaccuccuc

SEQ ID NO: 38 - RNA, 27 nt, Artificial Sequence
  Description of Artificial Sequence Synthetic oligonucleotide
uucaguucuucaaaucgggcacgggua

SEQ ID NO: 39 - RNA, 27 nt, Artificial Sequence
  Description of Artificial Sequence Synthetic oligonucleotide
aaucaucaacuauucuucgaucagcac

SEQ ID NO: 40 - RNA, 27 nt, Artificial Sequence
  Description of Artificial Sequence Synthetic oligonucleotide
uaucacaacugcaaguauuuacugcua

SEQ ID NO: 41 - RNA, 27 nt, Artificial Sequence
  Description of Artificial Sequence Synthetic oligonucleotide
guugcacuuguuucuacuucguuagac

SEQ ID NO: 42 - RNA, 27 nt, Artificial Sequence
  Description of Artificial Sequence Synthetic oligonucleotide
acccgauuucaguuuguuaguuugguc

SEQ ID NO: 43 - RNA, 27 nt, Artificial Sequence
  Description of Artificial Sequence Synthetic oligonucleotide
gugggccagcgagcaaguucauagcuu

SEQ ID NO: 44 - RNA, 27 nt, Artificial Sequence
  Description of Artificial Sequence Synthetic oligonucleotide
ucaucugaaacugucuccaacaacucu

SEQ ID NO: 45 - RNA, 27 nt, Artificial Sequence
  Description of Artificial Sequence Synthetic oligonucleotide
ugacaaacagaaauacuaggaaaugca

SEQ ID NO: 46 - RNA, 27 nt, Artificial Sequence
  Description of Artificial Sequence Synthetic oligonucleotide
caguauaaauucaucucugcauguaga

SEQ ID NO: 47 - RNA, 27 nt, Artificial Sequence
  Description of Artificial Sequence Synthetic oligonucleotide
agcacuguucuguucacuccaaucucc

SEQ ID NO: 48 - RNA, 27 nt, Artificial Sequence
  Description of Artificial Sequence Synthetic oligonucleotide
gccacguugauugauuaucugugcuuc

SEQ ID NO: 49 - RNA, 27 nt, Artificial Sequence
  Description of Artificial Sequence Synthetic oligonucleotide
cacaggaucacagacuaggcucugcau

SEQ ID NO: 50 - RNA, 27 nt, Artificial Sequence
  Description of Artificial Sequence Synthetic oligonucleotide
ucuccagacagcucuuaugcacggcac

SEQ ID NO: 51 - RNA, 27 nt, Artificial Sequence
  Description of Artificial Sequence Synthetic oligonucleotide
uuggucuugaccgucagcucguccggg

SEQ ID NO: 52 - RNA, 27 nt, Artificial Sequence
  Description of Artificial Sequence Synthetic oligonucleotide
agcguguauuuccgcgugaagcaccgg

SEQ ID NO: 53 - RNA, 27 nt, Artificial Sequence
  Description of Artificial Sequence Synthetic oligonucleotide
cagugucgaacuguuuguauauggccu

SEQ ID NO: 54 - RNA, 27 nt, Artificial Sequence
  Description of Artificial Sequence Synthetic oligonucleotide
auguuauagagaugcucauucaggugg

SEQ ID NO: 55 - RNA, 27 nt, Artificial Sequence
  Description of Artificial Sequence Synthetic oligonucleotide
uuucaauaucaucuuugcuuaauccac

SEQ ID NO: 56 - RNA, 27 nt, Artificial Sequence
  Description of Artificial Sequence Synthetic oligonucleotide
agguaauugguccuugaauucuuccau

SEQ ID NO: 57 - RNA, 27 nt, Artificial Sequence
  Description of Artificial Sequence Synthetic oligonucleotide
ucauacauuuccucuaucaaauaggcc

SEQ ID NO: 58 - RNA, 27 nt, Artificial Sequence
  Description of Artificial Sequence Synthetic oligonucleotide
auaaucuuugugcuuucucuagaccuu

SEQ ID NO: 59 - RNA, 27 nt, Artificial Sequence
  Description of Artificial Sequence Synthetic oligonucleotide
aaauccauaauguuaguuuaccugcug

SEQ ID NO: 60 - RNA, 27 nt, Artificial Sequence
  Description of Artificial Sequence Synthetic oligonucleotide
aaaugcuuacguugagcuaguuucuua

SEQ ID NO: 61 - RNA, 27 nt, Artificial Sequence
  Description of Artificial Sequence Synthetic oligonucleotide
aaugggcaaguucucaccaaguucuuu

SEQ ID NO: 62 - RNA, 27 nt, Artificial Sequence
  Description of Artificial Sequence Synthetic oligonucleotide
ucccuguaaagcauugguaucuagaau

SEQ ID NO: 63 - RNA, 27 nt, Artificial Sequence
  Description of Artificial Sequence Synthetic oligonucleotide
uuucccuaccucuaaauuccuuucuuu

SEQ ID NO: 64 - RNA, 27 nt, Artificial Sequence
  Description of Artificial Sequence Synthetic oligonucleotide
uauucacuauuucaaagaucaggccua

SEQ ID NO: 65 - RNA, 27 nt, Artificial Sequence
  Description of Artificial Sequence Synthetic oligonucleotide
uacaaacaucaaacaacugucuccuuu

SEQ ID NO: 66 - RNA, 27 nt, Artificial Sequence
  Description of Artificial Sequence Synthetic oligonucleotide
uuagcaaauaucauaugauugcagcau

SEQ ID NO: 67 - RNA, 27 nt, Artificial Sequence
  Description of Artificial Sequence Synthetic oligonucleotide
gguuucuaauuguuucuacugacacuu

SEQ ID NO: 68 - RNA, 27 nt, Artificial Sequence
  Description of Artificial Sequence Synthetic oligonucleotide
uucacagugguaaauuauaggugguuu

SEQ ID NO: 69 - RNA, 27 nt, Artificial Sequence
  Description of Artificial Sequence Synthetic oligonucleotide
cuucauaaacugcucuauuguggcuuu

SEQ ID NO: 70 - RNA, 27 nt, Artificial Sequence
  Description of Artificial Sequence Synthetic oligonucleotide
auuaagcauaaauuaugacuagagcca

SEQ ID NO: 71 - RNA, 27 nt, Artificial Sequence
  Description of Artificial Sequence Synthetic oligonucleotide
augcaauagcuaauuauggucuaccac

SEQ ID NO: 72 - RNA, 27 nt, Artificial Sequence
  Description of Artificial Sequence Synthetic oligonucleotide
cacuuaauauuccuuuaacuguacguc

SEQ ID NO: 73 - RNA, 27 nt, Artificial Sequence
  Description of Artificial Sequence Synthetic oligonucleotide
acucugcauuucuugaagaacucguca

SEQ ID NO: 74 - RNA, 27 nt, Artificial Sequence
  Description of Artificial Sequence Synthetic oligonucleotide
uaaagucagggaagaacauaguucucu

SEQ ID NO: 75 - RNA, 27 nt, Artificial Sequence
  Description of Artificial Sequence Synthetic oligonucleotide
aggaaagacuuuguugucauauggcac

SEQ ID NO: 76 - RNA, 27 nt, Artificial Sequence
  Description of Artificial Sequence Synthetic oligonucleotide
ugucaaauuccacucaccauaaugcua

SEQ ID NO: 77 - RNA, 27 nt, Artificial Sequence
  Description of Artificial Sequence Synthetic oligonucleotide
ugaagugacaauaaccuuaucugcuac

SEQ ID NO: 78 - RNA, 27 nt, Artificial Sequence
  Description of Artificial Sequence Synthetic oligonucleotide
acccgauuucaguuuguuaguuugguc

SEQ ID NO: 79 - RNA, 27 nt, Artificial Sequence
  Description of Artificial Sequence Synthetic oligonucleotide
acuaauauaccaaucaauuaaguccau

SEQ ID NO: 80 - RNA, 27 nt, Artificial Sequence
  Description of Artificial Sequence Synthetic oligonucleotide
uuuaaauagcucuauuuaacuuuggug

SEQ ID NO: 81 - RNA, 27 nt, Artificial Sequence
  Description of Artificial Sequence Synthetic oligonucleotide
gguaguaucugaauucauguaucgcuc

SEQ ID NO: 82 - RNA, 27 nt, Artificial Sequence
  Description of Artificial Sequence Synthetic oligonucleotide
uaggaauucagcuugaaucuacacucc

SEQ ID NO: 83 - RNA, 27 nt, Artificial Sequence
  Description of Artificial Sequence Synthetic oligonucleotide
gugggccagcgagcaaguucauagcuu

SEQ ID NO: 84 - RNA, 27 nt, Artificial Sequence
  Description of Artificial Sequence Synthetic oligonucleotide
gugagguacugguugaucugcagcaug

SEQ ID NO: 85 - RNA, 27 nt, Artificial Sequence
  Description of Artificial Sequence Synthetic oligonucleotide
cgucuucacuuuaauuagcaucuucuu

SEQ ID NO: 86 - RNA, 27 nt, Artificial Sequence
  Description of Artificial Sequence Synthetic oligonucleotide
gucaaucucaaucuccuuuccggucag

SEQ ID NO: 87 - RNA, 27 nt, Artificial Sequence
  Description of Artificial Sequence Synthetic oligonucleotide
ucccucuuucuccuccacacgcuccuu

SEQ ID NO: 88 - RNA, 27 nt, Artificial Sequence
  Description of Artificial Sequence Synthetic oligonucleotide
ggacgcuauggacuaaguuaaggccua

SEQ ID NO: 89 - RNA, 27 nt, Artificial Sequence
  Description of Artificial Sequence Synthetic oligonucleotide
ucacgcccucacggaucuacauucuag

SEQ ID NO: 90 - RNA, 27 nt, Artificial Sequence
  Description of Artificial Sequence Synthetic oligonucleotide
cacguucucgugcucggacaguagcuu

SEQ ID NO: 91 - RNA, 27 nt, Artificial Sequence
  Description of Artificial Sequence Synthetic oligonucleotide
ccacauguccuuuguauuguacuccug

SEQ ID NO: 92 - RNA, 27 nt, Artificial Sequence
  Description of Artificial Sequence Synthetic oligonucleotide
gaaggauuaaauuuaucagcuguccaa

SEQ ID NO: 93 - RNA, 27 nt, Artificial Sequence
  Description of Artificial Sequence Synthetic oligonucleotide
cccucaauacugauucuucucuggcug

SEQ ID NO: 94 - DNA, 25 bp, Artificial Sequence
  Description of Artificial Sequence Synthetic oligonucleotide
accauggcaacgaaaguagaaugta

SEQ ID NO: 95 - DNA, 25 bp, Artificial Sequence
  Description of Artificial Sequence Synthetic oligonucleotide
ggaaguucgguaaggaaaugucaaa

SEQ ID NO: 96 - DNA, 25 bp, Artificial Sequence
  Description of Artificial Sequence Synthetic oligonucleotide
gugaagaacaaauaccaacugugca

SEQ ID NO: 97 - DNA, 25 bp, Artificial Sequence
  Description of Artificial Sequence Synthetic oligonucleotide
gggaauaacaugauuuaagguugaa

SEQ ID NO: 98 - DNA, 25 bp, Artificial Sequence
  Description of Artificial Sequence Synthetic oligonucleotide
agaacuuaagucucgaauguaautg

SEQ ID NO: 99 - DNA, 25 bp, Artificial Sequence
  Description of Artificial Sequence Synthetic oligonucleotide
ggucuaauguuugugagaagcuuct

SEQ ID NO: 100 - DNA, 25 bp, Artificial Sequence
  Description of Artificial Sequence Synthetic oligonucleotide
ggaggugucuucuaugguucugaca

SEQ ID NO: 101 - DNA, 25 bp, Artificial Sequence
  Description of Artificial Sequence Synthetic oligonucleotide
cccgugcccgauuugaagaacugaa

SEQ ID NO: 102 - DNA, 25 bp, Artificial Sequence
  Description of Artificial Sequence Synthetic oligonucleotide
gcugaucgaagaauaguugaugatt

SEQ ID NO: 103 - DNA, 25 bp, Artificial Sequence
  Description of Artificial Sequence Synthetic oligonucleotide
gcaguaaauacuugcaguugugata

SEQ ID NO: 104 - DNA, 25 bp, Artificial Sequence
  Description of Artificial Sequence Synthetic oligonucleotide
cuaacgaaguagaaacaagugcaac

SEQ ID NO: 105 - DNA, 25 bp, Artificial Sequence
  Description of Artificial Sequence Synthetic oligonucleotide
ccaaacuaacaaacugaaaucgggt

SEQ ID NO: 106 - DNA, 25 bp, Artificial Sequence
  Description of Artificial Sequence Synthetic oligonucleotide
gcuaugaacuugcucgcuggcccac

SEQ ID NO: 107 - DNA, 25 bp, Artificial Sequence
  Description of Artificial Sequence Synthetic oligonucleotide
aguuguuggagacaguuucagauga

SEQ ID NO: 108 - DNA, 25 bp, Artificial Sequence
  Description of Artificial Sequence Synthetic oligonucleotide
cauuuccuaguauuucuguuuguca

SEQ ID NO: 109 - DNA, 25 bp, Artificial Sequence
  Description of Artificial Sequence Synthetic oligonucleotide
uacaugcagagaugaauuuauactg

SEQ ID NO: 110 - DNA, 25 bp, Artificial Sequence
  Description of Artificial Sequence Synthetic oligonucleotide
agauuggagugaacagaacagugct

SEQ ID NO: 111 - DNA, 25 bp, Artificial Sequence
  Description of Artificial Sequence Synthetic oligonucleotide
agcacagauaaucaaucaacguggc

SEQ ID NO: 112 - DNA, 25 bp, Artificial Sequence
  Description of Artificial Sequence Synthetic oligonucleotide
gcagagccuagucugugauccugtg

SEQ ID NO: 113 - DNA, 25 bp, Artificial Sequence
  Description of Artificial Sequence Synthetic oligonucleotide
gccgugcauaagagcugucuggaga

SEQ ID NO: 114 - DNA, 25 bp, Artificial Sequence
  Description of Artificial Sequence Synthetic oligonucleotide
cggacgagcugacggucaagaccaa

SEQ ID NO: 115 - DNA, 25 bp, Artificial Sequence
  Description of Artificial Sequence Synthetic oligonucleotide
ggugcuucacgcggaaauacacgct

SEQ ID NO: 116 - DNA, 25 bp, Artificial Sequence
  Description of Artificial Sequence Synthetic oligonucleotide
gccauauacaaacaguucgacactg

SEQ ID NO: 117 - DNA, 25 bp, Artificial Sequence
  Description of Artificial Sequence Synthetic oligonucleotide
accugaaugagcaucucuauaacat

SEQ ID NO: 118 - DNA, 25 bp, Artificial Sequence
  Description of Artificial Sequence Synthetic oligonucleotide
ggauuaagcaaagaugauauugaaa

SEQ ID NO: 119 - DNA, 25 bp, Artificial Sequence
  Description of Artificial Sequence Synthetic oligonucleotide
ggaagaauucaaggaccaauuacct

SEQ ID NO: 120 - DNA, 25 bp, Artificial Sequence
  Description of Artificial Sequence Synthetic oligonucleotide
ccuauuugauagaggaaauguauga

SEQ ID NO: 121 - DNA, 25 bp, Artificial Sequence
  Description of Artificial Sequence Synthetic oligonucleotide
ggucuagagaaagcacaaagauuat

SEQ ID NO: 122 - DNA, 25 bp, Artificial Sequence
  Description of Artificial Sequence Synthetic oligonucleotide
gcagguaaacuaacauuauggautt

SEQ ID NO: 123 - DNA, 25 bp, Artificial Sequence
  Description of Artificial Sequence Synthetic oligonucleotide
agaaacuagcucaacguaagcautt

SEQ ID NO: 124 - DNA, 25 bp, Artificial Sequence
  Description of Artificial Sequence Synthetic oligonucleotide
agaacuuggugagaacuugcccatt

SEQ ID NO: 125 - DNA, 25 bp, Artificial Sequence
  Description of Artificial Sequence Synthetic oligonucleotide
ucuagauaccaaugcuuuacaggga

SEQ ID NO: 126 - DNA, 25 bp, Artificial Sequence
  Description of Artificial Sequence Synthetic oligonucleotide
agaaaggaauuuagagguagggaaa

SEQ ID NO: 127 - DNA, 25 bp, Artificial Sequence
  Description of Artificial Sequence Synthetic oligonucleotide
ggccugaucuuugaaauagugaata

SEQ ID NO: 128 - DNA, 25 bp, Artificial Sequence
  Description of Artificial Sequence Synthetic oligonucleotide
aggagacaguuguuugauguuugta

SEQ ID NO: 129 - DNA, 25 bp, Artificial Sequence
  Description of Artificial Sequence Synthetic oligonucleotide
gcugcaaucauaugauauuugcuaa

SEQ ID NO: 130 - DNA, 25 bp, Artificial Sequence
  Description of Artificial Sequence Synthetic oligonucleotide
gugucaguagaaacaauuagaaacc

SEQ ID NO: 131 - DNA, 25 bp, Artificial Sequence
  Description of Artificial Sequence Synthetic oligonucleotide
accaccuauaauuuaccacugugaa

SEQ ID NO: 132 - DNA, 25 bp, Artificial Sequence
  Description of Artificial Sequence Synthetic oligonucleotide
agccacaauagagcaguuuaugaag

SEQ ID NO: 133 - DNA, 25 bp, Artificial Sequence
  Description of Artificial Sequence Synthetic oligonucleotide
gcucuagucauaauuuaugcuuaat

SEQ ID NO: 134 - DNA, 25 bp, Artificial Sequence
  Description of Artificial Sequence Synthetic oligonucleotide
gguagaccauaauuagcuauugcat

SEQ ID NO: 135 - DNA, 25 bp, Artificial Sequence
  Description of Artificial Sequence Synthetic oligonucleotide
cguacaguuaaaggaauauuaagtg

SEQ ID NO: 136 - DNA, 25 bp, Artificial Sequence
  Description of Artificial Sequence Synthetic oligonucleotide
acgaguucuucaagaaaugcagagt

SEQ ID NO: 137 - DNA, 25 bp, Artificial Sequence
  Description of Artificial Sequence Synthetic oligonucleotide
agaacuauguucuucccugacuuta

SEQ ID NO: 138 - DNA, 25 bp, Artificial Sequence
  Description of Artificial Sequence Synthetic oligonucleotide
gccauaugacaacaaagucuuucct

SEQ ID NO: 139 - DNA, 25 bp, Artificial Sequence
  Description of Artificial Sequence Synthetic oligonucleotide
gcauuauggugaguggaauuugaca

SEQ ID NO: 140 - DNA, 25 bp, Artificial Sequence
  Description of Artificial Sequence Synthetic oligonucleotide
agcagauaagguuauugucacuuca

SEQ ID NO: 141 - DNA, 25 bp, Artificial Sequence
  Description of Artificial Sequence Synthetic oligonucleotide
ccaaacuaacaaacugaaaucgggt

SEQ ID NO: 142 - DNA, 25 bp, Artificial Sequence
  Description of Artificial Sequence Synthetic oligonucleotide
ggacuuaauugauugguauauuagt

SEQ ID NO: 143 - DNA, 25 bp, Artificial Sequence
  Description of Artificial Sequence Synthetic oligonucleotide
ccaaaguuaaauagagcuauuuaaa

SEQ ID NO: 144 - DNA, 25 bp, Artificial Sequence
  Description of Artificial Sequence Synthetic oligonucleotide
gcgauacaugaauucagauacuacc

SEQ ID NO: 145 - DNA, 25 bp, Artificial Sequence
  Description of Artificial Sequence Synthetic oligonucleotide
aguguagauucaagcugaauuccta

SEQ ID NO: 146 - DNA, 25 bp, Artificial Sequence
  Description of Artificial Sequence Synthetic oligonucleotide
gcuaugaacuugcucgcuggcccac

SEQ ID NO: 147 - DNA, 25 bp, Artificial Sequence
  Description of Artificial Sequence Synthetic oligonucleotide
ugcugcagaucaaccaguaccucac

SEQ ID NO: 148 - DNA, 25 bp, Artificial Sequence
  Description of Artificial Sequence Synthetic oligonucleotide
gaagaugcuaauuaaagugaagacg

SEQ ID NO: 149 - DNA, 25 bp, Artificial Sequence
  Description of Artificial Sequence Synthetic oligonucleotide
gaccggaaaggagauugagauugac

SEQ ID NO: 150 - DNA, 25 bp, Artificial Sequence
  Description of Artificial Sequence Synthetic oligonucleotide
ggagcguguggaggagaaagaggga

SEQ ID NO: 151 - DNA, 25 bp, Artificial Sequence
  Description of Artificial Sequence Synthetic oligonucleotide
ggccuuaacuuaguccauagcgucc

SEQ ID NO: 152 - DNA, 25 bp, Artificial Sequence
  Description of Artificial Sequence Synthetic oligonucleotide
agaauguagauccgugagggcguga

SEQ ID NO: 153 - DNA, 25 bp, Artificial Sequence
  Description of Artificial Sequence Synthetic oligonucleotide
gcuacuguccgagcacgagaacgtg

SEQ ID NO: 154 - DNA, 25 bp, Artificial Sequence
  Description of Artificial Sequence Synthetic oligonucleotide
ggaguacaauacaaaggacaugugg

SEQ ID NO: 155 - DNA, 25 bp, Artificial Sequence
  Description of Artificial Sequence Synthetic oligonucleotide
ggacagcugauaaauuuaauccutc

SEQ ID NO: 156 - DNA, 25 bp, Artificial Sequence
  Description of Artificial Sequence Synthetic oligonucleotide
gccagagaagaaucaguauugaggg